(19) United States
(12) Patent Application Publication
(10) Pub. No.: US 2024/0034783 A1
(43) Pub. Date: Feb. 1, 2024

(54) TUMOR-SPECIFIC CLAUDIN 18.2 ANTIBODIES

(71) Applicant: SOTIO BIOTECH A.S., Prague 7 (CZ)

(72) Inventors: Lukas Bammert, Basel (CH); Lenka Kyrych Sadilkova, Horomerice (CZ); Simona Hoskova, Prague 9 (CZ); Iva Valentova, Ceske Budejovice (CZ); Lorenz Waldmeier, Basel (CH); Roger Beerli, Adlikon bei Regensdorf (CH); Ulrich Moebius, Gauting (DE)

(21) Appl. No.: 17/788,226

(22) PCT Filed: Dec. 23, 2020
(86) PCT No.: PCT/EP2020/087735

§ 371 Date: Jun. 22, 2022

(30) Foreign Application Priority Data
Dec. 23, 2019 (EP) 19219359.7
Jan. 17, 2020 (EP) 20152510.2

(51) Int. Cl. C07K 16/28; A61P 35/00

(52) U.S. Cl. CPC C07K 16/28; A61P 35/00; A61K 39/00

(57) ABSTRACT

The invention provides tumor specific anti-CLDN18.2 antibodies or fragments thereof. The antibodies or fragments thereof exhibit increased binding to tumor tissue expressing CLDN 18.2 over healthy tissue expressing CLDN18.2. Further, the antibodies do not exhibit cross-reactivity to CLDN18.1. The invention also provides nucleic acids, vectors, host cells and medical uses.

### BACKGROUND

Tight junctions are multiprotein complexes connecting adjacent epithelial or endothelial cells to form a barrier, preventing molecules from passing in between the cells, and helping to maintain the cell and tissue polarity. Tight junctions consist of three main groups of transmembrane proteins: claudins and occludin, cytoplasmic plaque proteins, and cingulin. They also contain cytoskeletal and signaling proteins, e.g. actin, myosin II, and PKCζ. These proteins interact to maintain the tight junction structure (Yu and Turner 2008).

Claudins form a family of 23 proteins (Hewitt, Agarwal, and Morin 2006). Claudin 18 is a human protein encoded by the CLDN18 gene which forms tight junction strands in epithelial cells. The human CLDN18 can be alternatively spliced with two alternative first exons, resulting in two protein isoforms, CLDN18.1 (or Claudin 18.1) and CLDN18.2 (or Claudin 18.2). CLDN18.2 was first disclosed as Zsig28 protein in WO2000/015659. The two isoforms differ in the N-terminal 69 amino acids encompassing the first extracellular loop. The first extracellular domain spans from amino acid 28 to amino acid 80. Within this stretch there are 8 amino acid differences between CLDN18.1 and CLDN18.2. The two different isoforms are expressed in different tissues, with CLDN18.1 being predominantly expressed in lung tissue whereas CLDN18.2 displays stomach specificity (Niimi et al. 2001). CLDN18.2 expression in normal stomach is restricted to the differentiated short-lived cells of stomach epithelium. CLDN18.2 expression has further been identified in various tumor tissues. For example, CLDN18.2 has been found to be expressed in pancreatic, esophageal, ovarian, and lung tumors, correlating with distinct histologic subtypes (Sahin et al. 2008). The amino acid sequence of human CLDN18.2 protein can be derived from NCBI reference sequence: NP 001002026.1. The sequence is also disclosed as SEQ ID NO: 133.

In view of its restricted expression pattern in normal tissues, and of its ectopic expression in human cancers, CLDN18.2 is an attractive cancer target for antibody therapy of epithelial tumors. A number of studies have been made towards such an antibody therapy. WO2004/047863 identified the splice variants of CLDN18 and screened antibodies against different peptides derived from CLDN18.2: peptide DQWSTQDLYN (SEQ ID NO: 57), N-terminal extracellular of CLDN18.2, independent of glycosylation; peptide NNPVTAVFNYQ (SEQ ID NO: 58), N-terminal extracellular of CLDN18.2, mainly unglycosylated; and peptide STQDLYNNPVTAVF (SEQ ID NO: 59), N-terminal extracellular domain of CLDN18.2, unglycosylated. It also disclosed polyclonal rabbit antibodies screened with a pan-CLDN18 peptide TNFWMSTANMYTG (SEQ ID NO: 60) in the C-terminal extracellular domain common to both CLDN18.1 and CLDN18.2 isoforms. WO2005/113587 discloses antibodies against specific epitopes of CLDN18.2 defined by the peptide sequences: ALMIVGIVLGAIGLLV (SEQ ID NO: 61) and RIGSMEDSAKANMTLTSGIMFIVS (SEQ ID NO: 62). WO2007/059997 discloses CLDN18.2 specific monoclonal antibodies obtained by immunization with the peptide METDTLLLWVLLLWVPGSTGDAAQPARRARRTKLGTELGSTPVWWNSADGRMDQ WSTQDLYNNPVTAVFNYQGLWRSCVRES SGFTECRGYFTLLGLPAMLQAVRAAIQH SGGRSRRARTKTHLRRGSE (SEQ ID NO: 63), including the first extracellular domain of CLDN18.2 with N- and C-terminal extensions. Antibodies obtained by this immunization mediate cell killing by complement dependent cytotoxicity (CDC) and antibody-dependent cell-mediated cytotoxicity (ADCC). Antibody IMAB362, also known as Claudiximab or Zolbetuximab, is disclosed in WO2007/059997 and WO2016/165762. IMAB362 is an IgG1 antibody derived from a murine monoclonal antibody and has been chimerized to display the human IgG1 constant region for clinical use. WO2008/145338 also discloses antibodies binding to overlapping peptides within the first extracellular domain (MDQWSTQDLYNNPVT (SEQ ID NO: 64), LYNNPVTAVFNYQGL (SEQ ID NO: 65), VFNYQGLWRSCVRES (SEQ ID NO: 66), QGLWRSCVRESSGFT (SEQ ID NO: 67), and RSCVRESSGFTECRG (SEQ ID NO: 68)). In an effort to produce antibodies targeting the C-terminal portion of CLDN18.2 for diagnostic purposes to detect CLDN18.2 expression in cells of cancer tissue sections, WO2013/167259 discloses antibodies binding to C-terminal epitopes of CLDN18.2. The sequences of the two epitopes are TEDEVQSYPSKHDYV (SEQ ID NO: 69) and EVQSYPSKHDYV (SEQ ID NO: 70). WO2013/174509 presents combinations of anti-CLDN18.2 antibodies with agents stabilizing γδ T cells or with agents stabilizing or increasing the expression of CLDN18.2. Antibodies may be conjugated to a therapeutic moiety such as a cytotoxin, a drug (e.g. an immunosuppressant) or a radioisotope. WO2014/075788 discloses a method of treatment a cancer disease using a bispecific antibody binding CLDN18.2 and CD3. WO2014/127906 discloses combination agents stabilizing or increasing the expression of CLDN18.2. WO2016/166122 discloses anti-CLDN18.2 monoclonal antibodies that can be highly efficiently internalized upon CLDN18.2 binding and therefore are suitable for antibody-drug conjugate (ADC) development. Furthermore, the conjugation of such antibodies to the drugs DM4 and MMAE using cleavable SPDB or Valine-Citrulline linkers, respectively, is disclosed. However, despite all the antibodies disclosed in the patent applications, only the chimeric IMAB362, disclosed in WO2007/059997 and WO2016/165762, is currently tested in clinical trial. In addition to these antibodies and ADCs, WO2018/006882 discloses chimeric antigen receptors (CAR) based on anti-CLDN18.2 monoclonal antibodies. Antibodies of WO2018/006882 have been humanized and their sequence is disclosed in the Supplementary Materials section associated with Jiang et al 2018 (Jiang et al. 2018). CAR T-cells based on the humanized antibody are currently tested in a phase I clinical trial (ClinicalTrials.gov Identifier: NCT03159819) in patients with advanced gastric adenocarcinoma and pancreatic adenocarcinoma. CN109762067 discloses other anti-CLDN18.2 monoclonal antibodies mediating cell killing by CDC and ADCC. WO2019/173420 discloses anti-CLDN18.2 humanized monoclonal antibodies with ADCC activity. WO2019/175617 discloses anti-CLDN18.2 monoclonal antibodies binding to a different epitope than IMAB362. WO2019/219089 discloses monoclonal antibodies binding to a mutant of CLDN18.2.

CLDN18.2 has been described to exist in different conformations and contains a potential extracellular N-glycosylation site (see WO2007/059997 page 3, first para.), which may lead to potentially different topologies/differential glycosylation between normal and tumor cells (see WO2007/059997 page 4, second para.). However, none of the reported antibodies is preferentially targeting CLDN18.2 expressed on tumor cells. Since CLDN18.2 is expressed not only in tumors, but also in healthy tissue, namely in stomach tissue (Sahin et al. 2008), it clearly would be beneficial to have antibodies targeting only CLDN18.2 expressed in tumor in order to avoid safety issues and side effect very often associated with the on-target effect of therapeutic antibodies to healthy organs/tissues (Hansel et al. 2010), in particular as reported for IMAB362 (Sahin et al. 2018; Tureci et al. 2019).

In addition to binding to targets with high affinity, therapeutic antibodies should maintain their desired properties during development, production, storage and clinical application (in vivo). Antibody stability may be compromised by post-translational modifications (PTM) (Lu et al. 2019; Gervais 2016). Since uncontrolled PTM may lead to antibodies with less than desired efficacy, activity, potency or stability, it is therefore very important while developing therapeutic antibodies to design them with the minimal possible PTMs. PTMs can also have a profound effect on regulatory acceptance, technology transfer or processes and development of biosimilars. The predominant modifications are oxidation, deamidation and isomerization. Further, IMAB362 is a chimeric antibody still having extended mouse sequence, which could lead to antidrug antibodies in some patients, which, e.g. upon repeated application, may lead to decreased efficacy of the treatment.

Therefore, there is a need for improved antibodies specific to CLDN18.2 for use in the treatment of tumor patients.

### Definitions

“Antibodies” or “antibody”, also called “immunoglobulins” (Ig), generally comprise four polypeptide chains, two heavy (H) chains and two light (L) chains, and are therefore multimeric proteins, or comprise an equivalent Ig homologue thereof (e.g., a camelid antibody comprising only a heavy chain, single-domain antibodies (sdAb) or nanobody which can be either be derived from a heavy or light chain). The term “antibodies” includes antibody-based binding protein, modified antibody format retaining target binding capacity. The term “antibodies” also includes full length functional mutants, variants, or derivatives thereof (including, but not limited to, murine, chimeric, humanized and fully human antibodies) which retain the essential epitope binding features of an Ig molecule, and includes dual specific, bispecific, multispecific, and dual variable domain Igs. Ig molecules can be of any class (e.g., IgG, IgE, IgM, IgD, IgA, and IgY), or subclass (e.g., IgG1, IgG2, IgG3, IgG4, IgA1, and IgA2) and allotype. Ig molecules may also be mutated e.g. to enhance or reduce affinity for Fcγ receptors or the neonatal Fc receptor (FcRn).

An “antibody fragment”, as used herein, relates to a molecule comprising at least one polypeptide chain derived from an antibody that is not full length and exhibits target binding. Antibody fragments are capable of binding to the same epitope or target as their corresponding full-length antibody. Antibody fragments include, but are not limited to (i) a Fab fragment, which is a monovalent fragment consisting of the variable light (VL), variable heavy (VH), constant light (CL) and constant heavy 1 (CH1) domains; (ii) a F(ab′) 2 fragment, which is a bivalent fragment comprising two Fab fragments linked by a disulfide bridge at the hinge region (reduction of a F(ab′) 2 fragment result in two Fab′ fragment with a free sulfhydryl group); (iii) a heavy chain portion of a Fab (Fa) fragment, which consists of the VH and CH1 domains; (iv) a variable fragment (Fv) fragment, which consists of the VL and VH domains of a single arm of an antibody; (v) a domain antibody (dAb) fragment, which comprises a single variable domain; (vi) an isolated complementarity determining region (CDR); (vii) a single chain Fv fragment (scFv); (viii) a diabody, which is a bivalent, bispecific antibody in which VH and VL domains are expressed on a single polypeptide chain, but using a linker that is too short to allow for pairing between the two domains on the same chain, thereby forcing the domains to pair with the complementarity domains of another chain and creating two antigen binding sites; (ix) a linear antibody, which comprises a pair of tandem Fv segments (VH-CH1-VH-CH1) which, together with complementarity light chain polypeptides, form a pair of antigen binding regions; (x) Dual-Variable Domain Immunoglobulin; (xi) other non-full length portions of immunoglobulin heavy and/or light chains, or mutants, variants, or derivatives thereof, alone or in any combination.

An “antibody-based binding protein”, as used herein, may represent any protein that contains at least one antibody-derived VH, VL, or CH immunoglobulin domain in the context of other non-immunoglobulin, or non-antibody derived components. Such antibody-based proteins include, but are not limited to (i) Fc-fusion proteins of binding proteins, including receptors or receptor components with all or parts of the immunoglobulin CH domains, (ii) binding proteins, in which VH and or VL domains are coupled to alternative molecular scaffolds, or (iii) molecules, in which immunoglobulin VH, and/or VL, and/or CH domains are combined and/or assembled in a fashion not normally found in naturally occurring antibodies or antibody fragments.

The term “modified antibody format”, as used herein, encompasses antibody-drug-conjugates (ADCs), polyalkylene oxide-modified scFv, monobodies, diabodies, camelid antibodies, domain antibodies, bi- or trispecific antibodies, IgA, or two IgG structures joined by a J chain and a secretory component, shark antibodies, new world primate framework and non-new world primate CDR, IgG4 antibodies with hinge region removed, IgG with two additional binding sites engineered into the CH3 domains, antibodies with altered Fc region to enhance or reduce affinity for Fc gamma receptors, dimerized constructs comprising CH3, VL, and VH, and the like.

The Kabat numbering scheme (Martin and Allemn 2014) has been applied to the disclosed antibodies.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is also to be understood to disclose a group, which preferably consists only of these embodiments.

Where an indefinite or definite article is used when referring to a singular noun, e.g. “a”, “an” or “the”, this includes a plural of that noun unless something else is specifically stated.

Technical terms are used by their common sense. If a specific meaning is conveyed to certain terms, definitions of terms will be given in the following in the context of which the terms are used.

### DESCRIPTION OF THE INVENTION

The inventors have surprisingly identified novel anti-CLDN18.2 antibodies as further described in the following embodiments, which exhibit increased binding to tumor cells expressing CLDN18.2 compared to healthy stomach cells expressing CLDN18.2 and/or have improved stability and/or are humanized while retaining their improved properties.

Therefore, in one embodiment of the invention, the invention provides an antibody or fragment thereof binding to CLDN18.2, wherein the antibody or fragment thereof exhibits increased binding to tumor tissue expressing CLDN18.2 over healthy tissue expressing CLDN18.2. In one embodiment, the healthy cells or tissue used for the comparison are healthy stomach cells or healthy stomach tissue.

Increased binding to tumor tissue by the antibody or fragment thereof provided herein may be shown by bioanalytical methods such as flow cytometry (FC) or immunohistochemistry (IHC), as shown in Examples 4 and 5, respectively. A tumor expressing CLDN18.2 may be generated by subcutaneously injecting CLDN18.2-expressing A549 cells into a Balb/c mouse. The CLDN18.2-expressing A549 cells may be generated as shown in Example 4 and are available under the accession number DSM ACC3360 deposited on 6 Dec. 2019 at the DSMZ-Deutsche Sammlung von Mikroorganismen and Zellkulturen GmbH Inhoffenstr. 7B 38124 Braunschweig DE. The healthy tissue (e.g. healthy stomach tissue) may also originate from the mouse bearing the tumor. Increased binding to tumor tissue over healthy tissue may thus be shown on the tumor tissue and healthy tissue obtained from the same animal.

Increased binding to CLDN18.2 expressed in tumor tissue may be due to posttranslational modification such as differential glycosylation of CLDN18.2, or misfolding of CLDN18.2, when compared to CLDN18.2 expressed in healthy tissue.

Flow cytometry (FC) may be used as a bioanalytical method to test antibody binding. The percentage of CLDN18.2-positive cells can for example be measured by FC for a specific anti-CLDN18.2 antibody. Another possible binding read-out may for example be the ratio of the percentage of CLDN18.2-positive cells in a tumor cell sample versus the percentage of CLDN18.2-positive cells in a cell sample obtained from healthy tissue, such as healthy stomach tissue. Increased binding of an antibody to tumor cells expressing CLDN18.2 generated from CLDN18.2-expressing A549 cells compared to healthy cells, such as healthy stomach cells, may be shown by a ratio of >2, >5, ≥10, preferably ≥15, and more preferably ≥20.

Increased binding of an antibody to tumor cells expressing CLDN18.2 generated from CLDN18.2-expressing A549 cells compared to healthy cells, such as heathy stomach cells, may also be described by showing that the antibody binds at least 2 times more, at least 5 times more, at least 10 times more, preferably at least 15 times more, preferably at least 20 times more tumor cells than healthy cells, such as healthy stomach cells.

Immunohistochemistry (IHC) may be used as a bioanalytical method to test antibody binding. The tissue sample used for IHC should preferably be snap frozen after resection and, once thawed, fixed in acetone as, e.g., shown in Example 5. Since CLDN18.2 is a tight-junction protein in healthy tissue, positive CLDN18.2 staining should result in visualization of a predominantly membranous staining at the cell-cell interface in healthy tissue and/or tumor tissue. Negative CLDN18.2 staining or weak staining should therefore result in absence of membranous staining.

In another embodiment, the invention provides an antibody or fragment thereof binding to CLDN18.2 with a half maximal effective concentration (EC50) value of above 0.4 μg/ml, above μg/ml, preferably above 0.6 μg/ml, but not above 1 μg/ml when measured by flow cytometry (FC) titration on HEK293T cells overexpressing CLDN18.2. HEK293T cells overexpressing CLDN18.2 may be generated as described in Example 3. The EC50 value of the antibody of the invention may be, when measured by flow cytometry (FC) titration on HEK293T cells overexpressing CLDN18.2, between 0.4 and 1 μg/ml, between 0.5 and 1 μg/ml or preferably between 0.6 and 1 μg/ml.

Alternatively, the EC50 value of an antibody of the invention may be compared to the EC50 value of IMAB362 when measured by flow cytometry on HEK293T cells overexpressing CLDN18.2, wherein the EC50 value of the antibody of the invention is at least 1.1 times higher, at least 1.2 times higher, preferably at least 1.5 times higher, more preferably at least 2 times higher, even more preferably at least 2.5 times higher than the EC50 value of IMAB362 but not more than 5 times higher than the EC50 value of IMAB362. The EC50 value of the antibody of the invention may be between 1.1 times higher and 2.5 times higher, between 1.2 times higher and 2.5 times higher, preferably between 1.5 times higher and 2.5 times higher, or more preferably between 2 times higher and 2.5 times higher than the EC50 value of IMAB362 when measured by flow cytometry on HEK293T cells overexpressing CLDN18.2.

In another embodiment, the invention provides an antibody or fragment thereof binding to CLDN18.2 with an EC50 value of above 0.6 μg/ml, above 1 μg/ml, preferably above 1.5 μg/ml, more preferably above 2 μg/ml, but not above 3 μg/ml when measured by flow cytometry titration on PA-TU-8988S-High cells. PA-TU-8988S-High cells may be generated as described in Example 2. The EC50 value of the antibody of the invention, when measured by flow cytometry titration on PA-TU-8988S-High cells, may be between 0.6 and 3 μg/ml, between 1 and 3 μg/ml, preferably between 1.5 and 3 μg/ml, or more preferably between 2 and 3 μg/ml.

Alternatively, the EC50 value of the antibody of the invention may be compared to the EC50 value of IMAB362 when measured by flow cytometry on PA-TU-8988S-High cells, wherein the EC50 value of the antibody of the invention is at least 1.5 times higher, at least 2 times higher, preferably at least 3 times higher, more preferably at least 4 times higher, but not more than 5 times higher than the EC50 value of IMAB362. The EC50 value of the antibody of the invention, when measured by flow cytometry on PA-TU-89885-High cells, may be between 1.5 times higher and 5 times higher, between 2 times higher and 5 times higher, between 3 times higher and 5 times higher or between 4 times higher and 5 times higher than the EC50 value of IMAB362.

In another embodiment, the invention provides an antibody or fragment thereof binding to CLDN18.2 with a maxMFI values within +/−40% of the maxMFI value of IMAB362 when measured by flow cytometry on HEK293T cells overexpressing CLDN18.2. The invention also provides an antibody or fragment thereof binding to CLDN18.2 with maxMFI values equal or up to 2 times higher than the maxMFI value of IMAB362 when measured by flow cytometry on PA-TU-8988S-High cells.

An antibody or functional fragment thereof with increased binding to tumor tissue expressing CLDN18.2 compared to healthy tissue expressing CLDN18.2 may have therapeutic advantages over antibodies unable to discriminate healthy tissue expressing CLDN18.2 from tumor tissue expressing CLDN18.2. Tumor-specific antibodies may not lead to safety issues and side effects, which are very often associated with the on-target effect of therapeutic antibodies in healthy organs/tissues (Hansel et al. 2010). Such undesirable effects have been reported for, e.g., IMAB362 (Sahin et al. 2018; Tureci et al. 2019).

The invention also provides an antibody or fragment thereof binding to CLDN18.2 comprising the heavy chain complementarity determining region (HCDR) HCDR1, HCDR2 and HCDR3 sequences of SEQ ID NO: 21, SEQ ID NO: 22, and SEQ ID NO: 23, respectively and the light chain CDR LCDR1, LCDR2 and LCDR3 sequences of SEQ ID NO: 24, SEQ ID NO: 25, and SEQ ID NO: 26, respectively.

The invention also provides an antibody or fragment thereof binding to CLDN18.2 comprising the heavy chain HCDR3 sequence of SEQ ID NO: 23 and the light chain LCDR3 sequence of SEQ ID NO: 26.

The respective consensus sequences can be found in Table 1. It is understood that any antibody or fragment thereof based on any combination of CDRs derived from the consensus sequences and binding to CLDN18.2 is part of the invention.

TABLE 1
isolated antibody CDR consensus sequences

| CDRs | Sequence | SEQ ID |
|---|---|---|
| HCDR1 | DYAMX | SEQ ID NO: 21 |
| | X in 5^{th} position is H or Y | |
| HCDR2 | WINXYTGKPTYXXXFXG | SEQ ID NO: 22 |
| | X in 4^{th} position is T or A; | |
| | X in 12^{th} position is A or S; | |
| | X in 13^{th} position is D or Q; | |
| | X in 14^{th} position is D or K; | |
| | X in 16^{th} position is K or Q | |
| HCDR3 | AVXYGYTMDA | SEQ ID NO: 23 |
| | X in 3^{rd} position is F or Y | |
| LCDR1 | RXSEDIYSNXA | SEQ ID NO: 24 |
| | X in 2^{nd} position is A or T; | |
| | X in 10^{th} position is L or F | |
| LCDR2 | XXXRLQD | SEQ ID NO: 25 |
| | X in 1^{st} position is S or A; | |
| | X in 2^{nd} position is V or I; | |
| | X in 3^{rd} position is K or N | |
| LCDR3 | LQGSXFPLT | SEQ ID NO: 26 |
| | X in 5^{th} position is K or N | |

Antibody binding or binding affinity is generally expressed in terms of equilibrium association or dissociation constants (Kₐ or K_{d}, respectively), which are in turn reciprocal ratios of dissociation and association rate constants (k_{off} and kₒₙ, respectively). Thus, equivalent affinities may correspond to different rate constants, so long as the ratio of the rate constants remains the same. Binding affinities and/or rate constants can be determined using techniques well known in the art or described herein, such as ELISA, flow cytometry titration, isothermal titration calorimetry (ITC), Biacore (SPR), biolayer inferometry or fluorescent polarization. In some cases, due to the nature of the antigen, the Kₐ or K_{d} of antibodies may be difficult to measure. This is especially true for integral membrane proteins such as Claudins (Hashimoto et al. 2018). In such cases, the integral membrane protein may be expressed as proteoliposomes or lipoparticles. Such lipoparticles may be immobilized on plastic and used in ELISA assay to determine the binding affinity of antibodies to the immobilized antigen. Instead of Kₐ or K_{d} values, half maximal effective concentration (EC50) values may thus be calculated for each tested antibody or functional fragment thereof, reflecting its binding affinity (or strength of binding) to the antigen. Example 2 and FIG. 1 below exemplify ELISA assay binding affinity curves of antibodies with CDRs comprised in the consensus sequences of Table 1. The EC50 value and the maximal binding value can be used for quantification of the binding of the antibodies to CLDN18.2. Example 3 below relates to the calculation of EC50 values by flow cytometry on cells expressing CLDN18.2 of antibodies with CDRs comprised in the consensus sequences of Table 1.

In another embodiment, the invention provides an antibody or fragment thereof binding to CLDN18.2 which comprises the heavy chain CDRs HCDR1, HCDR2 and HCR3 sequences of SEQ ID NO: 21, SEQ ID NO: 126, and SEQ ID NO: 23, respectively and the light chain CDRs LCDR1, LCDR2 and LCDR3 sequences of SEQ ID NO: 24, SEQ ID NO: 25, and SEQ ID NO: 26, respectively.

In one embodiment, the invention relates to an antibody or fragment thereof binding to CLDN18.2, comprising:
a. the HCDR1, HCDR2 and HCDR3 sequences of SEQ ID NO: 1, SEQ ID NO: 15 and SEQ ID NO: 3, respectively, and the LCDR1, LCDR2 and LCDR3 sequences of SEQ ID NO: 4, SEQ ID NO: 5 and SEQ ID NO: 6, respectively;
b. the HCDR1, HCDR2 and HCDR3 sequences of SEQ ID NO: 1, SEQ ID NO: 16 and SEQ ID NO: 3, respectively, and the LCDR1, LCDR2 and LCDR3 sequences of SEQ ID NO: 4, SEQ ID NO: 5 and SEQ ID NO: 6, respectively;
c. the HCDR1, HCDR2 and HCDR3 sequences of SEQ ID NO: 1, SEQ ID NO: 16 and SEQ ID NO: 3, respectively, and the LCDR1, LCDR2 and LCDR3 sequences of SEQ ID NO: 17, SEQ ID NO: 14 and SEQ ID NO: 11, respectively;
d. the HCDR1, HCDR2 and HCDR3 sequences of SEQ ID NO: 1, SEQ ID NO: 16 and SEQ ID NO: 3, respectively, and the LCDR1, LCDR2 and LCDR3 sequences of SEQ ID NO: 18, SEQ ID NO: 19 and SEQ ID NO: 11, respectively;
e. the HCDR1, HCDR2 and HCDR3 sequences of SEQ ID NO: 12, SEQ ID NO: 15 and SEQ ID NO: 3, respectively, and the LCDR1, LCDR2 and LCDR3 sequences of SEQ ID NO: 4, SEQ ID NO: 5 and SEQ ID NO: 6, respectively;
f. the HCDR1, HCDR2 and HCDR3 sequences of SEQ ID NO: 1, SEQ ID NO: 20, and SEQ ID NO: 3, respectively, and the LCDR1, LCDR2 and LCDR3 sequences of SEQ ID NO: 4, SEQ ID NO: 5 and SEQ ID NO: 6, respectively;
g. the HCDR1, HCDR2 and HCDR3 sequences of SEQ ID NO: 1, SEQ ID NO: 20 and SEQ ID NO: 3, respectively, and the LCDR1, LCDR2 and LCDR3 sequences of SEQ ID NO: 18, SEQ ID NO: 19 and SEQ ID NO: 11, respectively;
h. the HCDR1, HCDR2 and HCDR3 sequences of SEQ ID NO: 12, SEQ ID NO: 20 and SEQ ID NO: 8, respectively, and the LCDR1, LCDR2 and LCDR3 sequences of SEQ ID NO: 4, SEQ ID NO: 5 and SEQ ID NO: 6, respectively; or
i. the HCDR1, HCDR2 and HCDR3 sequences of SEQ ID NO: 12, SEQ ID NO: 20 and SEQ ID NO: 8, respectively, and the LCDR1, LCDR2 and LCDR3 sequences of SEQ ID NO: 17, SEQ ID NO: 14 and SEQ ID NO: 11, respectively.

In yet another embodiment, the invention provides an antibody or fragment thereof binding to CLDN18.2, comprising:
a. the HCDR1, HCDR2 and HCDR3 sequences of SEQ ID NO: 1, SEQ ID NO: 2 and SEQ ID NO: 3, respectively, and the LCDR1, LCDR2 and LCDR3 sequences of SEQ ID NO: 4, SEQ ID NO: 5 and SEQ ID NO: 6, respectively;
b. the HCDR1, HCDR2 and HCDR3 sequences of SEQ ID NO: 1, SEQ ID NO: 7 and SEQ ID NO: 8, respectively, and the LCDR1, LCDR2 and LCDR3 sequences of SEQ ID NO: 9, SEQ ID NO: 10 and SEQ ID NO: 11, respectively; or
c. the HCDR1, HCDR2 and HCDR3 sequences of SEQ ID NO: 12, SEQ ID NO: 2 and SEQ ID NO: 3, respectively, and the LCDR1, LCDR2 and LCDR3 sequences of SEQ ID NO: 13, SEQ ID NO: 14 and SEQ ID NO: 11, respectively.

In yet another embodiment, the invention relates to an antibody or fragment thereof binding to CLDN18.2, comprising:
a. a VH sequence of SEQ ID NO: 27 and a VL sequence of SEQ ID NO: 28;
b. a VH sequence of SEQ ID NO: 29 and a VL sequence of SEQ ID NO: 30;
c. a VH sequence of SEQ ID NO: 31 and a VL sequence of SEQ ID NO: 32.

In another embodiment, the invention relates to an antibody or fragment thereof binding to CLDN18.2, comprising:
a. a VH sequence of: SEQ ID NO: 33;
b. a VH sequence of SEQ ID NO: 34;
c. a VH sequence of SEQ ID NO: 35;
d. a VH sequence of SEQ ID NO: 36; or
e. a VH sequence of SEQ ID NO: 37;

and
f. a VL sequence of SEQ ID NO: 38;
g. a VL sequence of SEQ ID NO: 39;
h. a VL sequence of SEQ ID NO: 40; or
i. a VL sequence of SEQ ID NO: 41.

In a further embodiment, the invention relates to an antibody or fragment thereof binding to CLDN18.2, comprising:
a. a VH sequence of SEQ ID NO: 33 and a VL sequence of SEQ ID NO: 38;
b. a VH sequence of SEQ ID NO: 34 and a VL sequence of SEQ ID NO: 38;
c. a VH sequence of SEQ ID NO: 34 and a VL sequence of SEQ ID NO: 39;
d. a VH sequence of SEQ ID NO: 34 and a VL sequence of SEQ ID NO: 40;
e. a VH sequence of SEQ ID NO: 35 and a VL sequence of SEQ ID NO: 38;
f. a VH sequence of SEQ ID NO: 36 and a VL sequence of SEQ ID NO: 41;
g. a VH sequence of SEQ ID NO: 36 and a VL sequence of SEQ ID NO: 40;
h. a VH sequence of SEQ ID NO: 37 and a VL sequence of SEQ ID NO: 41;
i. a VH sequence of SEQ ID NO: 37 and a VL sequence of SEQ ID NO: 38; or
j. a VH sequence of SEQ ID NO: 37 and a VL sequence of SEQ ID NO: 39.

In another embodiment, the invention relates to an antibody binding to CLDN18.2, comprising:
a. the heavy chain sequence of SEQ ID NO: 46 and light chain sequence of SEQ ID NO: 51;
b. the heavy chain sequence of SEQ ID NO: 47 and light chain sequence of SEQ ID NO: 51;
c. the heavy chain sequence of SEQ ID NO: 47 and light chain sequence of SEQ ID NO: 52;
d. the heavy chain sequence of SEQ ID NO: 47 and light chain sequence of SEQ ID NO: 53;
e. the heavy chain sequence of SEQ ID NO: 48 and light chain sequence of SEQ ID NO: 51;
f. the heavy chain sequence of SEQ ID NO: 47 and light chain sequence of SEQ ID NO: 54;
g. the heavy chain sequence of SEQ ID NO: 49 and light chain sequence of SEQ ID NO: 53;
h. the heavy chain sequence of SEQ ID NO: 50 and light chain sequence of SEQ ID NO: 54;
i. the heavy chain sequence of SEQ ID NO: 50 and light chain sequence of SEQ ID NO: 51; or
j. the heavy chain sequence of SEQ ID NO: 50 and light chain sequence of SEQ ID NO: 52.

The constant light chain region CL and the constant heavy chain region CH1 and Fc region of the disclosed antibodies may have the amino acid sequence of SEQ ID NO: 127 and SEQ ID NO: 128, respectively.

In a preferred embodiment, the invention relates to an antibody binding to CLDN18.2, comprising the heavy chain sequence of SEQ ID NO: 46 and light chain sequence of SEQ ID NO: 51.

In a further preferred embodiment, the invention relates to an antibody binding to CLDN18.2, consisting of the heavy chain sequence of SEQ ID NO: 46 and light chain sequence of SEQ ID NO: 51.

The invention also relates to an antibody having an amino acid sequence with at least 80% identity, at least 85%, at least 90%, at least 95% or at least 98% identity to the amino acid sequence of the antibody of the invention, exhibiting increased binding to tumor cells expressing CLDN18.2 compared to healthy stomach cells expressing CLDN18.2.

In one embodiment, the invention relates to an antibody binding to CLDN18.2 and having an amino acid sequence with at least 80% identity, at least 85%, at least 90%, at least 95% or at least 98% identity to an antibody comprising:
a. a VH sequence of SEQ ID NO: 27 and a VL sequence of SEQ ID NO: 28;
b. a VH sequence of SEQ ID NO: 29 and a VL sequence of SEQ ID NO: 30;
c. a VH sequence of SEQ ID NO: 31 and a VL sequence of SEQ ID NO: 32.

In a further embodiment, the invention relates to an antibody binding to CLDN18.2 and having an amino acid sequence with at least 80% identity, at least 85%, at least 90%, at least 95% or at least 98% identity to an antibody comprising:
a. a VH sequence of SEQ ID NO: 33 and a VL sequence of SEQ ID NO: 38;
b. a VH sequence of SEQ ID NO: 34 and a VL sequence of SEQ ID NO: 38;
c. a VH sequence of SEQ ID NO: 34 and a VL sequence of SEQ ID NO: 39;
d. a VH sequence of SEQ ID NO: 34 and a VL sequence of SEQ ID NO: 40;
e. a VH sequence of SEQ ID NO: 35 and a VL sequence of SEQ ID NO: 38;
f. a VH sequence of SEQ ID NO: 36 and a VL sequence of SEQ ID NO: 41;
g. a VH sequence of SEQ ID NO: 36 and a VL sequence of SEQ ID NO: 40;
h. a VH sequence of SEQ ID NO: 37 and a VL sequence of SEQ ID NO: 41;
i. a VH sequence of SEQ ID NO: 37 and a VL sequence of SEQ ID NO: 38; or
j. a VH sequence of SEQ ID NO: 37 and a VL sequence of SEQ ID NO: 39.

In yet a further embodiment, the invention relates to an antibody binding to CLDN18.2 and having an amino acid sequence with at least 80% identity, at least 85%, at least 90%, at least 95% or at least 98% identity to an antibody consisting of the heavy chain sequence of SEQ ID NO: 46 and light chain sequence of SEQ ID NO: 51.

In another embodiment, the Fc domain of the antibody (or antibody fragment when present) may comprise modifications or mutations, such as the modifications or mutations listed in Table 2 below. Such a modification or mutation may be introduced to modulate the effector activity of the Fc domain of the antibody. Modification of antibodies may also include peptide tags added to the C-terminal end of the antibody HC and/or LC chain. Such tags may be used e.g. for protein purification or protein conjugation.

In another embodiment, the invention provides an antibody or fragment thereof binding to CLDN18.2, the antibody being an IgA1, IgA2, IgD, IgE, IgG1, IgG2, IgG3, IgG4, synthetic IgG, IgM, F(ab)₂, Fv, scFv, IgGACH2, F(ab′) 2, scFvCH3, Fab, VL, VH, scFv4, scFv3, scFv2, dsFv, Fv, scFv-Fc, (scFv)₂, a non-depleting IgG, a diabody, a bivalent antibody or Fc-engineered versions thereof. In a preferred embodiment, the antibody is an IgG1 type of antibody. The Fc region of immunoglobulins interacts with multiple Fcγ receptors (FcγR) and complement proteins (e.g. C1q), and mediates immune effector functions, such as elimination of targeted cells via antibody-dependent cellular cytotoxicity (ADCC), antibody-dependent cellular phagocytosis (ADCP) or complement-dependent cytotoxicity (CDC). For therapeutic approaches, it may be beneficial to enhance or silence Fc related effector functions. The type of immunoglobulin (IgA, IgD, IgE, IgG, IgM) may be selected according to the desired effector function of the antibody related to the Fc domain. One may also employ a synthetic immunoglobulin, such as an immunoglobulin with the IgG2 amino acids 118 to 260 and the IgG4 amino acids 261 to 447 or an IgG2 variant with point mutations from IgG4 (e.g. H268Q/V309L/A30S/P331S). Such synthetic immunoglobulins reduce effector functions of the antibody. Fc-engineered immunoglobulins may also be employed to modulate antibody effector function. Table 2 shows example of such Fc engineering. Expression in production cell lines with altered fucosylation may also impact FcγR binding.

TABLE 2
Examples of modifications to modulate antibody effector
function. Unless otherwise noted, the mutations are
on the IgG1 subclass (Wang, Mathieu, and Brezski 2018).

| Engineering and | | |
|---|---|---|
| intended function | Mutation | Reference |
| Enhance ADCC | | |
| Increased FcγRIIIa | F243L/R292P/Y300L/V305I/ | (Stavenhagen |
| binding | P396L | et al. 2007) |
| | S239D/1332E | (Lazar et al. |
| | S298A/E333A/K334A | 2006) |
| | in one heavy chain: | (Shields et al. |
| | L234Y/L235Q/G236W/ | 2001) |
| | S239M/H268D/D270E/ | (Mimoto et al. |
| | S298A, in the opposing | 2013) |
| | heavy chain: D270E/ | |
| | K326D/A330M/K334E | |
| Increased FcγRIIIa | S239D/I332E/A330L | (Lazar et al. |
| binding, decreased | | 2006) |
| FcγRIIb binding | | |
| Enhance ADCP | | |
| Increased FcγRIIa | G236A/S239D/I332E | (Richards et al. |
| binding, Increased | | 2008) |
| FcγRIIIa binding | | |
| Enhance CDC | | |
| Increased C1q | K326W/E333S | (Idusogie et |
| binding | S267E/H268F/S324T | al. 2001) |
| | IgG1/IgG3 cross | (Moore et al. |
| | subclass | 2010) |
| | | (Natsume et |
| | | al. 2008) |
| Hexamerization | E345R/E430G/S440Y | (Diebolder et |
| | | al. 2014) |
| Reduce effector | | |
| function | | |
| Aglycosylated | N297A or N297Q or | (Bolt et al. |
| | N297G | 1993; |
| | | Leabman et |
| | | al. 2013; Tao |
| | | and Morrison |
| | | 1989; Walker |
| | | et al. 1989) |
| Reduced FcγR and | L235E | (Alegre et al. |
| C1q binding | IgG1: L234A/L235A or | 1992) |
| | L234A/L235A/P329G | (Xu et al. |
| | IgG4: F234A/L235A | 2000; Lo et |
| | IgG2/IgG4 cross isotype | al. 2017) |
| | IgG2: H268Q/V309L/ | (Xu et al. |
| | A330S/P331S | 2000) |
| | IgG2: V234A/G237A/ | (Rother et al. |
| | P238S/H268A/V309L/ | 2007) |
| | A330S/P331S | (An et al. |
| | | 2009) |
| | | (Vafa et al. |
| | | 2014) |
| Increase half-life | | |
| Increased FcRn | M252Y/S254T/T256E | (Dall'Acqua |
| Binding at pH 6.0 | | et al. 2002) |
| | M428L/N434S | (Zalevsky et |
| | | al. 2010) |
| Increased coengagement | | |
| Increased FcγRIIb | S267E/L328F | (Chu et al. |
| binding | | 2008) |
| Increased FcγRIIa | N325S/L328F | (Shang et al. |
| binding, decreased | | 2014) |
| FcγRIIIa binding | | |

Half-life of antibodies may also be modulated. The Fc domain plays a central role in the stability and serum half-life of antibodies. For therapeutic approaches, antibody half-life may be reduced by using an antibody fragment missing the Fc domain or with a truncated Fc domain, such as F(ab)2, Fv, scFv, IgGACH2, F(ab′)2, scFvCH3, Fab, VL, VH, scFv4, scFv3, scFv2, dsFv, Fv, scFv-Fc or (scFv)2. The antibodies may also be in the form of diabodies or bivalent antibodies. Diabodies or bivalent antibodies may be used to increase the affinity to the target allowing lower dosage. Functional fragments missing the Fc domain or with truncated Fc domains may also be used in the development of other therapeutic approaches such as chimeric antigen receptor T cell (CART cells) or bispecific T cell engagers (BiTEs). In CAR constructs, one VH and one VL domain are typically connected by a short peptide linker to form a single-chain variable fragment (scFv), and the scFv fragment is further linked to a transmembrane domain and an intracytoplasmic T cell immunoreceptor tyrosine-based activation motif (from e.g. CD3C) and further domains of co-stimulatory molecules (from e.g. CD28, 4-1BB (CD127), or OX40) (Chang and Chen 2017). The VH and VL domains used in the scFv fragment may be the ones of the antibodies listed in Table 3. BiTEs typically consist of the fusion of two scFv of two different antibodies. One scFv domain may be of the isolated antibodies binding CLDN18.2 listed in Table 3, while the other scFv domain is from an antibody that binds e.g. to CD3, CD16, NKG2D, NKp46, CD2, CD28 or CD25. Ample guidance on BiTEs antibody formats and other bispecific antibody formats used for T-cell redirecting may be found in the review by Diego Ellerman (2019).

In another embodiment, the invention provides an antibody or fragment thereof binding to CLDN18.2, the antibody having the constant light chain region (CL) of SEQ ID NO: 127 and preferably the constant heavy chain region CH1 and Fc region of SEQ ID NO: 129 with reduced FcγR binding having the L234A/L235A mutations in the constant heavy chain region CH2. More preferably, the invention provides for an antibody with the constant heavy chain region CH1 and Fc region of SEQ ID NO: 130 having a L234A/L235A/P329G mutation in the constant heavy chain region CH1 and Fc region with even further reduced FcγR binding.

In a another preferred embodiment, the invention relates to an antibody or fragment thereof binding to CLDN18.2, comprising the VH sequence of SEQ ID NO: 33, the VL sequence of SEQ ID NO: 38, the constant light chain region (CL) of SEQ ID NO: 127 and the constant heavy chain region CH1 and Fc region of SEQ ID NO: 129 with L234A/L235A.

In a another preferred embodiment, the invention relates to an antibody or fragment thereof binding to CLDN18.2, consisting of the VH sequence of SEQ ID NO: 33, the VL sequence of SEQ ID NO: 38, the constant light chain region (CL) of SEQ ID NO: 127 and the constant heavy chain region CH1 and Fc region of SEQ ID NO: 129 with L234A/L235A.

In another embodiment, the invention provides an antibody or fragment thereof binding to CLDN18.2, wherein the antibody or fragment thereof is humanized. Humanization of monoclonal antibodies is well-established. The Handbook of Therapeutic Antibodies, Second Edition, gives ample information on humanization of monoclonal antibodies (Saldanha 2014), bioinformatics tools for analysis of such antibodies (Martin and Allemn 2014) and development and manufacture of therapeutic antibodies (Jacobi et al. 2014).

In another embodiment, the antibody or fragment thereof is an isolated antibody or isolated fragment binding to CLDN18.2.

In a further embodiment, the invention provides an antibody or fragment thereof binding to CLDN18.2, wherein the antibody or fragment thereof does not bind to CLDN18.1. Hence, the antibody does not exhibit cross-reactivity or cross-binding to CLDN18.1. Binding of an antibody to a target protein can be tested by flow cytometry on cells expressing the target protein. Specific binding of a tested antibody to its target protein can be visualized on a histogram plot. Such plot results in a peak with high fluorescent signal when the antibody specifically binds to the expressed target protein, and in a peak with low fluorescent signal when the antibody does not, or only very weakly bind to the expressed target protein. The degree of binding can also be expressed in a bar graph showing the maximal mean fluorescent intensity (maxMFI) measured by flow cytometry, with high maxMFI reflecting strong binding and low/no maxMFI reflecting no binding or very weak binding. Comparing maxMFI values for different antibodies in a same experimental set up may also be indicative of the affinity of the antibodies to the target, with a higher maxMFI indicating a lower off rate and higher affinity. Examples of such binding assays can be found in Example 3 and FIGS. 4 and 5.

In another embodiment, the invention provides an antibody or fragment thereof binding to CLDN18.2, the antibody being bound to another moiety. The binding of the antibody or fragment thereof to another moiety may be covalent or no-covalent. The moiety may include radioisotopes, fluorescent tags, histological markers, cytotoxins or cytokines. Covalent binding of the moiety to the antibody may be facilitated by linkers known in the art.

In yet another embodiment, the invention relates to a tumor-specific antibody or fragment thereof that binds to CLDN18.2, wherein the antibody is less susceptible to posttranslational deamidation than IMAB362. In a further embodiment, the invention relates to a tumor-specific antibody or fragment thereof that binds to CLDN18.2, wherein the antibody does not undergo posttranslational deamidation. Posttranslational modifications (PTM) are an important concern in both antibody development and antibody production and storage. —Uncontrolled PTM may lead to antibodies with less efficacy, activity, potency or stability. PTMs may be N-glycosylation, lysine glycation and cysteines capped with other cysteines, glutathione, or other sulfhydryl-containing compounds from cell culture media during bioprocessing, or formation of dimers and higher oligomers due to cysteines linked by covalent disulfide bridges. Among PTMs, deamidation of asparagine (Asn, N) residues, isomerization of aspartate (aspartic acid, Asp, D) residues, and formation of succinimide intermediates are the most frequent modification reactions for therapeutic antibodies during production, storage or in vivo after administration. Deamidation of Asn and isomerization of Asp depend on sequence liabilities, the structural environment and on the storage conditions, particularly the solution pH and storage temperature. These modifications may lead to decreased or even loss of function or biological activity, especially if the affected residues are involved in target binding. Asn and Asp residues are at risk for modifications particularly when they are located in structurally flexible regions such as CDR loops, and when certain other structural prerequisites are met, whereas framework regions have been observed to be comparatively resistant to modifications. In addition to the structural location of Asn and Asp residues, canonic motifs of Asn deamidation and of Asp isomerization have also been identified. These canonical motifs are NG, NS, NN, NT, NH, and DG, DS, DD, DT and DH, respectively (Lu et al. 2019). Upon in-silico analysis, the disclosed antibodies present a DG Asp-isomerization motif in the last amino acid of CDR2 of the VL domain and in the CH2 and CH3 regions of the HC (VL-CDR2 (at position 62), CH2 (at position 282), CH3 (at position 403)).

Isomerization of Asp can be tested by subjecting the antibodies to low pH (i.e. pH 5.5) and heat (i.e. 40° C.) for two weeks, while Asn deamidation of antibodies can be tested by subjecting the antibodies to high pH (i.e. pH 8.0) and heat (i.e. 40° C.) for one week, mimicking production and storage conditions.

The inventors have now shown that the disclosed antibodies, under these harsh conditions, albeit containing Asn and Asp in their CDRs, and bearing an Asp-Gly (DG) Asp-isomerization motif, surprisingly were free of Asn deamidation (see Table 6) and Asp isomerization (see Table 7) and that their binding affinity to CLDN18.2 was not affected. IMAB362 on the other hand showed Asn deamidation under such conditions, inducing a loss of binding affinity (as seen in Table 6 and FIG. 10). The invention thus provides isolated antibodies or fragments thereof that bind to CLDN18.2 and which are less prone than IMAB362 to PTMs during production, storage and clinical application (in vivo) and that warrants for maintained binding affinity to CLDN18.2 during production, storage and clinical application (in vivo).

The invention also provides an antibody binding to the same epitope as an antibody described herein. In one embodiment, the antibody binds to the same epitope as an antibody comprising a heavy chain sequence of SEQ ID NO: 46 and a light chain sequence of SEQ ID NO: 51.

The invention further provides an antibody competing for binding with an antibody described herein. In one embodiment, the antibody competes for binding with an antibody comprising a heavy chain sequence of SEQ ID NO: 46 and a light chain sequence of SEQ ID NO: 51.

The invention further provides an antibody that competitively inhibits binding of an antibody described herein to Claudin 18.2. In one embodiment, the antibody competitively inhibits binding of an antibody comprising a heavy chain sequence of SEQ ID NO: 46 and a light chain sequence of SEQ ID NO: 51 to Claudin 18.2.

Suitable methods to detect binding of antibodies to the same antigen include approaches to map the antigen-antibody interactions. Such approaches have been described in Abbott 2014 (Abbott, Damschroder, and Lowe 2014). Suitable methods to detect competition include competitive assays by epitope binning, as described in Abdiche 2009 (Abdiche et al. 2009). Suitable method for detecting competitive inhibition include ELISA assays.

According to one embodiment, the invention provides nucleic acid sequences encoding the isolated tumor-specific antibodies or functional fragments thereof that bind CLDN18.2. The nucleic acid sequences may encode for the CDRs alone, for the VH and VL regions, or for the entire heavy and light chains of the antibodies. These nucleic acid sequences may be found in Table 3. The nucleic acid sequence may also encode for F(ab)₂, Fv, scFv, IgGACH2, F(ab′)₂, scFvCH3, Fab, VL, VH, scFv4, scFv3, scFv2, dsFv, Fv, scFv-Fc, (scFv)₂, a non-depleting IgG, a diabody, a bivalent antibody or Fc-engineered versions thereof. The encoded immunoglobin may be an IgA1, IgA2, IgD, IgE, IgG1, IdG2, IgG3, IgG4, synthetic IgG, IgM or mutated and Fc-engineered versions thereof.

In yet another embodiment, the nucleic acid sequence may also encode a CAR construct that binds to CLDN18.2. Ample guidance on construction of CART cells may be found in Chang and Chen (2017) or June and Sadelain (2018). In one embodiment, the invention provides a T cell that has been genetically engineered to produce an artificial T-cell receptor, e.g. a chimeric antigen receptor (CAR), wherein the artificial T-cell receptor comprises the antibody or functional fragment thereof of the present invention that binds to CLDN18.2.

In yet another embodiment, the invention provides a tumor-specific antibody-based binding protein that specifically binds to CLDN18.2. Such binding protein may contain at least a CLDN18.2 binding domain of the disclosed antibodies and another protein domain not related to antibodies. The invention also provides a modified antibody format that binds to CLDN18.2.

The invention also provides an expression vector comprising a nucleic acid of the invention or a degenerate nucleic acid as a result of codon degeneracy. The expression vector may be an expression vector for protein expression in mammalian cells, bacteria, fungal or insect cells, and chosen for the type of host cell bearing the expression vector comprising the nucleic acid encoding the antibodies or functional fragments thereof. Ample guidance for the construction of such vectors may be found in Green and Sambrook (Green and Sambrook 2012).

In another embodiment, the invention provides a host cell comprising a nucleic acid or an expression vector of the present invention. The host cell may be a mammalian cell or cell line, a bacterial cell, a fungal cell or an insect cell.

In another embodiment, the invention relates to an antibody or fragment thereof binding to CLDN18.2, the nucleic acid encoding the antibody or fragment thereof, the vector comprising the nucleic acid or the host cells comprising the nucleic acid or the vector, for use in the treatment of a subject that is suffering from a neoplastic disease.

In another embodiment, the invention relates to an antibody or fragment thereof binding to CLDN18.2, the nucleic acid encoding the antibody or fragment thereof, the vector comprising the nucleic acid or the host cells comprising the nucleic acid or the vector, for use in the treatment of a subject that is at risk of developing a neoplastic disease, and/or for use in the treatment of a subject being diagnosed for a neoplastic disease.

The disclosed antibodies or fragments thereof may be used as monotherapy. In a preferred embodiment, the disclosed antibodies or fragments thereof are used in combination with the established standard of care of the neoplastic disease.

The neoplastic disease may be at least one disease selected from the group consisting of pancreatic, gastric, esophageal, ovarian and lung cancer. It is understood that the neoplastic disease to be treated expresses CLDN18.2.

In one embodiment, the subject is a mammal. In a preferred embodiment, the subject is a human.

Another embodiment of the invention provides a method for treating a neoplastic disease, including pancreatic, gastric, esophageal, ovarian or lung cancer, with an antibody or functional fragment thereof that binds to CLDN18.2, wherein the method comprises administering a pharmaceutically effective amount of the antibody or functional fragment thereof to a subject in need thereof. The method of treatment may be a monotherapy or preferably a combination therapy with the established standard of care of the neoplastic disease.

The amino acid sequence of human CLDN18.2 protein can be derived from NCBI reference sequence: NP 001002026.1. The sequence is also disclosed as SEQ ID NO: 133.

### DESCRIPTION OF DRAWINGS

FIG. 1: Evaluation by ELISA of the binding to lipoparticles containing CLDN18.2 or null-lipoparticles of selected chimeric and humanized anti-CLDN18.2 antibodies as indicated. A. Chimeric antibodies cCl1-1, cCl1-2, cCl1-3, IMAB362 and only secondary antibody; B. Humanized antibodies hCl1a to hCl1j, chimeric cCl1-1, IMAB362 and only secondary antibody. All newly generated antibodies bind to liposomal CLDN18.2.

FIG. 2: Sorting of PA-TU-8988S cells for expression levels of CLDN18.2. A. FC profile of PA-TU-9888S stained with IMAB362. B. FC profile of PA-TU-8988S cells sorted by FACS for high expression of CLDN18.2.

FIG. 3: Generation of HEK293T cells overexpressing huCLDN18.2. HEK293T cells, not expressing endogenously CLDN18.2, were transfected with a plasmid coding for huCLDN18.2 to stably express CLDN18.2 or coding for huCLDN18.1 to stably express CLDN18.1. The expression was analyzed by FC after staining with IMAB362, and a panCLDN18.1 antibody or an anti-human IgG secondary antibody only. A. FC profile of un-transfected HEK293T cells. B. FC profile of transfected HEK293T cells stably expressing CLDN18.1. C. FC profile of transfected HEK293T cells stably expressing CLDN18.2.

FIG. 4: Flow cytometry binding assay of chimeric cCl1-1, cCl1-2 and cCl1-3 antibodies to pre-B cell L11 cells overexpressing CLDN18.1 or CLDN18.2. The chimeric antibodies bind to CLDN18.2 and not to CLDN18.1. IMAB362 was used as positive binding control.

FIG. 5: Flow cytometry binding assay of humanized hCl1a to hCl1j antibodies to HEK293T cells overexpressing CLDN18.1 or CLDN18.2. The humanized antibodies bind to CLDN18.2 and not to CLDN18.1. IMAB362 and cCL1-1 were used as positive binding control.

FIG. 6: FACS expression profiles of A549 cells overexpressing CLDN18.2. A549 cells, not expressing endogenously CLDN18.2, were stably transfected with a plasmid coding for CLDN18.2 and the expression of CLDN18.2 was analyzed by FACS using IMAB362.

FIG. 7: Flow cytometry live-cell staining. Graph representing the percentage of isolated single cells bound by CLDN18.2 antibodies (cCl1-1, hCl1a, hCl1b, hCl1c, hCl1f and IMAB362). Single cells were isolated either from a mouse tumor expressing CLDN18.2 arising from injected A549 cells overexpressing CLDN18.2 (solid bars) or from a mouse healthy stomach expressing CLDN18.2 (open bars).

FIG. 8: Staining of frozen stomach tissue. Frozen tissue slides of mouse healthy stomach tissue expressing CLDN18.2 have been stained with hCl1a (A), hCl1b (B), hCl1c (C), hCl1f (D) or IMAB362 (E) antibodies. Pictures are representative IHC images.

FIG. 9: Staining of frozen tumor tissue arising from injected A549 cells overexpressing CLDN18.2. Frozen tissue slides of mouse tumor expressing CLDN18.2 have been stained with hCl1a (A), hCl1f (B), IMAB362 (C) or the Abcam 34H14L15 pan-CLDN18 antibodies. Pictures are representative IHC images.

FIG. 10: Effect of deamidation on the binding activity of IMAB362. The affinity of IMAB362 to CLDN18.2 decreases after deamidation.

### EXAMPLES

### Example 1: Generation of Chimeric and Humanized Antibodies

Techniques to generate monoclonal antibodies have been well-established. The Handbook of Therapeutic Antibodies, Second Edition (2014), gives ample information on these techniques, such as the production of monoclonal antibodies by immunization of mice or rats (Moldenhauer 2014), humanization of monoclonal antibodies (Saldanha 2014), bioinformatics tools for analysis of antibodies (Martin and Allemn 2014) or development and manufacture of therapeutic antibodies (Jacobi et al. 2014). In brief, monoclonal antibodies against CLDN18.2 were generated by DNA immunization of rats with a plasmid coding for the human CLDN18.2 cDNA (huCLDN18.2) (NCBI Reference Sequence: NM 001002026.3). The specific reactivity of rat immune sera against huCLDN18.2 was analyzed by flow cytometry (FC analysis) and ELISA. Hybridoma clones were subsequently generated from lymphocytes isolated from the immunized rats to obtain chimeric antibodies. Three clones were identified as being CLDN18.2-specific, resulting in the chimeric antibodies named cCl1-1, cCl1-2 and cCl1-3 with similar CDRs (see Table 3). Subsequently, cCl1-1 cCl1-2 and cCl1-3 were humanized, resulting in 10 humanized clones named hCl1a, hCl1b, hCl1c, hCl1d, hCl1e, hCl1f, hCl1g, hCl1h, hCl1i and hCl1j antibodies (see Table 3).

As a control, the IMAB362 antibody was synthesized using the sequences of the heavy (SEQ ID NO: 55) and light chain (SEQ ID NO: 56) as published in WO2013/174509 and designated as monoclonal antibody 182-D1106-362, accession no. DSM ACC2810, deposited on 26 October, 2006 at the DSMZ-Deutsche Sammlung von Mikroorganismen and Zellkulturen GmbH Inhoffenstr. 7 B 38124 Braunschweig DE.

TABLE 3
antibody nucleic acid and amino acid sequences

| | | |
|---|---|---|
| NAME | SEQUENCE | SEQ ID NO |

cCl1-1

| | | |
|---|---|---|
| HCDR1 | DYAMH | SEQ ID NO: 1 |
| HCDR2 | WINTYTGKPTYADDEKG | SEQ ID NO: 2 |
| HCDR3 | AVFYGYTMDA | SEQ ID NO: 3 |
| VH | QIQLVQSGPELKKPGESVKISCKASGYTFTDYAMHWVKQAPGK | SEQ ID NO: 27 |
| | GLKWMGWINTYTGKPTYADDFKGREVESLEASASTANLQISNL | |
| | KNEDTATYFCARAVFYGYTMDAWGQGTSVTVSS | |
| HCDR1 | gactacgcgatgcac | SEQ ID NO: 71 |
| HCDR2 | tggatcaacacgtacacggggaagccgacatacgcggacgact | SEQ ID NO: 72 |
| | tcaagggg | |
| HCDR3 | gccgtcttctacggatatacgatggacgcg | SEQ ID NO: 73 |
| VH | cagatccagctcgtccagagcgggccggagctgaagaagccgg | SEQ ID NO: 74 |
| | gggagagcgtgaagatctcgtgcaaggcgagcggatatacgtt | |
| | cacggactacgcgatgcactgggtcaagcaagcgccggggaaa | |
| | gggctgaagtggatggggtggatcaacacgtacacggggaagc | |
| | cgacatacgcggacgacttcaaggggcgattcgtgttctcgct | |
| | ggaggcgagcgcgagcacggcgaacctgcaaatctcgaacctg | |
| | aagaacgaggacacggcgacgtacttctgcgcgcgggccgtct | |
| | tctacggatatacgatggacgcgtgggggcagggtaccagcgt | |
| | gacggtctcgagc | |
| LCDR1 | RASEDIYSNLA | SEQ ID NO: 4 |
| LCDR2 | SVKRLQD | SEQ ID NO: 5 |
| LCDR3 | LQGSNFPLT | SEQ ID NO: 6 |
| VL | DIQMTQSPASLSASLGETISIACRASEDIYSNLAWYQQKSGKS | SEQ ID NO: 28 |
| | PQLLIFSVKRLQDGVPSRFSGSGSGTQYSLKISGMQPEDEGDY | |
| | FCLQGSNFPLTFGSGTKLEIK | |
| LCDR1 | cgggcgagcgaggacatctactcgaacctggcg | SEQ ID NO: 75 |
| LCDR2 | tccgtcaagcggctgcaagac | SEQ ID NO: 76 |
| LCDR3 | ctgcaagggagcaacttcccgctgacg | SEQ ID NO: 77 |
| VL | gacatccagatgacgcagagcccggcgtcgctgagcgcgagcc | SEQ ID NO: 78 |
| | tgggggagacgatctcgatcgcgtgccgggcgagcgaggacat | |
| | ctactcgaacctggcgtggtatcaacagaagagcgggaagagc | |
| | ccgcagctgctgatcttctccgtcaagcggctgcaagacggcg | |
| | tcccgagccgattctcggggagcgggagcgggacgcagtactc | |
| | gctgaagatctcggggatgcagccggaggacgagggggactac | |
| | ttctgcctgcaagggagcaacttcccgctgacgttcgggtcgg | |
| | gtaccaaactcgagatcaaa | |

cCl1-2

| | | |
|---|---|---|
| HCDR1 | DYAMH | SEQ ID NO: 1 |
| HCDR2 | WINAYTGKPTYADDEKG | SEQ ID NO: 7 |
| HCDR3 | AVYYGYTMDA | SEQ ID NO: 8 |
| VH | QIQLVQSGPELKKPGESVKISCKTSGYTFTDYAMHWVKQGPGK | SEQ ID NO: 29 |
| | GMKWMGWINAYTGKPTYADDFKGRFVLSLEASASTANLQISNL | |
| | KNEDTATYFCARAVYYGYTMDAWGQGTSVIVSS | |
| HCDR1 | gactacgcgatgcac | SEQ ID NO: 71 |
| HCDR2 | tggatcaacgcgtacacggggaagccgacctacgcggacgact | SEQ ID NO: 79 |
| | tcaagggg | |
| HCDR3 | gccgtctactacggatatacgatggac | SEQ ID NO: 80 |
| VH | cagatccagctcgtccagagcgggccggagctgaagaagccgg | SEQ ID NO: 81 |
| | gggagagcgtgaagatctcgtgcaagacgagcggatatacgtt | |
| | cacggactacgcgatgcactgggtcaagcaggggccagggaaa | |
| | gggatgaagtggatggggtggatcaacgcgtacacggggaagc | |
| | cgacctacgcggacgacttcaaggggcgattcgtgctgagcct | |
| | ggaggcgagcgcctcgacggcgaacctgcaaatctcgaacctg | |
| | aagaacgaggacacggcgacgtacttctgcgcgcgggccgtct | |
| | actacggatatacgatggacgcgtgggggcagggtaccagcgt | |
| | gatcgtctcgagc | |
| LCDR1 | RTSEDIYSNFA | SEQ ID NO: 9 |
| LCDR2 | SVNRLQD | SEQ ID NO: 10 |
| LCDR3 | LQGSKFPLT | SEQ ID NO: 11 |
| VL | DIQMTQSPASLSASLGETISIECRTSEDIYSNFAWFQQKSGKS | SEQ ID NO: 30 |
| | PQLLIYSVNRLQDGVPSRFSGSGSGTQYSLKISGMQPEDEGDY | |
| | FCLQGSKFPLTFGSGTKLEIK | |
| LCDR1 | cggacgagcgaggacatctactcgaacttcgcg | SEQ ID NO: 82 |
| LCDR2 | tcagtcaaccggctgcaagac | SEQ ID NO: 83 |
| LCDR3 | ctgcaagggagcaagttcccgctgacg | SEQ ID NO: 84 |
| VL | gacatccagatgacgcagagcccggcgagcctgagcgcgagcc | SEQ ID NO: 85 |
| | tgggggagacgatctcgatcgagtgccggacgagcgaggacat | |
| | ctactcgaacttcgcgtggttccagcagaagagcgggaagagc | |
| | ccgcagctgctgatctactcagtcaaccggctgcaagacggcg | |
| | tcccgagccgattctcggggagcgggagcgggacgcagtactc | |
| | gctgaagatctcggggatgcagccggaggacgagggggactac | |
| | ttctgcctgcaagggagcaagttcccgctgacgttcgggagcg | |
| | gtaccaaactcgagatcaaa | |

cCl1-3

| | | |
|---|---|---|
| HCDR1 | DYAMY | SEQ ID NO: 12 |
| HCDR2 | WINTYTGKPTYADDEKG | SEQ ID NO: 2 |
| HCDR3 | AVFYGYTMDA | SEQ ID NO: 3 |
| VH | QIQLVQSGPELKKPGESVKISCKASGYTFTDYAMYWVKQVPGK | SEQ ID NO: 31 |
| | GLRWMGWINTYTGKPTYADDFKGREVESLEASASTANLQISNL | |
| | KNEDTATY FCARAVFYGYTMDAWGQGTSVTVSS | |
| HCDR1 | gactacgcgatgtac | SEQ ID NO: 86 |
| HCDR2 | tggatcaacacgtacacggggaagccgacctacgcggacgact | SEQ ID NO: 87 |
| | tcaagggg | |
| HCDR3 | gccgtcttctacggatatacgatggacgcg | SEQ ID NO: 73 |
| VH | cagatccagctcgtccagagcgggccggagctgaagaagccgg | SEQ ID NO: 88 |
| | gggagagcgtgaagatctcgtgcaaggcgagcggatatacgtt | |
| | cacggactacgcgatgtactgggtcaagcaagtgccggggaaa | |
| | gggctgcgatggatggggtggatcaacacgtacacggggaagc | |
| | cgacctacgcggacgacttcaaggggcgattcgtgttctcgct | |
| | ggaggcgagcgcgagcacggcgaacctgcaaatctcgaacctg | |
| | aagaacgaggacacggcgacgtacttctgcgcgcgggccgtct | |
| | tctacggatatacgatggacgcgtgggggcagggtaccagcgt | |
| | gacggtctcgagc | |
| LCDR1 | RTSEDIYSNLA | SEQ ID NO: 13 |
| LCDR2 | AIKRLQD | SEQ ID NO: 14 |
| LCDR3 | LQGSKFPLT | SEQ ID NO: 11 |
| VL | DIQMTQSPASLSASLGETISIACRTSEDIYSNLAWYQQKSGKS | SEQ ID NO: 32 |
| | POLLIFAIKRLQDGVPSRFSGSGSGTQYSLKISGMQPEDEGDY | |
| | FCLQGSKFPLTFGSGTKLEIK | |
| LCDR1 | cggacgagcgaggacatctactcgaacctggcg | SEQ ID NO: 89 |
| LCDR2 | gcgatcaagcggctgcaagac | SEQ ID NO: 90 |
| LCDR3 | ctgcaagggagcaagttcccgctgacg | SEQ ID NO: 84 |
| VL | gacatccagatgacgcagagcccggcgagcctgagcgcgagcc | SEQ ID NO: 91 |
| | tgggggagacgatctcgatcgcgtgccggacgagcgaggacat | |
| | ctactcgaacctggcgtggtatcaacagaagagcgggaagagc | |
| | ccgcagctgctgatcttcgcgatcaagcggctgcaagacggcg | |
| | tcccgagccgattctcggggagcgggagcgggacgcagtactc | |
| | gctgaagatctcggggatgcagccggaggacgagggggactac | |
| | ttctgcctgcaagggagcaagttcccgctgacgttcgggtcgg | |
| | gtaccaaactcgagatcaaa | |

hCl1a

| | | |
|---|---|---|
| HCDR1 | DYAMH | SEQ ID NO: 1 |
| HCDR2 | WINTYTGKPTYAQKFQG | SEQ ID NO: 15 |
| HCDR3 | AVFYGYTMDA | SEQ ID NO: 3 |
| VH | QVQLVQSGAEVKKPGASVKVSCKASGYTFTDYAMHWVRQAPGQ | SEQ ID NO: 33 |
| | RLEWMGWINTYTGKPTYAQKFQGRVTITRDTSASTAYMELSSL | |
| | RSEDTAVYYCARAVFYGYTMDAWGQGTLVTVSS | |
| Heavy chain | QVQLVQSGAEVKKPGASVKVSCKASGYTFTDYAMHWVRQAPGQ | SEQ ID NO: 46 |
| | RLEWMGWINTYTGKPTYAQKFQGRVTITRDTSASTAYMELSSL | |
| | RSEDTAVYYCARAVFYGYTMDAWGQGTLVTVSSASTKGPSVEP | |
| | LAPSSKSTSGGTAALGCLVKDYFPEPVTVSWNSGALTSGVHTF | |
| | PAVLQSSGLYSLSSVVTVPSSSLGTQTYICNVNHKPSNTKVDK | |
| | KVEPKSCDKTHTCPPCPAPELLGGPSVFLFPPKPKDTLMISRT | |
| | PEVTCVVVDVSHEDPEVKENWYVDGVEVHNAKTKPREEQYNST | |
| | YRVVSVLTVLHQDWLNGKEYKCKVSNKALPAPIEKTISKAKGQ | |
| | PREPQVYTLPPSRDELTKNQVSLTCLVKGFYPSDIAVEWESNG | |
| | QPENNYKTTPPVLDSDGSFFLYSKLTVDKSRWQQGNVFSCSVM | |
| | HEALHNHYTQKSLSLSPGK | |
| HCDR1 | gactacgcgatgcac | SEQ ID NO: 71 |
| HCDR2 | tggatcaatacatacacggggaagccgacttatgcgcaaaaat | SEQ ID NO: 92 |
| | tccaagga | |
| HCDR3 | gcggtcttctacggatatacgatggatgcc | SEQ ID NO: 93 |
| VH | caggtccaactagtccaaagcggggcggaagtcaagaagcccg | SEQ ID NO: 94 |
| | gagcatccgtcaaagtcagctgcaaggcgagcggatatacatt | |
| | tacggactacgcgatgcactgggtcaggcaagcccctgggcaa | |
| | aggctcgaatggatgggatggatcaatacatacacggggaagc | |
| | cgacttatgcgcaaaaattccaaggaagagtcacaattacgcg | |
| | ggatacatccgcatctaccgcctacatggagctaagctcgctg | |
| | cggagcgaggatacggcggtctactattgcgcccgagcggtct | |
| | tctacggatatacgatggatgcctgggggcagggtaccctggt | |
| | cacggtctcgagc | |
| LCDR1 | RASEDIYSNLA | SEQ ID NO: 4 |
| LCDR2 | SVKRLQD | SEQ ID NO: 5 |
| LCDR3 | LQGSNFPLT | SEQ ID NO: 6 |
| VL | DIQMTQSPSSLSASVGDRVTITCRASEDIYSNLAWYQQKPGKA | SEQ ID NO: 38 |
| | PKLLIFSVKRLQDGVPSRFSGSGSGTDFTLTISSLOPEDFATY | |
| | YCLQGSNFPLTFGQGTKVEIK | |
| Light chain | DIQMTQSPSSLSASVGDRVTITCRASEDIYSNLAWYQQKPGKA | SEQ ID NO: 51 |
| | PKLLIFSVKRLQDGVPSRFSGSGSGTDFTLTISSLQPEDFATY | |
| | YCLQGSNFPLTFGQGTKVEIKRTVAAPSVFIFPPSDEQLKSGT | |
| | ASVVCLLNNFYPREAKVQWKVDNALQSGNSQESVTEQDSKDST | |
| | YSLSSTLTLSKADYEKHKVYACEVTHQGLSSPVTKSENRGEC | |
| LCDR1 | agggcctccgaagacatctactccaacctggca | SEQ ID NO: 95 |
| LCDR2 | agcgtcaaaagactacaagat | SEQ ID NO: 96 |
| LCDR3 | ttgcaaggaagcaatttccccttgact | SEQ ID NO: 97 |
| VL | gacattcaaatgacgcaaagcccatcatcgctgagcgcatcgg | SEQ ID NO: 98 |
| | tcggggatagagtcaccataacatgcagggcctccgaagacat | |
| | ctactccaacctggcatggtatcaacaaaaaccggggaaggct | |
| | ccgaagctgctgatatttagcgtcaaaagactacaagatggag | |
| | taccgagccgattttcgggaagcgggagcgggacggatttcac | |
| | gctgaccatatcaagtttgcaaccggaggattttgcgacatac | |
| | tattgcttgcaaggaagcaatttccccttgactttcgggcaag | |
| | gtaccaaggtcgagatcaaa | |

hCl1b

| | | |
|---|---|---|
| HCDR1 | DYAMH | SEQ ID NO: 1 |
| HCDR2 | WINTYTGKPTYSQKFQG | SEQ ID NO: 16 |
| HCDR3 | AVFYGYTMDA | SEQ ID NO: 3 |
| VH | QVQLVQSGAEVKKPGASVKVSCKASGYTFTDYAMHWVRQAPGQ | SEQ ID NO: 34 |
| | RLEWMGWINTYTGKPTYSQKFQGRVTITRDTSASTAYMELSSL | |
| | RSEDTAVYYCARAVFYGYTMDAWGQGTLVTVSS | |
| Heavy chain | QVQLVQSGAEVKKPGASVKVSCKASGYTFTDYAMHWVRQAPGQ | SEQ ID NO: 47 |
| | RLEWMGWINTYTGKPTYSQKFQGRVTITRDTSASTAYMELSSL | |
| | RSEDTAVYYCARAVFYGYTMDAWGQGTLVTVSSASTKGPSVFP | |
| | LAPSSKSTSGGTAALGCLVKDYFPEPVTVSWNSGALTSGVHTF | |
| | PAVLQSSGLYSLSSVVTVPSSSLGTQTYICNVNHKPSNTKVDK | |
| | KVEPKSCDKTHTCPPCPAPELLGGPSVELFPPKPKDTLMISRT | |
| | PEVTCVVVDVSHEDPEVKENWYVDGVEVHNAKTKPREEQYNST | |
| | YRVVSVLTVLHQDWLNGKEYKCKVSNKALPAPIEKTISKAKGQ | |
| | PREPQVYTLPPSRDELTKNQVSLTCLVKGFYPSDIAVEWESNG | |
| | QPENNYKTTPPVLDSDGSFFLYSKLTVDKSRWQQGNVFSCSVM | |
| | HEALHNHYTQKSLSLSPGK | |
| HCDR1 | gattatgcaatgcac | SEQ ID NO: 99 |
| HCDR2 | tggattaacacctacacgggcaagcccacatactcccaaaaat | SEQ ID NO: 100 |
| | tccaagga | |
| HCDR3 | gctgtattctatggatatacaatggatgcc | SEQ ID NO: 101 |
| VH | caggtccaattagtccaaagcggggcggaagtcaagaagccgg | SEQ ID NO: 102 |
| | gggcgagcgtcaaagtctcatgcaaagcgagcggatacacatt | |
| | tacggattatgcaatgcactgggtcaggcaagcacccggacaa | |
| | aggctggaatggatgggatggattaacacctacacgggcaagc | |
| | ccacatactcccaaaaattccaaggaagggtcacgataacgag | |
| | agacacgagcgcgagcaccggaatggatgggatggattaacac | |
| | ctacacgggcaagcccacatactcccaaaaattccaaggaagg | |
| | gtcacgataacgagagacacgagcgcgagcaccgtaccctggt | |
| | caccgtctcgagc | |
| LCDR1 | RASEDIYSNLA | SEQ ID NO: 4 |
| LCDR2 | SVKRLQD | SEQ ID NO: 5 |
| LCDR3 | LQGSNFPLT | SEQ ID NO: 6 |
| VL | DIQMTQSPSSLSASVGDRVTITCRASEDIYSNLAWYQQKPGKA | SEQ ID NO: 38 |
| | PKLLIFSVKRLQDGVPSRFSGSGSGTDFTLTISSLQPEDFATY | |
| | YCLQGSNFPLTFGQGTKVEIK | |
| Light chain | DIQMTQSPSSLSASVGDRVTITCRASEDIYSNLAWYQQKPGKA | SEQ ID NO: 51 |
| | PKLLIFSVKRLQDGVPSRFSGSGSGTDFTLTISSLOPEDFATY | |
| | YCLQGSNFPLTFGQGTKVEIKRTVAAPSVFIFPPSDEQLKSGT | |
| | ASVVCLLNNFYPREAKVQWKVDNALQSGNSQESVTEQDSKDST | |
| | YSLSSTLTLSKADYEKHKVYACEVTHQGLSSPVTKSENRGEC | |
| LCDR1 | agggcctccgaagacatctactccaacctggca | SEQ ID NO: 95 |
| LCDR2 | agcgtcaaaagactacaagat | SEQ ID NO: 96 |
| LCDR3 | ttgcaaggaagcaatttccccttgact | SEQ ID NO: 97 |
| VL | gacattcaaatgacgcaaagcccatcatcgctgagcgcatcgg | SEQ ID NO: 98 |
| | tcggggatagagtcaccataacatgcagggcctccgaagacat | |
| | ctactccaacctggcatggtatcaacaaaaaccggggaaggct | |
| | ccgaagctgctgatatttagcgtcaaaagactacaagatggag | |
| | taccgagccgattttcgggaagcgggagcgggacggatttcac | |
| | gctgaccatatcaagtttgcaaccggaggattttgcgacatac | |
| | tattgcttgcaaggaagcaatttccccttgactttcgggcaag | |
| | gtaccaaggtcgagatcaaa | |

hCl1c

| | | |
|---|---|---|
| HCDR1 | DYAMH | SEQ ID NO: 1 |
| HCDR2 | WINTYTGKPTYSQKFQG | SEQ ID NO: 16 |
| HCDR3 | AVFYGYTMDA | SEQ ID NO: 3 |
| VH | QVQLVQSGAEVKKPGASVKVSCKASGYTFTDYAMHWVRQAPGQ | SEQ ID NO: 34 |
| | RLEWMGWINTYTGKPTYSQKFQGRVTITRDTSASTAYMELSSL | |
| | RSEDTAVYYCARAVFYGYTMDAWGQGTLVTVSS | |
| Heavy chain | QVQLVQSGAEVKKPGASVKVSCKASGYTFTDYAMHWVRQAPGQ | SEQ ID NO: 47 |
| | RLEWMGWINTYTGKPTYSQKFQGRVTITRDTSASTAYMELSSL | |
| | RSEDTAVYYCARAVFYGYTMDAWGQGTLVTVSSASTKGPSVEP | |
| | LAPSSKSTSGGTAALGCLVKDYFPEPVTVSWNSGALTSGVHTF | |
| | PAVLQSSGLYSLSSVVTVPSSSLGTQTYICNVNHKPSNTKVDK | |
| | KVEPKSCDKTHTCPPCPAPELLGGPSVELFPPKPKDTLMISRT | |
| | PEVTCVVVDVSHEDPEVKENWYVDGVEVHNAKTKPREEQYNST | |
| | YRVVSVLTVLHQDWLNGKEYKCKVSNKALPAPIEKTISKAKGQ | |
| | PREPQVYTLPPSRDELTKNQVSLTCLVKGFYPSDIAVEWESNG | |
| | QPENNYKTTPPVLDSDGSFFLYSKLTVDKSRWQQGNVFSCSVM | |
| | HEALHNHYTQKSLSLSPGK | |
| HCDR1 | gattatgcaatgcac | SEQ ID NO: 99 |
| HCDR2 | tggattaacacctacacgggcaagcccacatactcccaaaaat | SEQ ID NO: 100 |
| | tccaagga | |
| HCDR3 | gctgtattctatggatatacaatggatgcc | SEQ ID NO: 101 |
| VH | caggtccaattagtccaaagcggggcggaagtcaagaagccgg | SEQ ID NO: 102 |
| | gggcgagcgtcaaagtctcatgcaaagcgagcggatacacatt | |
| | tacggattatgcaatgcactgggtcaggcaagcacccggacaa | |
| | aggctggaatggatgggatggattaacacctacacgggcaagc | |
| | ccacatactcccaaaaattccaaggaagggtcacgataacgag | |
| | agacacgagcgcgagcaccggaatggatgggatggattaacac | |
| | ctacacgggcaagcccacatactcccaaaaattccaaggaagg | |
| | gtcacgataacgagagacacgagcgcgagcaccgtaccctggt | |
| | caccgtctcgagc | |
| LCDR1 | RTSEDIYSNLA | SEQ ID NO: 17 |
| LCDR2 | AIKRLQD | SEQ ID NO: 14 |
| LCDR3 | LQGSKFPLT | SEQ ID NO: 11 |
| VL | DIQMTQSPSSLSASVGDRVTITCRTSEDIYSNLAWYQQKPGKA | SEQ ID NO: 39 |
| | PKLLIFAIKRLQDGVPSRFSGSGSGTDFTLTISSLOPEDFATY | |
| | YCLQGSKFPLTFGQGTKVEIK | |
| Light chain | DIQMTQSPSSLSASVGDRVTITCRTSEDIYSNLAWYQQKPGKA | SEQ ID NO: 52 |
| | PKLLIFAIKRLQDGVPSRFSGSGSGTDFTLTISSLOPEDFATY | |
| | YCLQGSKFPLTFGQGTKVEIKRTVAAPSVFIFPPSDEQLKSGT | |
| | ASVVCLLNNFYPREAKVQWKVDNALQSGNSQESVTEQDSKDST | |
| | YSLSSTLTLSKADYEKHKVYACEVTHQGLSSPVTKSENRGEC | |
| LCDR1 | cgaacgagcgaggacatatactcaaaccttgca | SEQ ID NO: 103 |
| LCDR2 | gcgataaagaggctgcaagac | SEQ ID NO: 104 |
| LCDR3 | ttgcaaggctccaaatttcccctgaca | SEQ ID NO: 105 |
| VL | gacatccaaatgactcaaagcccatcatcgctatcggcatcgg | SEQ ID NO: 106 |
| | tcggggatagagtcacgataacatgccgaacgagcgaggacat | |
| | atactcaaaccttgcatggtatcaacaaaagccggggaaggcc | |
| | ccgaagctactgatattcgcgataaagaggctgcaagacggag | |
| | ttccatcacgattttcgggatctggctcggggaccgattttac | |
| | gctgactatatcatcgctgcaaccggaagattttgcaacatac | |
| | tactgcttgcaaggctccaaatttcccctgacattcggacaag | |
| | gtaccaaggtcgagatcaaa | |

hCl1d

| | | |
|---|---|---|
| HCDR1 | DYAMH | SEQ ID NO: 1 |
| HCDR2 | WINTYTGKPTYSQKFQG | SEQ ID NO: 16 |
| HCDR3 | AVFYGYTMDA | SEQ ID NO: 3 |
| VH | QVQLVQSGAEVKKPGASVKVSCKASGYTFTDYAMHWVRQAPGQ | SEQ ID NO: 34 |
| | RLEWMGWINTYTGKPTYSQKFQGRVTITRDTSASTAYMELSSL | |
| | RSEDTAVYYCARAVFYGYTMDAWGQGTLVTVSS | |
| Heavy chain | QVQLVQSGAEVKKPGASVKVSCKASGYTFTDYAMHWVRQAPGQ | SEQ ID NO: 47 |
| | RLEWMGWINTYTGKPTYSQKFQGRVTITRDTSASTAYMELSSL | |
| | RSEDTAVYYCARAVFYGYTMDAWGQGTLVTVSSASTKGPSVEP | |
| | LAPSSKSTSGGTAALGCLVKDYFPEPVTVSWNSGALTSGVHTF | |
| | PAVLQSSGLYSLSSVVTVPSSSLGTQTYICNVNHKPSNTKVDK | |
| | KVEPKSCDKTHTCPPCPAPELLGGPSVFLFPPKPKDTLMISRT | |
| | PEVTCVVVDVSHEDPEVKENWYVDGVEVHNAKTKPREEQYNST | |
| | YRVVSVLTVLHQDWLNGKEYKCKVSNKALPAPIEKTISKAKGQ | |
| | PREPQVYTLPPSRDELTKNQVSLTCLVKGFYPSDIAVEWESNG | |
| | QPENNYKTTPPVLDSDGSFFLYSKLTVDKSRWQQGNVFSCSVM | |
| | HEALHNHYTQKSLSLSPGK | |
| HCDR1 | gattatgcaatgcac | SEQ ID NO: 99 |
| HCDR2 | tggattaacacctacacgggcaagcccacatactcccaaaaat | SEQ ID NO: 100 |
| | tccaagga | |
| HCDR3 | gctgtattctatggatatacaatggatgcc | SEQ ID NO: 101 |
| VH | caggtccaattagtccaaagcggggcggaagtcaagaagccgg | SEQ ID NO: 102 |
| | gggcgagcgtcaaagtctcatgcaaagcgagcggatacacatt | |
| | tacggattatgcaatgcactgggtcaggcaagcacccggacaa | |
| | aggctggaatggatgggatggattaacacctacacgggcaagc | |
| | ccacatactcccaaaaattccaaggaagggtcacgataacgag | |
| | agacacgagcgcgagcaccggaatggatgggatggattaacac | |
| | ctacacgggcaagcccacatactcccaaaaattccaaggaagg | |
| | gtcacgataacgagagacacgagcgcgagcaccgtaccctggt | |
| | caccgtctcgagc | |
| LCDR1 | RTSEDIYSNFA | SEQ ID NO: 18 |
| LCDR2 | SVNRLQD | SEQ ID NO: 19 |
| LCDR3 | LQGSKFPLT | SEQ ID NO: 11 |
| VL | DIQMTQSPSSLSASVGDRVTITCRTSEDIYSNFAWYQQKPGKA | SEQ ID NO: 40 |
| | PKLLIYSVNRLQDGVPSRFSGSGSGTDFTLTISSLOPEDFATY | |
| | YCLQGSKFPLTFGQGTKVEIK | |
| Light chain | DIQMTQSPSSLSASVGDRVTITCRTSEDIYSNFAWYQQKPGKA | SEQ ID NO: 53 |
| | PKLLIYSVNRLQDGVPSRFSGSGSGTDFTLTISSLOPEDFATY | |
| | YCLQGSKFPLTFGQGTKVEIKRTVAAPSVFIFPPSDEQLKSGT | |
| | ASVVCLLNNFYPREAKVQWKVDNALQSGNSQESVTEQDSKDST | |
| | YSLSSTLTLSKADYEKHKVYACEVTHQGLSSPVTKSENRGEC | |
| LCDR1 | cggacgagcgaggatatttattcgaactttgca | SEQ ID NO: 107 |
| LCDR2 | cagtcaatcggctacaagat | SEQ ID NO: 108 |
| LCDR3 | ctacaagggagcaaattcccgctgaca | SEQ ID NO: 84 |
| VL | gacatccaaatgacgcaatcaccgagctcgctgagcgcatctg | SEQ ID NO: 109 |
| | tcggggaccgtgtcacaatcacatgccggacgagcgaggatat | |
| | ttattcgaactttgcatggtatcaacaaaaaccgggcaaggct | |
| | ccgaaacttttgatttattcagtcaatcggctacaagatggcg | |
| | tcccgagccgatttagcgggagcggatcgggaaccgactttac | |
| | gctgacgatatcatcgctacaaccggaggacttcgcgacttat | |
| | tactgcctacaagggagcaaattcccgctgacattcggacaag | |
| | gtaccaaggtcgagatcaaa | |

hCl1e

| | | |
|---|---|---|
| HCDR1 | DYAMY | SEQ ID NO: 12 |
| HCDR2 | WINTYTGKPTYAQKFQG | SEQ ID NO: 15 |
| HCDR3 | AVFYGYTMDA | SEQ ID NO: 3 |
| VH | QVQLVQSGAEVKKPGASVKVSCKASGYTFTDYAMYWVRQAPGQ | SEQ ID NO: 35 |
| | RLEWMGWINTYTGKPTYAQKFQGRVTITRDTSASTAYMELSSL | |
| | RSEDTAVYYCARAVFYGYTMDAWGQGTLVTVSS | |
| Heavy chain | QVQLVQSGAEVKKPGASVKVSCKASGYTFTDYAMYWVRQAPGQ | SEQ ID NO: 48 |
| | RLEWMGWINTYTGKPTYAQKFQGRVTITRDTSASTAYMELSSL | |
| | RSEDTAVYYCARAVFYGYTMDAWGQGTLVTVSSASTKGPSVEP | |
| | LAPSSKSTSGGTAALGCLVKDYFPEPVTVSWNSGALTSGVHTE | |
| | PAVLQSSGLYSLSSVVTVPSSSLGTQTYICNVNHKPSNTKVDK | |
| | KVEPKSCDKTHTCPPCPAPELLGGPSVELFPPKPKDTLMISRT | |
| | PEVTCVVVDVSHEDPEVKENWYVDGVEVHNAKTKPREEQYNST | |
| | YRVVSVLTVLHQDWLNGKEYKCKVSNKALPAPIEKTISKAKGQ | |
| | PREPQVYTLPPSRDELTKNQVSLTCLVKGFYPSDIAVEWESNG | |
| | QPENNYKTTPPVLDSDGSFFLYSKLTVDKSRWQQGNVFSCSVM | |
| | HEALHNHYTQKSLSLSPGK | |
| HCDR1 | gattacgcaatgtac | SEQ ID NO: 110 |
| HCDR2 | tggataaatacctatacgggaaagccaacatacgcccaaaaat | SEQ ID NO: 111 |
| | tccaaggc | |
| HCDR3 | gccgtcttttatggatatacgatggacgca | SEQ ID NO: 112 |
| VH | caggtccaactggtccaatcgggggctgaagtcaaaaagccgg | SEQ ID NO: 113 |
| | gggcgagcgtcaaagtcagctgcaaagcatcgggatacacatt | |
| | tacggattacgcaatgtactgggtcaggcaagcacccggccaa | |
| | cgactggaatggatgggctggataaatacctatacgggaaagc | |
| | caacatacgcccaaaaattccaaggccgcgtcacaataacgcg | |
| | ggacacgagcgcatcgacggcttatatggaactatcatcgctg | |
| | cgatcggaagacacggcggtctattattgcgcacgcgccgtct | |
| | tttatggatatacgatggacgcatgggggcagggtaccctggt | |
| | cacggtctcgagc | |
| LCDR1 | RASEDIYSNLA | SEQ ID NO: 4 |
| LCDR2 | SVKRLQD | SEQ ID NO: 5 |
| LCDR3 | LQGSNFPLT | SEQ ID NO: 6 |
| VL | DIQMTQSPSSLSASVGDRVTITCRASEDIYSNLAWYQQKPGKA | SEQ ID NO: 38 |
| | PKLLIFSVKRLQDGVPSRFSGSGSGTDFTLTISSLOPEDFATY | |
| | YCLQGSNFPLTFGQGTKVEIK | |
| Light chain | DIQMTQSPSSLSASVGDRVTITCRASEDIYSNLAWYQQKPGKA | SEQ ID NO: 51 |
| | PKLLIFSVKRLQDGVPSRFSGSGSGTDFTLTISSLOPEDFATY | |
| | YCLQGSNFPLTFGQGTKVEIKRTVAAPSVFIFPPSDEQLKSGT | |
| | ASVVCLLNNFYPREAKVQWKVDNALQSGNSQESVTEQDSKDST | |
| | YSLSSTLTLSKADYEKHKVYACEVTHQGLSSPVTKSENRGEC | |
| LCDR1 | agggcctccgaagacatctactccaacctggca | SEQ ID NO: 95 |
| LCDR2 | agcgtcaaaagactacaagat | SEQ ID NO: 96 |
| LCDR3 | ttgcaaggaagcaatttccccttgact | SEQ ID NO: 97 |
| VL | gacattcaaatgacgcaaagcccatcatcgctgagcgcatcgg | SEQ ID NO: 98 |
| | tcggggatagagtcaccataacatgcagggcctccgaagacat | |
| | ctactccaacctggcatggtatcaacaaaaaccggggaaggct | |
| | ccgaagctgctgatatttagcgtcaaaagactacaagatggag | |
| | taccgagccgattttcgggaagcgggagcgggacggatttcac | |
| | gctgaccatatcaagtttgcaaccggaggattttgcgacatac | |
| | tattgcttgcaaggaagcaatttccccttgactttcgggcaag | |
| | gtaccaaggtcgagatcaaa | |

hCl1f

| | | |
|---|---|---|
| HCDR1 | DYAMH | SEQ ID NO: 1 |
| HCDR2 | WINAYTGKPTYAQKFQG | SEQ ID NO: 20 |
| HCDR3 | AVFYGYTMDA | SEQ ID NO: 3 |
| VH | QVQLVQSGAEVKKPGASVKVSCKASGYTFTDYAMHWVRQAPGQ | SEQ ID NO: 36 |
| | RLEWMGWINAYTGKPTYAQKFQGRVTITRDTSASTAYMELSSL | |
| | RSEDTAVYYCARAVFYGYTMDAWGQGTLVTVSS | |
| Heavy chain | QVQLVQSGAEVKKPGASVKVSCKASGYTFTDYAMHWVRQAPGQ | SEQ ID NO: 49 |
| | RLEWMGWINAYTGKPTYAQKFQGRVTITRDTSASTAYMELSSL | |
| | RSEDTAVYYCARAVFYGYTMDAWGQGTLVTVSSASTKGPSVFP | |
| | LAPSSKSTSGGTAALGCLVKDYFPEPVTVSWNSGALTSGVHTF | |
| | PAVLQSSGLYSLSSVVTVPSSSLGTQTYICNVNHKPSNTKVDK | |
| | KVEPKSCDKTHTCPPCPAPELLGGPSVELFPPKPKDTLMISRT | |
| | PEVTCVVVDVSHEDPEVKENWYVDGVEVHNAKTKPREEQYNST | |
| | YRVVSVLTVLHQDWLNGKEYKCKVSNKALPAPIEKTISKAKGQ | |
| | PREPQVYTLPPSRDELTKNQVSLTCLVKGFYPSDIAVEWESNG | |
| | QPENNYKTTPPVLDSDGSFFLYSKLTVDKSRWQQGNVFSCSVM | |
| | HEALHNHYTQKSLSLSPGK | |
| HCDR1 | gactacgcaatgcac | SEQ ID NO: 114 |
| HCDR2 | tggattaatgcctacacggggaagccgacctacgcacaaaaat | SEQ ID NO: 115 |
| | tccaagga | |
| HCDR3 | gccgtcttctatggatatacgatggatgct | SEQ ID NO: 116 |
| VH | caggtccaattggtccaaagcggggcggaggtcaagaagccgg | SEQ ID NO: 117 |
| | gggcgagcgtcaaagtctcatgcaaggcaagcggatatacatt | |
| | tacggactacgcaatgcactgggtccggcaagcccctgggcaa | |
| | cggctggaatggatgggatggattaatgcctacacggggaagc | |
| | cgacctacgcacaaaaattccaaggacgagtcacgattacgcg | |
| | ggatactagcgcgagcaccgcatatatggagctaagctcgctg | |
| | cgatctgaggataccgctgtatactactgcgcgagagccgtct | |
| | tctatggatatacgatggatgcttgggggcagggtaccctggt | |
| | cacggtctcgagc | |
| LCDR1 | RASEDIYSNLA | SEQ ID NO: 4 |
| LCDR2 | SVKRLQD | SEQ ID NO: 5 |
| LCDR3 | LQGSNFPLT | SEQ ID NO: 6 |
| VL | DIQMTQSPSSLSASVGDRVTITCRASEDIYSNLAWYQQKPGKA | SEQ ID NO: 41 |
| | PKLLIYSVKRLQDGVPSRFSGSGSGTDFTLTISSLQPEDFATY | |
| | YCLQGSNFPLTFGQGTKVEIK | |
| Light chain | DIQMTQSPSSLSASVGDRVTITCRASEDIYSNLAWYQQKPGKA | SEQ ID NO: 54 |
| | PKLLIYSVKRLQDGVPSRFSGSGSGTDFTLTISSLOPEDFATY | |
| | YCLQGSNFPLTFGQGTKVEIKRTVAAPSVFIFPPSDEQLKSGT | |
| | ASVVCLLNNFYPREAKVQWKVDNALQSGNSQESVTEQDSKDST | |
| | YSLSSTLTLSKADYEKHKVYACEVTHQGLSSPVTKSENRGEC | |
| LCDR1 | cgagcttcggaggacatctatagcaacttggct | SEQ ID NO: 118 |
| LCDR2 | agcgtcaaaaggctccaagac | SEQ ID NO: 119 |
| LCDR3 | ctacaaggctctaacttcccattgaca | SEQ ID NO: 120 |
| VL | gatatccaaatgacgcaatcaccatctagcctatcggcctctg | SEQ ID NO: 121 |
| | tgggggaccgagtcaccatcacatgccgagcttcggaggacat | |
| | ctatagcaacttggcttggtatcaacaaaagccggggaaagca | |
| | ccaaagctgctgatatatagcgtcaaaaggctccaagacggag | |
| | tcccaagccgattctcgggctccggctccgggacggattttac | |
| | gctgacaatttcgagcctgcaaccggaggactttgcaacctac | |
| | tattgcctacaaggctctaacttcccattgacatttgggcaag | |
| | gtaccaaggtcgagatcaaa | |

hCl1g

| | | |
|---|---|---|
| HCDR1 | DYAMH | SEQ ID NO: 1 |
| HCDR2 | WINAYTGKPTYAQKFQG | SEQ ID NO: 20 |
| HCDR3 | AVFYGYTMDA | SEQ ID NO: 3 |
| VH | QVQLVQSGAEVKKPGASVKVSCKASGYTFTDYAMHWVRQAPGQ | SEQ ID NO: 36 |
| | RLEWMGWINAYTGKPTYAQKFQGRVTITRDTSASTAYMELSSL | |
| | RSEDTAVYYCARAVFYGYTMDAWGQGTLVTVSS | |
| Heavy chain | QVQLVQSGAEVKKPGASVKVSCKASGYTFTDYAMHWVRQAPGQ | SEQ ID NO: 49 |
| | RLEWMGWINAYTGKPTYAQKFQGRVTITRDTSASTAYMELSSL | |
| | RSEDTAVYYCARAVFYGYTMDAWGQGTLVTVSSASTKGPSVEP | |
| | LAPSSKSTSGGTAALGCLVKDYFPEPVTVSWNSGALTSGVHTF | |
| | PAVLQSSGLYSLSSVVTVPSSSLGTQTYICNVNHKPSNTKVDK | |
| | KVEPKSCDKTHTCPPCPAPELLGGPSVELFPPKPKDTLMISRT | |
| | PEVTCVVVDVSHEDPEVKENWYVDGVEVHNAKTKPREEQYNST | |
| | YRVVSVLTVLHQDWLNGKEYKCKVSNKALPAPIEKTISKAKGQ | |
| | PREPQVYTLPPSRDELTKNQVSLTCLVKGFYPSDIAVEWESNG | |
| | QPENNYKTTPPVLDSDGSFFLYSKLTVDKSRWQQGNVFSCSVM | |
| | HEALHNHYTQKSLSLSPGK | |
| HCDR1 | gactacgcaatgcac | SEQ ID NO: 114 |
| HCDR2 | tggattaatgcctacacggggaagccgacctacgcacaaaaat | SEQ ID NO: 115 |
| | tccaagga | |
| HCDR3 | gccgtcttctatggatatacgatggatgct | SEQ ID NO: 116 |
| VH | caggtccaattggtccaaagcggggcggaggtcaagaagccgg | SEQ ID NO: 117 |
| | gggcgagcgtcaaagtctcatgcaaggcaagcggatatacatt | |
| | tacggactacgcaatgcactgggtccggcaagcccctgggcaa | |
| | cggctggaatggatgggatggattaatgcctacacggggaagc | |
| | cgacctacgcacaaaaattccaaggacgagtcacgattacgcg | |
| | ggatactagcgcgagcaccgcatatatggagctaagctcgctg | |
| | cgatctgaggataccgctgtatactactgcgcgagagccgtct | |
| | tctatggatatacgatggatgcttgggggcagggtaccctggt | |
| | cacggtctcgagc | |
| LCDR1 | RTSEDIYSNFA | SEQ ID NO: 18 |
| LCDR2 | SVNRLQD | SEQ ID NO: 19 |
| LCDR3 | LQGSKFPLT | SEQ ID NO: 11 |
| VL | DIQMTQSPSSLSASVGDRVTITCRTSEDIYSNFAWYQQKPGKA | SEQ ID NO: 40 |
| | PKLLIYSVNRLQDGVPSRFSGSGSGTDFTLTISSLOPEDFATY | |
| | YCLQGSKFPLTFGQGTKVEIK | |
| Light chain | DIQMTQSPSSLSASVGDRVTITCRTSEDIYSNFAWYQQKPGKA | SEQ ID NO: 53 |
| | PKLLIYSVNRLQDGVPSRFSGSGSGTDFTLTISSLQPEDFATY | |
| | YCLQGSKFPLTFGQGTKVEIKRTVAAPSVFIFPPSDEQLKSGT | |
| | ASVVCLLNNFYPREAKVQWKVDNALQSGNSQESVTEQDSKDST | |
| | YSLSSTLTLSKADYEKHKVYACEVTHQGLSSPVTKSENRGEC | |
| LCDR1 | cggacgagcgaggatatttattcgaactttgca | SEQ ID NO: 107 |
| LCDR2 | cagtcaatcggctacaagat | SEQ ID NO: 108 |
| LCDR3 | ctacaagggagcaaattcccgctgaca | SEQ ID NO: 84 |
| VL | gacatccaaatgacgcaatcaccgagctcgctgagcgcatctg | SEQ ID NO: 109 |
| | tcggggaccgtgtcacaatcacatgccggacgagcgaggatat | |
| | ttattcgaactttgcatggtatcaacaaaaaccgggcaaggct | |
| | ccgaaacttttgatttattcagtcaatcggctacaagatggcg | |
| | tcccgagccgatttagcgggagcggatcgggaaccgactttac | |
| | gctgacgatatcatcgctacaaccggaggacttcgcgacttat | |
| | tactgcctacaagggagcaaattcccgctgacattcggacaag | |
| | gtaccaaggtcgagatcaaa | |

hCl1h

| | | |
|---|---|---|
| HCDR1 | DYAMY | SEQ ID NO: 12 |
| HCDR2 | WINAYTGKPTYAQKFQG | SEQ ID NO: 20 |
| HCDR3 | AVYYGYTMDA | SEQ ID NO: 8 |
| VH | QVQLVQSGAEVKKPGASVKVSCKASGYTFTDYAMYWVRQAPGQ | SEQ ID NO: 37 |
| | RLEWMGWINAYTGKPTYAQKFQGRVTITRDTSASTAYMELSSL | |
| | RSEDTAVYYCARAVYYGYTMDAWGQGTLVTVSS | |
| Heavy chain | QVQLVQSGAEVKKPGASVKVSCKASGYTFTDYAMYWVRQAPGQ | SEQ ID NO: 50 |
| | RLEWMGWINAYTGKPTYAQKFQGRVTITRDTSASTAYMELSSL | |
| | RSEDTAVYYCARAVYYGYTMDAWGQGTLVTVSSASTKGPSVFP | |
| | LAPSSKSTSGGTAALGCLVKDYFPEPVTVSWNSGALTSGVHTF | |
| | PAVLQSSGLYSLSSVVTVPSSSLGTQTYICNVNHKPSNTKVDK | |
| | KVEPKSCDKTHTCPPCPAPELLGGPSVFLFPPKPKDTLMISRT | |
| | PEVTCVVVDVSHEDPEVKENWYVDGVEVHNAKTKPREEQYNST | |
| | YRVVSVLTVLHQDWLNGKEYKCKVSNKALPAPIEKTISKAKGQ | |
| | PREPQVYTLPPSRDELTKNQVSLTCLVKGFYPSDIAVEWESNG | |
| | QPENNYKTTPPVLDSDGSFFLYSKLTVDKSRWQQGNVESCSVM | |
| | HEALHNHYTQKSLSLSPGK | |
| HCDR1 | gactacgctatgtat | SEQ ID NO: 122 |
| HCDR2 | tggattaatgcctacaccgggaagccgacttatgcgcaaaaat | SEQ ID NO: 123 |
| | ttcaagga | |
| HCDR3 | gcggtctactatggatatacgatggacgca | SEQ ID NO: 124 |
| VH | caggtccaactggttcaatctggagcggaagtcaagaagcccg | SEQ ID NO: 125 |
| | gagcatccgtcaaagtctcgtgcaaggcatctggatacacatt | |
| | caccgactacgctatgtattgggtccggcaagcccccggacaa | |
| | cggctggaatggatgggatggattaatgcctacaccgggaagc | |
| | cgacttatgcgcaaaaatttcaaggaagggtcacgattacgcg | |
| | ggacacgagcgcctcaaccgcatacatggagctatcgagcctg | |
| | cgaagcgaggacaccgcggtctactactgcgcgcgggcggtct | |
| | actatggatatacgatggacgcatgggggcagggtaccctggt | |
| | cacggtctcgagc | |
| LCDR1 | RASEDIYSNLA | SEQ ID NO: 4 |
| LCDR2 | SVKRLQD | SEQ ID NO: 5 |
| LCDR3 | LQGSNFPLT | SEQ ID NO: 6 |
| VL | DIQMTQSPSSLSASVGDRVTITCRASEDIYSNLAWYQQKPGKA | SEQ ID NO: 41 |
| | PKLLIYSVKRLQDGVPSRESGSGSGTDFTLTISSLQPEDFATY | |
| | YCLQGSNFPLTFGQGTKVEIK | |
| Light chain | DIQMTQSPSSLSASVGDRVTITCRASEDIYSNLAWYQQKPGKA | SEQ ID NO: 54 |
| | PKLLIYSVKRLQDGVPSRFSGSGSGTDFTLTISSLOPEDFATY | |
| | YCLQGSNFPLTFGQGTKVEIKRTVAAPSVFIFPPSDEQLKSGT | |
| | ASVVCLLNNFYPREAKVQWKVDNALQSGNSQESVTEQDSKDST | |
| | YSLSSTLTLSKADYEKHKVYACEVTHQGLSSPVTKSENRGEC | |
| LCDR1 | cgagcttcggaggacatctatagcaacttggct | SEQ ID NO: 118 |
| LCDR2 | agcgtcaaaaggctccaagac | SEQ ID NO: 119 |
| LCDR3 | ctacaaggctctaacttcccattgaca | SEQ ID NO: 120 |
| VL | gatatccaaatgacgcaatcaccatctagcctatcggcctctg | SEQ ID NO: 121 |
| | tgggggaccgagtcaccatcacatgccgagcttcggaggacat | |
| | ctatagcaacttggcttggtatcaacaaaagccggggaaagca | |
| | ccaaagctgctgatatatagcgtcaaaaggctccaagacggag | |
| | tcccaagccgattctcgggctccggctccgggacggattttac | |
| | gctgacaatttcgagcctgcaaccggaggactttgcaacctac | |
| | tattgcctacaaggctctaacttcccattgacatttgggcaag | |
| | gtaccaaggtcgagatcaaa | |

hCl1i

| | | |
|---|---|---|
| HCDR1 | DYAMY | SEQ ID NO: 12 |
| HCDR2 | WINAYTGKPTYAQKFQG | SEQ ID NO: 20 |
| HCDR3 | AVYYGYTMDA | SEQ ID NO: 8 |
| VH | QVQLVQSGAEVKKPGASVKVSCKASGYTFTDYAMYWVRQAPGQ | SEQ ID NO: 37 |
| | RLEWMGWINAYTGKPTYAQKFQGRVTITRDTSASTAYMELSSL | |
| | RSEDTAVYYCARAVYYGYTMDAWGQGTLVTVSS | |
| Heavy chain | QVQLVQSGAEVKKPGASVKVSCKASGYTFTDYAMYWVRQAPGQ | SEQ ID NO: 50 |
| | RLEWMGWINAYTGKPTYAQKFQGRVTITRDTSASTAYMELSSL | |
| | RSEDTAVYYCARAVYYGYTMDAWGQGTLVTVSSASTKGPSVEP | |
| | LAPSSKSTSGGTAALGCLVKDYFPEPVTVSWNSGALTSGVHTF | |
| | PAVLQSSGLYSLSSVVTVPSSSLGTQTYICNVNHKPSNTKVDK | |
| | KVEPKSCDKTHTCPPCPAPELLGGPSVFLFPPKPKDTLMISRT | |
| | PEVTCVVVDVSHEDPEVKENWYVDGVEVHNAKTKPREEQYNST | |
| | YRVVSVLTVLHQDWLNGKEYKCKVSNKALPAPIEKTISKAKGQ | |
| | PREPQVYTLPPSRDELTKNQVSLTCLVKGFYPSDIAVEWESNG | |
| | QPENNYKTTPPVLDSDGSFFLYSKLTVDKSRWQQGNVFSCSVM | |
| | HEALHNHYTQKSLSLSPGK | |
| HCDR1 | gactacgctatgtat | SEQ ID NO: 122 |
| HCDR2 | tggattaatgcctacaccgggaagccgacttatgcgcaaaaat | SEQ ID NO: 123 |
| | ttcaagga | |
| HCDR3 | gcggtctactatggatatacgatggacgca | SEQ ID NO: 124 |
| VH | caggtccaactggttcaatctggagcggaagtcaagaagcccg | SEQ ID NO: 125 |
| | gagcatccgtcaaagtctcgtgcaaggcatctggatacacatt | |
| | caccgactacgctatgtattgggtccggcaagcccccggacaa | |
| | cggctggaatggatgggatggattaatgcctacaccgggaagc | |
| | cgacttatgcgcaaaaatttcaaggaagggtcacgattacgcg | |
| | ggacacgagcgcctcaaccgcatacatggagctatcgagcctg | |
| | cgaagcgaggacaccgcggtctactactgcgcgcgggcggtct | |
| | actatggatatacgatggacgcatgggggcagggtaccctggt | |
| | cacggtctcgagc | |
| LCDR1 | RASEDIYSNLA | SEQ ID NO: 4 |
| LCDR2 | SVKRLQD | SEQ ID NO: 5 |
| LCDR3 | LOGSNFPLT | SEQ ID NO: 6 |
| VL | DIQMTQSPSSLSASVGDRVTITCRASEDIYSNLAWYQQKPGKA | SEQ ID NO: 38 |
| | PKLLIFSVKRLQDGVPSRFSGSGSGTDFTLTISSLQPEDFATY | |
| | YCLQGSNFPLTFGQGTKVEIK | |
| Light chain | DIQMTQSPSSLSASVGDRVTITCRASEDIYSNLAWYQQKPGKA | SEQ ID NO: 51 |
| | PKLLIFSVKRLQDGVPSRFSGSGSGTDFTLTISSLOPEDFATY | |
| | YCLQGSNFPLTFGQGTKVEIKRTVAAPSVFIFPPSDEQLKSGT | |
| | ASVVCLLNNFYPREAKVQWKVDNALQSGNSQESVTEQDSKDST | |
| | YSLSSTLTLSKADYEKHKVYACEVTHQGLSSPVTKSENRGEC | |
| LCDR1 | agggcctccgaagacatctactccaacctggca | SEQ ID NO: 95 |
| LCDR2 | agcgtcaaaagactacaagat | SEQ ID NO: 96 |
| LCDR3 | ttgcaaggaagcaatttccccttgact | SEQ ID NO: 97 |
| VL | gacattcaaatgacgcaaagcccatcatcgctgagcgcatcgg | SEQ ID NO: 98 |
| | tcggggatagagtcaccataacatgcagggcctccgaagacat | |
| | ctactccaacctggcatggtatcaacaaaaaccggggaaggct | |
| | ccgaagctgctgatatttagcgtcaaaagactacaagatggag | |
| | taccgagccgattttcgggaagcgggagcgggacggatttcac | |
| | gctgaccatatcaagtttgcaaccggaggattttgcgacatac | |
| | tattgcttgcaaggaagcaatttccccttgactttcgggcaag | |
| | gtaccaaggtcgagatcaaa | |

hCl1j

| | | |
|---|---|---|
| HCDR1 | DYAMY | SEQ ID NO: 12 |
| HCDR2 | WINAYTGKPTYAQKFQG | SEQ ID NO: 20 |
| HCDR3 | AVYYGYTMDA | SEQ ID NO: 8 |
| VH | QVQLVQSGAEVKKPGASVKVSCKASGYTFTDYAMYWVRQAPGQ | SEQ ID NO: 37 |
| | RLEWMGWINAYTGKPTYAQKFQGRVTITRDTSASTAYMELSSL | |
| | RSEDTAVYYCARAVYYGYTMDAWGQGTLVTVSS | |
| Heavy chain | QVQLVQSGAEVKKPGASVKVSCKASGYTFTDYAMYWVRQAPGQ | SEQ ID NO: 50 |
| | RLEWMGWINAYTGKPTYAQKFQGRVTITRDTSASTAYMELSSL | |
| | RSEDTAVYYCARAVYYGYTMDAWGQGTLVTVSSASTKGPSVEP | |
| | LAPSSKSTSGGTAALGCLVKDYFPEPVTVSWNSGALTSGVHTE | |
| | PAVLQSSGLYSLSSVVTVPSSSLGTQTYICNVNHKPSNTKVDK | |
| | KVEPKSCDKTHTCPPCPAPELLGGPSVFLFPPKPKDTLMISRT | |
| | PEVTCVVVDVSHEDPEVKENWYVDGVEVHNAKTKPREEQYNST | |
| | YRVVSVLTVLHQDWLNGKEYKCKVSNKALPAPIEKTISKAKGQ | |
| | PREPQVYTLPPSRDELTKNQVSLTCLVKGFYPSDIAVEWESNG | |
| | QPENNYKTTPPVLDSDGSFFLYSKLTVDKSRWQQGNVFSCSVM | |
| | HEALHNHYTQKSLSLSPGK | |
| HCDR1 | gactacgctatgtat | SEQ ID NO: 122 |
| HCDR2 | tggattaatgcctacaccgggaagccgacttatgcgcaaaaat | SEQ ID NO: 123 |
| | ttcaagga | |
| HCDR3 | gcggtctactatggatatacgatggacgca | SEQ ID NO: 124 |
| VH | caggtccaactggttcaatctggagcggaagtcaagaagcccg | SEQ ID NO: 125 |
| | gagcatccgtcaaagtctcgtgcaaggcatctggatacacatt | |
| | caccgactacgctatgtattgggtccggcaagcccccggacaa | |
| | cggctggaatggatgggatggattaatgcctacaccgggaagc | |
| | cgacttatgcgcaaaaatttcaaggaagggtcacgattacgcg | |
| | ggacacgagcgcctcaaccgcatacatggagctatcgagcctg | |
| | cgaagcgaggacaccgcggtctactactgcgcgcgggcggtct | |
| | actatggatatacgatggacgcatgggggcagggtaccctggt | |
| | cacggtctcgagc | |
| LCDR1 | RTSEDIYSNLA | SEQ ID NO: 17 |
| LCDR2 | AIKRLQD | SEQ ID NO: 14 |
| LCDR3 | LQGSKFPLT | SEQ ID NO: 11 |
| VL | DIQMTQSPSSLSASVGDRVTITCRTSEDIYSNLAWYQQKPGKA | SEQ ID NO: 39 |
| | PKLLIFAIKRLQDGVPSRFSGSGSGTDFTLTISSLOPEDFATY | |
| | YCLQGSKFPLTFGQGTKVEIK | |
| Light chain | DIQMTQSPSSLSASVGDRVTITCRTSEDIYSNLAWYQQKPGKA | SEQ ID NO: 52 |
| | PKLLIFAIKRLQDGVPSRFSGSGSGTDFTLTISSLOPEDFATY | |
| | YCLQGSKFPLTFGQGTKVEIKRTVAAPSVFIFPPSDEQLKSGT | |
| | ASVVCLLNNFYPREAKVQWKVDNALQSGNSQESVTEQDSKDST | |
| | YSLSSTLTLSKADYEKHKVYACEVTHQGLSSPVTKSENRGEC | |
| LCDR1 | cgaacgagcgaggacatatactcaaaccttgca | SEQ ID NO: 103 |
| LCDR2 | gcgataaagaggctgcaagac | SEQ ID NO: 104 |
| LCDR3 | ttgcaaggctccaaatttcccctgaca | SEQ ID NO: 105 |
| VL | gacatccaaatgactcaaagcccatcatcgctatcggcatcgg | SEQ ID NO: 106 |
| | tcggggatagagtcacgataacatgccgaacgagcgaggacat | |
| | atactcaaaccttgcatggtatcaacaaaagccggggaaggcc | |
| | ccgaagctactgatattcgcgataaagaggctgcaagacggag | |
| | ttccatcacgattttcgggatctggctcggggaccgattttac | |
| | gctgactatatcatcgctgcaaccggaagattttgcaacatac | |
| | tactgcttgcaaggctccaaatttcccctgacattcggacaag | |
| | gtaccaaggtcgagatcaaa | |

The antibodies described in further Examples 2 to 5 were modified to contain a RLPQTGG tag (SEQ ID NO: 131) at the C-terminal end of the HC and/or a GGGGSLPQTGG tag (SEQ ID NO: 132) at the C-terminal end of the LC. The C-terminal lysine (K) on the HC was in this case replaced by the Arg (R) of the tag. The addition of the tags did not change the affinity to and specificity for CLDN18.2 of the antibodies.

### Example 2: ELISA Assay and FC Titration to Confirm the Binding to CLDN18.2 of Chimeric and Humanized Antibody Variants

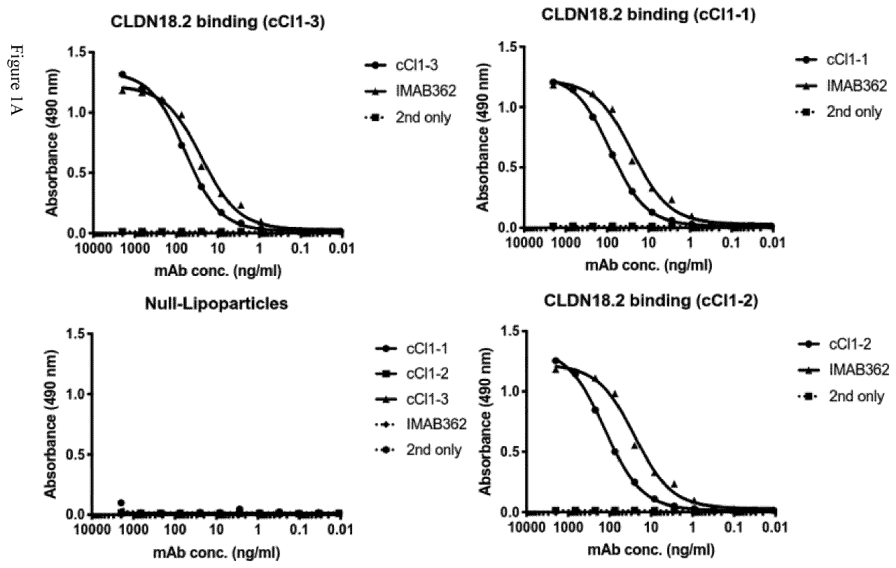

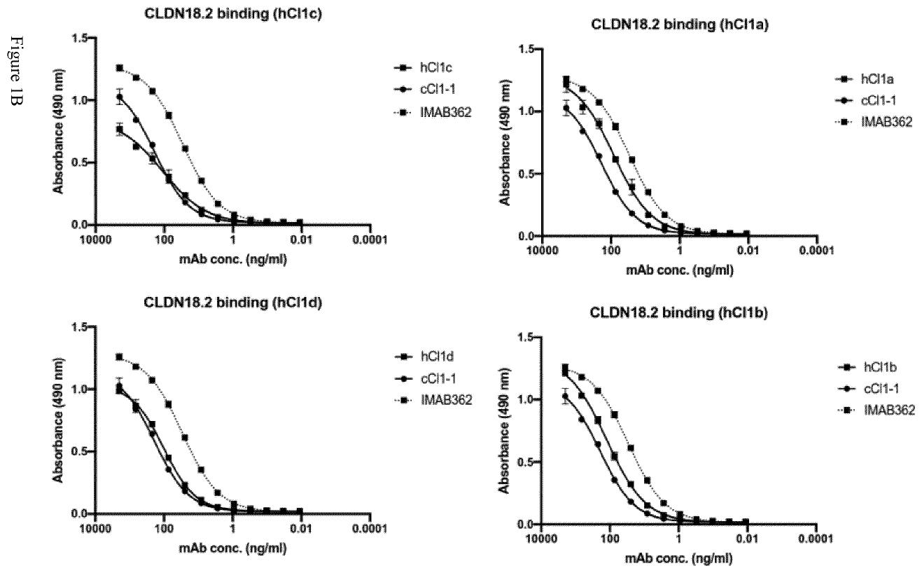

The binding affinity to CLDN18.2 of the chimeric and humanized antibodies (hCl) was tested in an ELISA assay with lipoparticles bearing CLDN18.2 as source of antigen. CLDN18.2-lipoparticles and Null-lipoparticles (without bound antigens as a negative control) were used to coat 96-well plates at a final concentration of 10 U/ml. Upon washing with PBS/0.05% Tween-(PBS-T) and blocking with PBS-T/3% BSA for at least 1 h at 37° C., 1:3 serial dilutions of the tested antibodies with a starting concentration of 2 μg/ml were added to the coated wells and incubated for at least 1 h at 37° C. The presence of bound antibodies was revealed through binding of an HRP-goat anti-human secondary antibody, development with SIGMAFAST™ OPD as peroxidase substrate and the reaction was stopped by adding 2M H₂SO₄, followed by reading the OD at 490 nm on an ELISA plate reader. Representative binding curves are shown in FIG. 1. All tested antibodies of the invention bind specifically to CLDN18.2 containing lipoparticles. Interestingly, humanization of the chimeric antibody did not result in decreased affinity as could be expected and even increased its affinity for 6 out of 10 antibodies, compared to the parental chimeric cCl1-1 antibody.

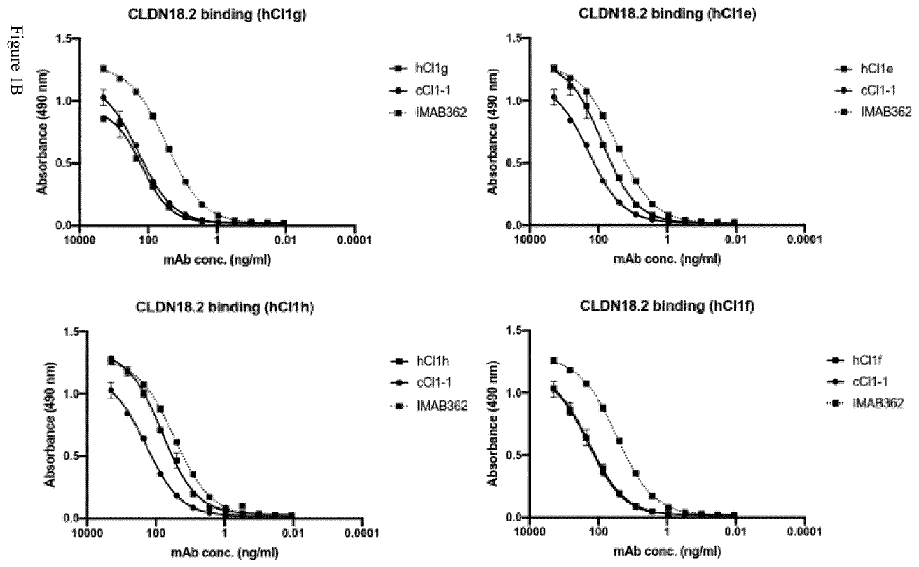

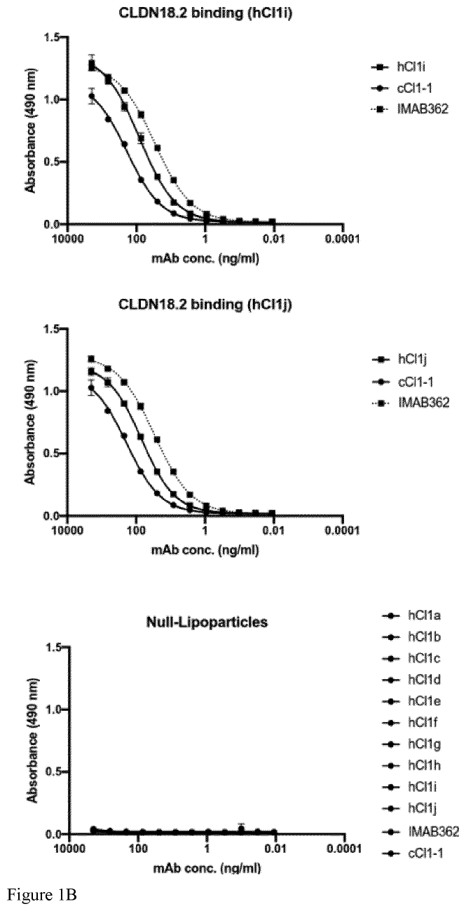

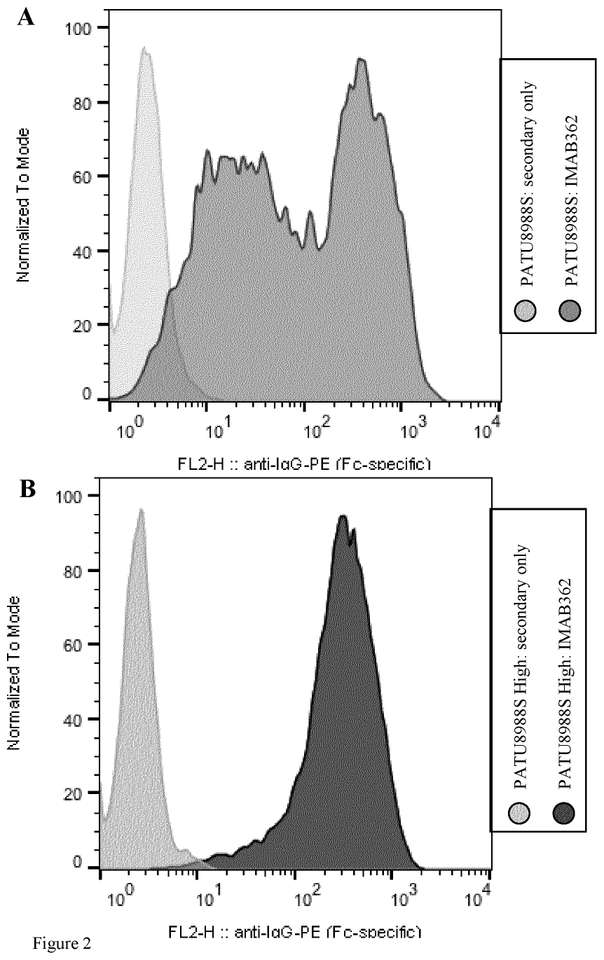

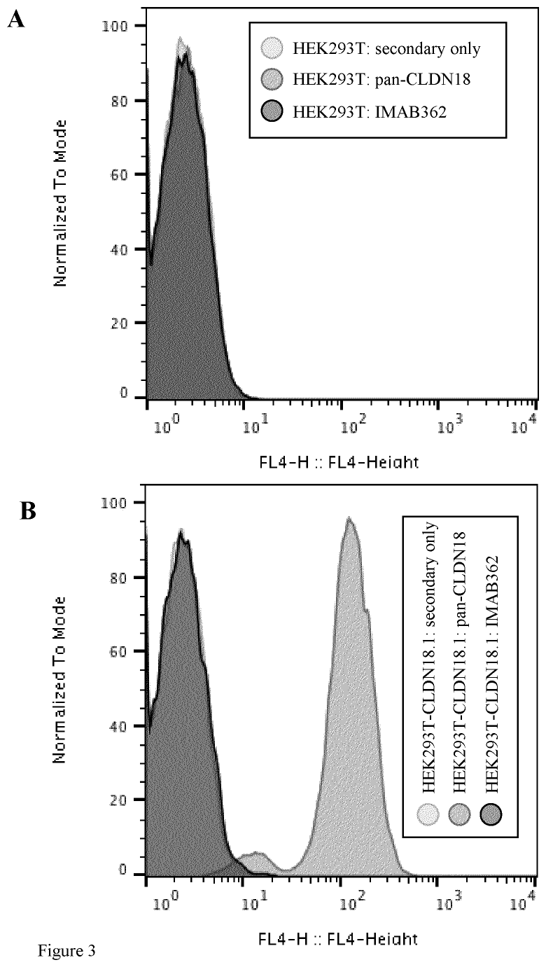

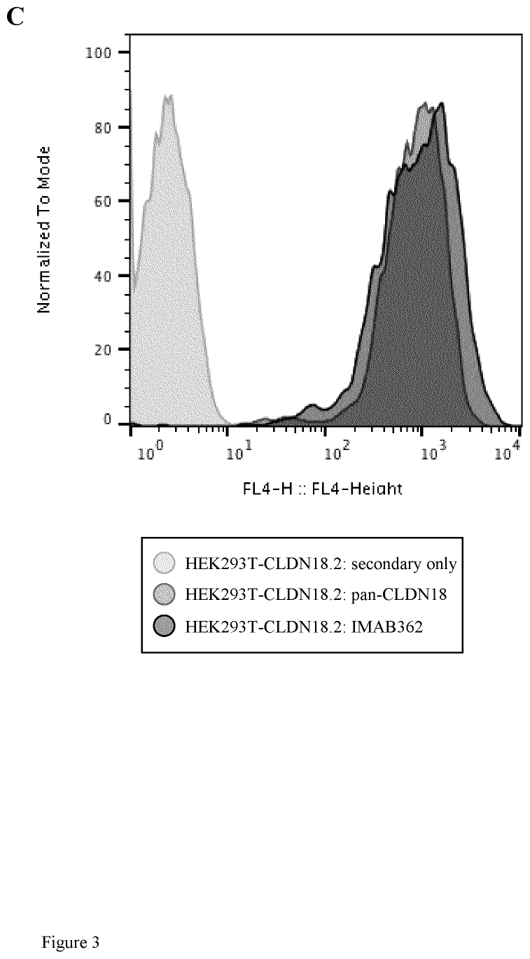

The binding of the chimeric and humanized antibodies to CLDN18.2 was also tested by FC titration with PA-TU-8988S cells (Creative Bioarray, catalog number CSC-00326) and HEK293T (ATCC, CRL-3216™) cells overexpressing CLDN18.2. FC titration allow to measure the half maximal effective concentration (EC50) of tested antibodies. PA-TU-8988S cells expressing high levels of CLDN18.2 were selected by FACS. Herein, these cells are designated as PA-TU-8988S-High cells. Based on FACS staining with IMAB362, the PA-TU-8988S cell population expresses different levels of CLDN18.2, with a high and a medium level of expression (see FIG. 2A). In order to have a more homogenous cell population, the cells were sorted by FACS to select only cells with a the higher CLDN18.2 expression. In brief, PA-TU-8988S cells suspended in FACS buffer (PBS, 2% FCS) were incubated on ice for 30 min with IMAB362 at 2 μg/ml. After wash in FACS buffer, the cells were incubated with the PE-labeled Fey specific IgG goat anti-human secondary antibody (eBioscience) on ice for 30 min. After wash, the stained cells were resuspended in FACS buffer, analyzed and sorted by a FACSAria™ instrument, separating medium expressing cells from high expressing cells (FIG. 2B). After sorting the collected PA-TU-89885-High cells were resuspended in growth media, expanded and frozen aliquots were preserved in liquid N₂. HEK293T cells overexpressing CLDN18.2 or CLDN18.1 were generated as described in Example 3 and the expression of CLDN18.2 was analyzed by flow cytometry (FIG. 3).

In order to quantify the binding of the antibodies to CLDN18.2, 250×10³ cells/well of HEK293T cells overexpressing CLDN18.2 or PA-TU-8988-High cells were seeded in FC buffer (PBS/2% FBS) into 96-well plates and allowed to settle by centrifugation. IMAB362 and the hCl antibodies to be tested were diluted at 20 μg/ml, followed by 1:4 serial dilutions and incubated with the platted cells for 30 min at 4° C. A PE-coupled secondary anti-human IgG antibody was added to the cells for additional 30 min at 4° C. after washes with the FC buffer, followed by further washes with FC buffer. The cells were then resuspended in 100 μl FC buffer and measured with a FACSCalibur™ cell analyzer (BD Biosciences, USA). The FC analysis (see FIG. 5 and Table 4) shows that the hCl antibodies have a higher EC50 value than IMAB362, although having a maxMFI value in the same range as IMAB362. The similar maxMFI values may be indicative of a similar on/off rate for IMAB362 and the hCl antibodies.

TABLE 4
Maximum MFI and EC50 (μg/ml) measured on all the hCl
and IMAB362 antibodies on the HEK293T cells lines overexpressing
CLDN18.2 and on the PA-TU-8988S-High cell lines.

| | HEK293T-CLDN18.2 | | PA-TU-8988S-High | |
|---|---|---|---|---|
| Antibody | Max MFI | EC50 (μg/ml) | Max MFI | EC50 (μg/ml) |
| IMAB362 | 1968 | 0.3878 | 1046 | 0.5082 |
| hCl1a | 1879 | 0.5976 | 1649 | 2.431 |
| hCl1b | 1859 | 0.5715 | 1724 | 1.984 |
| hCl1c | 1233 | 0.7531 | 1048 | 1.472 |
| hCl1d | 1642 | 0.5411 | 1530 | 1.933 |
| hCl1e | 1935 | 0.5583 | 1862 | 2.241 |
| hCl1f | 1721 | 0.7948 | 1602 | 2.144 |
| hCl1g | 1438 | 0.6779 | 1254 | 1.77 |
| hCl1h | 2076 | 0.4325 | 1949 | 1.75 |
| hCl1i | 2175 | 0.4437 | 2087 | 1.231 |
| hCl1j | 1848 | 0.4081 | 1705 | 1.157 |

### Example 3: Generation of Pre-B Cell L11 Cells and HEK293 T Cells Stably Expressing hCLDN18.1 and hCLDN18.2, Test of Binding Specificity of the Chimeric and Humanized Antibodies

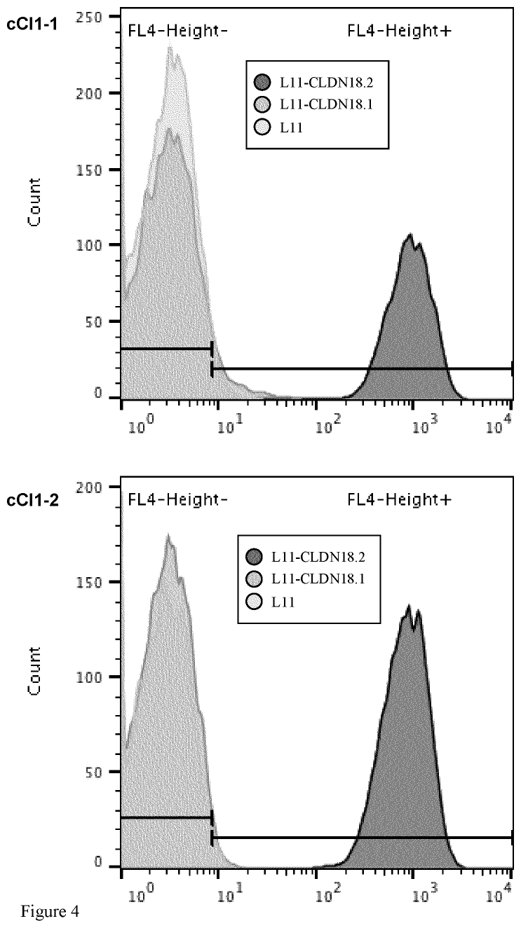

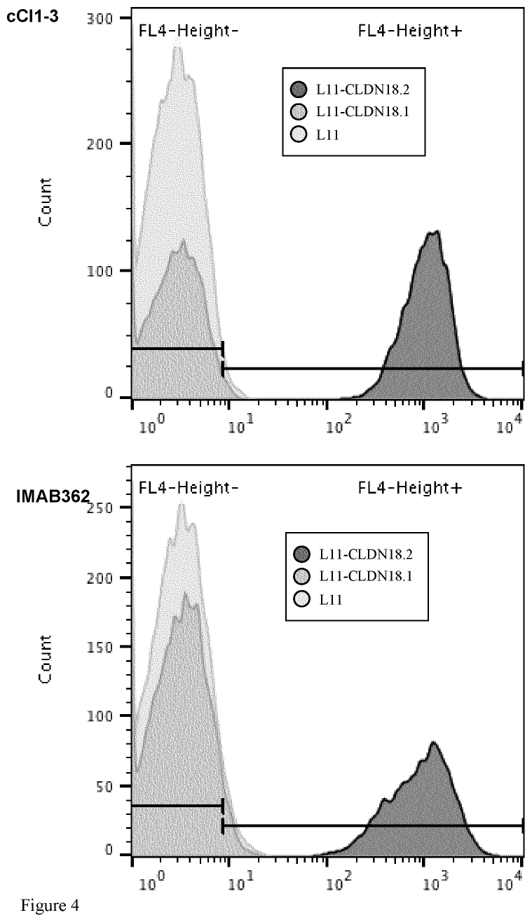

The pre-B cell L11 cell line (Waldmeier et al. 2016) and the HEK293T (ATCC CRL3216™) cell line do not endogenously express CLDN18.1 or CLDN18.2. Therefore, in order to test antibody binding, CLDN18.1 and CLDN18.2 were recombinantly overexpressed in these cell lines. Cells were co-transected by electroporation with a transposase expression construct (pcDNA3.1-by-mPB), a construct bearing transposable full-length huCLDN18.1 (pPB-Puro-huCLDN18.1) or huCLDN18.2 (pPB-Puro-huCLDN18.2) along with a puromycin resistance cassette and a construct carrying EGFP as transfection control (pEGFP-N3) (Waldmeier et al. 2016). Upon electroporation, cells were allowed to recover for two days in growth media at 37° C. in a humidified incubator in a 7.5% CO₂ atmosphere for L11 cells and 5% CO₂ atmosphere for HEK293T cells. Transfection was verified by FC analysis of the EGFP expression. Cells expressing CLDN18.1 or CLDN18.2 were then selected by the addition of puromycin into culture at 1 μg/ml, and further expanded to allow the generation of frozen stocks in FCS with 10% DMSO. The expression of CLDN18.1 and CLDN18.2 in the transfected cells was analyzed by FC (see FIG. 3). In brief, trypsinized HEK293T cells and L11 cells grown in suspension were collected by centrifugation, resuspended in PBS/2% FCS and stained for CLDN18.2 using IMAB362 as primary antibody at 2 μg/ml on ice for 30 min and, upon washing in PBS/2% FCS, stained with anti-human IgG (Fc gamma-specific) PE goat antibody (eBioscience) as secondary antibody for 30 min on ice. Upon further wash, resuspended stained cells in ice-cold FC buffer were analyzed using a FACSCalibur™ instrument (see FIG. 4 and FIG. 5). Un-transfected parental cells, not expressing CLDN18.2, were used as negative control. The expression of CLDN18.1 was analyzed in a similar fashion, using a proprietary pan-CLDN18 antibody recognizing CLDN18.1 and CLDN18.2 (see FIG. 3). Any pan-CLDN18 antibody usable for flow cytometry measurement would also be adequate such as antibody anti-Claudin-18/CLDN18 (C-term) provided by OriGene Technologies (catalog number AP50944PU-N), CLDN18 (C-Term) Rabbit pAb from MyBioSource (catalog number MB S8555451) or the CLDN18 Antibody from ProSci (catalog number 63-847).

The L11 and HEK293T cells stably expressing huCLDN18.1 and huCLDN18.2 were consequently used to test the binding specificity of the chimeric antibodies cCl1-1, cCl1-2, cCl1-3 and the humanized antibodies to CLDN18.2 and not to CLDN18.1. The cells were stained on ice for 30 min using the antibodies at 2 μg/ml and, upon washing in PBS/2% FCS, stained with anti-human IgG (Fc gamma-specific) PE goat antibody (eBioscience) as secondary antibody for 30 min on ice. All three chimeric antibodies (FIG. 4) and humanized antibodies (FIG. 5) bind to huCLDN18.2 expressed by L11 or HEK293T cells, and not to huCLDN18.1. Furthermore, the humanized antibodies bind to huCLDN18.2 with a similar affinity as IMAB362 and with an at least as good affinity as cCl1-1 (FIG. 5).

### Example 4: Testing of Humanized CLDN18.2 Antibodies Binding Activity by Flow Cytometry on Live Tumor Tissue and Live Stomach Tissue

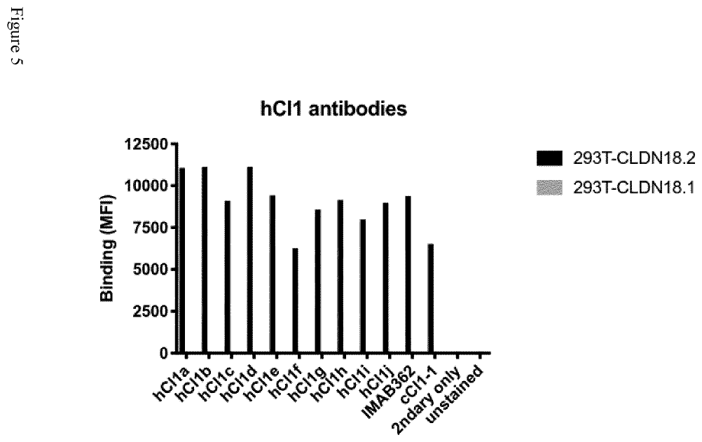

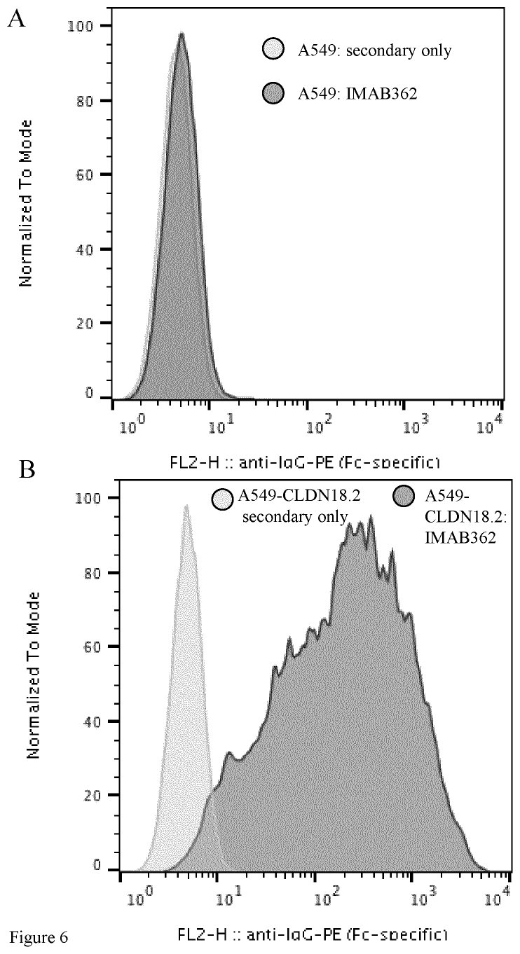

The A549 (ATCC CCL-185™) cell line does not endogenously express CLDN18.1 or CLDN18.2. In order to test antibody binding to CLDN18.2, CLDN18.2 was expressed in A549 cells. A549 cells were co-transfected by electroporation with a transposase expression construct (pcDNA3.1-hy-mPB) (Klose et al. 2017), a construct bearing transposable full-length huCldn18.2 (pPB-Puro-huCldn18.1) along with puromycin expression cassette and a construct carrying EGFP as transfection control (pEGFP-N3) (Waldmeier et al. 2016). Upon electroporation, cells were allowed to recover for two days in growth media at 37° C. in a humidified incubator in a 5% CO₂ atmosphere. Transfection was verified by FC analysis of the EGFP expression. Cells expressing CLDN18.1 or CLDN18.2 were then selected by the addition of puromycin into culture at 1 μg/ml, and further expanded to allow the generation of frozen stocks in FCS with 10% DMSO. The expression of CLDN18.2 in the transfected cells was analyzed by FC. In brief, trypsinized A549 cells were collected by centrifugation, resuspended in PBS/2% FCS and stained for CLDN18.2 using IMAB362 as primary antibody at 2 μg/ml on ice for 30 min and, upon washing in PBS/2% FCS, stained with anti-human IgG (Fc gamma-specific) PE goat antibody at 2.5 μg/ml (eBioscience) as secondary antibody for 30 min on ice. Upon further wash, resuspended stained cells in ice-cold FC buffer were analyzed using a FACSCalibur™ instrument (see FIG. 6). Un-transfected parental cells, not expressing CLDN18.2, were used as negative control. The cells were deposited on 6 Dec. 2019 at the DSMZ-Deutsche Sammlung von Mikroorganismen and Zellkulturen GmbH Inhoffenstr. 7B 38124 Braunschweig DE and are available under the accession number DSM ACC3360.

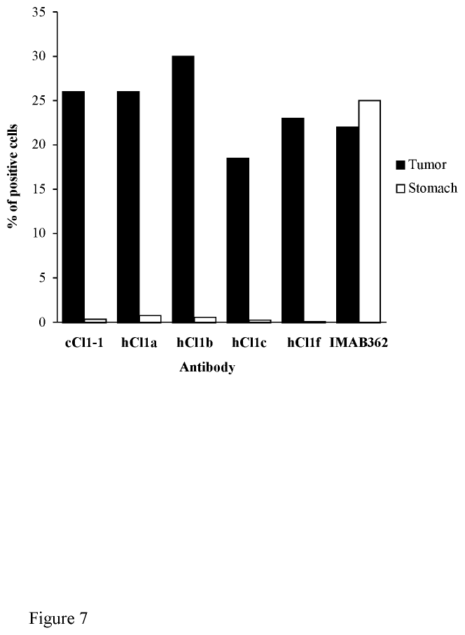

Two Balb/c mice were implanted subcutaneously with 1×10⁶ A549 cells expressing CLDN18.2 in 100 μl of 50% Matrigel and tumors growth was monitored over a few weeks until the tumor reached the desired size between 150-450 mm 3. Healthy stomach tissue and tumor tissue was collected for FC analysis. The collected tissues were cut into small pieces and digested with the Miltenyi tumor dissociation kit (MACS Miltenyi Biotec, Germany). Tissue pieces were incubated with dissociation buffer (prepared according to the manufacturer instruction) in 6 well plates for 30 min in 37° C. under permanent gentle rocking motion. Samples were resuspended and strained through a 70 μm cell strainer (Corning, USA) followed by a wash with 20 ml FC buffer (PBS+2% FBS). Cell suspensions were centrifuged (5 min at 400 g for 4° C.) and the supernatants were discarded. If needed, cell suspensions were passed through a strainer and centrifuged repeatedly and pellets resuspended in 5 ml of red blood cell lysis buffer (Biolegend, USA), incubated on ice for 4 min. After incubation, 25 ml of PBS was added, and the suspensions were centrifuged again (5 min at 400 g for 4° C.). Pellets were resuspended in FC buffer (0.5-3 ml based on pellets). Equal number of cells were transferred into 96 well plates and further processed for FC analysis. The cells in the plates were washed with PBS and centrifuged (400 g for 2 min at 4° C.). Pellets were resuspended in 50 μl/well of staining mix consisting of the antibody of choice (cCl1-1, hCl1a, hCl1b, hCl1c and hCl1f at 4 μg/ml; IMAB364 at 2 μg/ml) and the AF488-labelled AE1/AE3 pan-cytokeratin antibody (Thermo Fisher Scientific, USA) diluted in PBS and incubated for 25 min on ice. After incubation, cells were washed twice in PBS and centrifuged (400 g for 2 min at 4° C.). Pellets were resuspended in 50 μl/well of secondary staining mix (PBS+PE-labelled anti-human antibody) (Thermo Fisher Scientific, USA), and incubated 25 min on ice. After incubation cells were washed again twice in PBS. Pellets were resuspended in 100 μl of PBS containing DAPI. Plates were kept on ice until FC analysis. For FC analysis, live cells were separated from dead cells by forward scatter and DAPI stain. Live cells were then gated for the presence of cytokeratin (AF888 positive) and bound CLDN18.2 antibodies (PE positive cells). Results of the FC analysis can be seen in FIG. 7 and Table 5. The results are the average of data obtained from two mice.

All the tested antibodies (cCl1-1, hCl1a, hCl1b, hCl1c, hCl1f and IMAB364) bound to a similar percentage of tumor cells bearing CLDN18.2, approximately between 20% and 30%. However, surprisingly, only IMAB362 bound to healthy stomach cells bearing CLDN18.2 while binding of cCl1-1, hCl1a, hCl1b, hCl1c and hCl1f was barely detectable, binding less than 1% of healthy stomach cells. The difference in the binding capacity between CLDN18.2 expressed in tumor cells originating for injected A549 cells expressing CLDN18.2 and healthy stomach cells was also expressed as a ratio of the % of positive tumor cells divided by the % of positive stomach cells (see last column in Table 5). This ratio was below 5 and on average close to 1 for IMAB362, and above 15, on average above 30, for the tested humanized clones of cCl1-1 (hCl1a, hCl1b, hCl1c and hCl1f).

TABLE 5
FC binding data and binding ratio of selected antibodies
to healthy stomach cells and tumor cells.

| | % of positive tumor | | | % of positive healthy | | | | | |
|---|---|---|---|---|---|---|---|---|---|
| | cells | | | stomach cells | | | Ratio tumor/stomach | | |
| | Mouse | Mouse | | Mouse | Mouse | | Ratio | Ratio | Ratio |
| | 1 | 2 | Average | 1 | 2 | Average | Mouse 1 | Mouse 2 | Average |
| cCl1-1 | 37 | 15 | 26 | 0.4 | 0.3 | 0.35 | 92.5 | 50 | 74.3 |
| hCl1a | 34 | 18 | 26 | 1.2 | 0.3 | 0.75 | 28.3 | 60 | 34.7 |
| hCl1b | 43 | 17 | 30 | 1 | 0.13 | 0.565 | 43 | 130.7 | 53.1 |
| hCl1c | 29 | 8 | 18.5 | 0.1 | 0.4 | 0.25 | 290 | 20 | 74 |
| hCl1f | 32 | 14 | 23 | 0.04 | 0.1 | 0.07 | 800 | 140 | 328.6 |
| IMAB362 | 33 | 11 | 22 | 13 | 37 | 25 | 2.53 | 0.29 | 0.88 |

Therefore, cCl1-1 and the tested humanized clones of cCl1-1 (hCl1a, hCl1b, hCl1c and hCl1f) show increased binding to tumor cells vs. healthy stomach cells and are therefore tumor-specific CLDN18.2 antibodies. In contrast IMAB362 does not allow to discriminate tumor cells bearing CLDN18.2 form healthy stomach cells bearing CLDN18.2.

### Example 5: Testing of Humanized CLDN18.2 Antibodies by Immunohistochemistry (IHC) on Frozen Tissue Samples

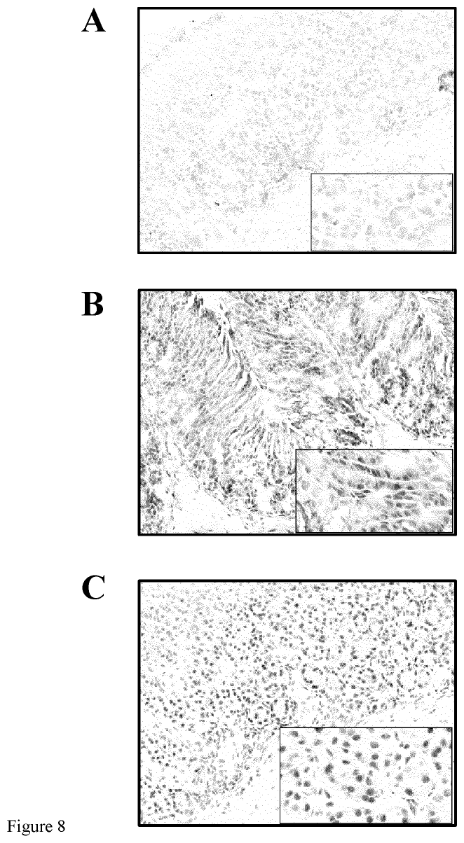

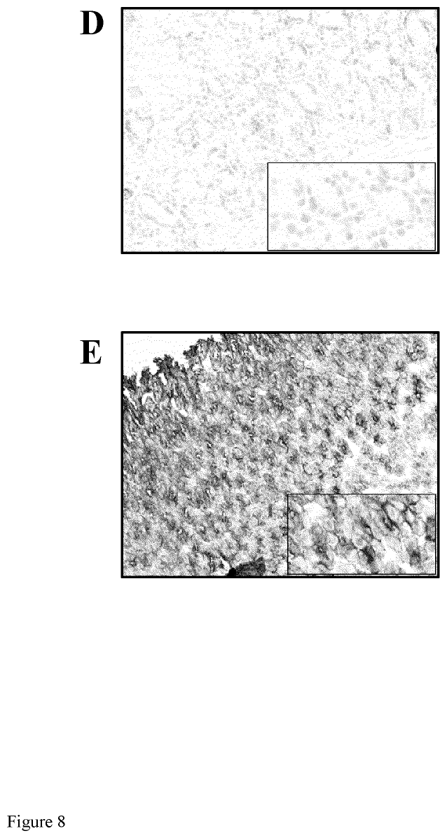

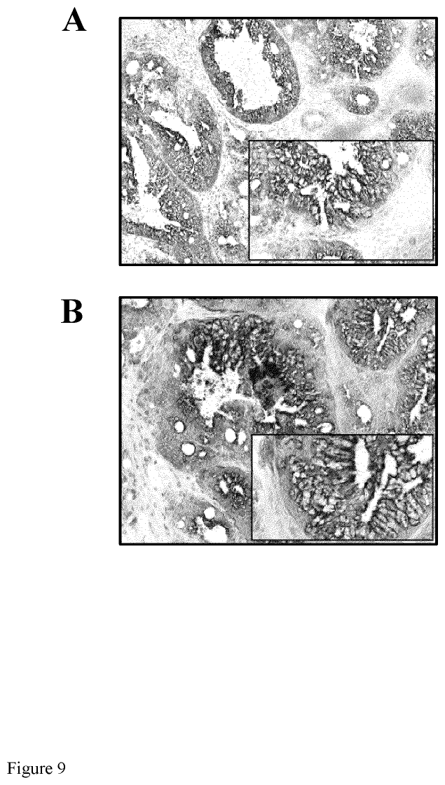

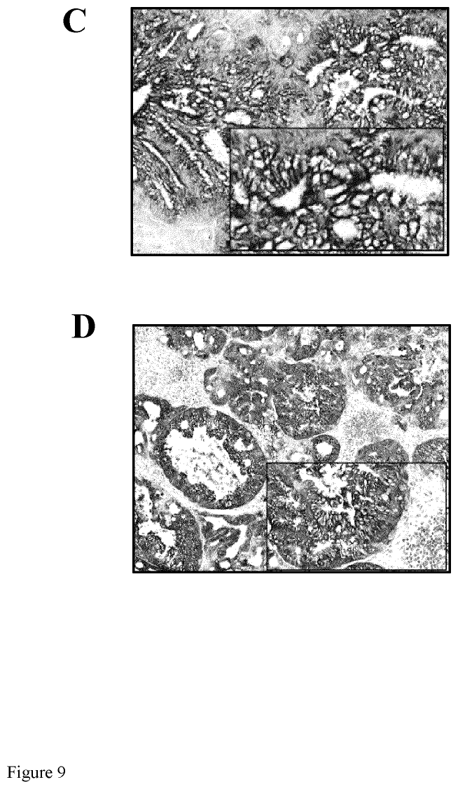

Fresh stomach and tumor tissue samples expressing CLDN18.2 obtained from Balb/c mice subcutaneously implanted with 1×10⁶ A549 cells expressing CLDN18.2 were snap-frozen in OCT in a suitable tissue mold. 5-15 μm thick tissue sections were cut with a cryostat at −20° C., transferred to microscope slide at room temperature (RT) and subsequently kept frozen until IHC staining. Before staining, slides were brought back to RT and fixed in pre-cooled acetone (−20° C.) for 10 min. After evaporation of the acetone at RT, the slides were rinsed in TBS and processed to block non-specific staining sites: slides were incubated in 0.3% H₂O₂ for 15 min at RT, followed by TBS washes and incubation in a peroxidase-blocking solution (Agilent, USA) for 60 min at RT. After blocking, the slides were processed for antibody staining: the slides were incubated with the primary antibodies (hCL1a, hCl1b, hCl1c, hCl1f, IMAB362 and the 34H14L15 pan-CLDN18 antibody (Abcam, USA)) for 120 min at RT, washed in TBS, followed by incubation with an HRP-conjugated anti-human antibody (or anti-rabbit antibody for the pan-CLDN18 antibody) for 30 min at RT. Antibody binding to CLDN18.2 or pan-CLDN18 on the tissue sections was revealed by treating the slides with the DAB+ substrate Chromogen system (Agilent, USA) according the manufacturer's instructions. After subsequent TBS washes, the slides were counterstained in hematoxylin, rinsed in dH₂O for 15 min, dehydrated in sequential 95% and 100% ethanol washes, further followed by cleaning of the slides in xylene. Finally, the slides were mounted with a coverslip in a glycerol mounting medium (Agilent, USA). Representative microscopy images of the staining of healthy mouse stomach tissue and mouse tumor tissue can be found in FIG. 8 and FIG. 9, respectively.

FIG. 8 shows representative staining of healthy stomach tissue. Only hematoxylin stain of the nuclei is visible in tissue co-stained with hCL1a, hCl1b, hCl1c and hCl1f (respectively panels A, B, C and D), while tissue stained co-stained with IMAB362 (panel E) shows membranous CLDN18.2 DAB stain. Therefore, the tested humanized clones of cCl1-1 (hCL1a, hCl1b, hCl1c and hCl1f) do not bind healthy stomach tissue expressing CLDN18.2 in contrast to IMAB362, which binds healthy stomach tissue expressing CLDN18.2. Furthermore, FIG. 9 shows representative staining of tumor tissue, panel A, B, C and D are representative image of tumor tissue stained with hCl1a, hCl1f, IMAB362 and the Abcam 34H14L15 pan-CLDN18 antibody, respectively. All the tumor stained with the tested antibodies show strong membranous CLDN18.2 DAB stain. The tested humanized clones of cCl1-1 (hCL1a and hCl1f) bound to mouse tumor tissue expressing CLDN18.2 in similarly to IMAB362 or the pan-CLDN18 antibody. Therefore, the humanized clones of cCl1-1 exhibit increased binding to tumor tissue expressing CLDN18.2 compared to heathy stomach tissue expressing CLDN18.2.

### Example 6: Asn-Deamidation and Asp-Isomerization Liability Analysis of Humanized Antibody (hCl) Variants and IMAB362

Deamidation of Asn (N) residues and isomerization of Asp (D) residues may occur during biopharmaceutical manufacturing, storage or clinical application (in vivo). Deamidation and isomerization may lead to potential changes in protein structure, function, activity, stability and immunogenicity. Therefore, it must be minimized and controlled, particularly in a regulatory context. The presence of Asn deamidation and Asp isomerization motifs can be analyzed in-silico. The most common Asn deamidation motif is the NG motif and the most common Asp-isomerization motif in the DG motif.

Such in-silico analysis revealed that all hCl antibodies had a potential DG Asp-isomerization motif in the 2^{nd} CDR of the VL, and that none of the hCl antibodies or IMAB362 had potential NG deamidation motifs in their CDRs. To verify the in-silico predictions, hCl antibodies and IMAB362 were stressed under high pH or low pH and heat to accelerate the modification that may to occur during manufacturing processes and long-term storage. In brief, antibody samples were buffer exchanged with Amicon centrifugal filters to 20 mM sodium phosphate buffer, pH 8.0 for the Asn-deamidation stress test or 20 mM citrate buffer, pH 5.5 for the Asp-isomerization stress test, and the samples were diluted to a final concentration of 3.0 mg/ml. 30 μl of sample was incubated for 1 week (Asn-deamidation) or 2 weeks (Asp-isomerization) at 40° C. in a thermoblock with a heated anti-condensation lid. The stressed and non-stressed sample was stored at −80° C. Asn-deamidation and Asp-isomerization of the samples was analyzed by strong cation exchange (SCX) chromatography. Deamidation of Asn leads in a SCX chromatogram to an increase of the peak area before the main peak (bM), while Asp-isomerization leads in a SCX chromatograph to an increase of the peak area after the main peak (aM) (Du et al. 2012). SCX chromatography was performed on a MAbPac SCX-10 Column (ThermoFisher Scientific, Basel, CH), with buffer A at pH 4.0 and buffer B at pH 11.0. The flow rate was of 0.5 ml/min with a pH gradient of 30-80% buffer B. 10 μg of the sample in 20 μl of buffer A was injected into the column. Sample detection was performed by protein absorbance at 280 nm. The hCl antibodies showed only an increase of bM of about 27.9-32.2% (see Table 6), which was not rated as critical. However, IMAB362 showed a pronounced increase in bM of 40.9% (see Table 6), even though this antibody does not have a NG motif in the variable domains. In contrast to the anti-CLDN18.2 monoclonal antibodies of the invention, IMAB362 has two NS motifs at positions HC CDR3 (aa 103-104) (SEQ ID NO: 55) and LC CDR 1 (aa 31-32) (SEQ ID NO: 56). NS motifs are the second most liable motifs for deamidation.

TABLE 6
Deamidation stress test of mAB, strong
cation exchange (SCX) chromatography

| | stressed | Proportion of | Increase proportion of bM |
|---|---|---|---|
| mAb | yes (+)/no (−) | bM (%) | after stress test (%) |
| hCl1a | − | 20.9 | 27.9 |
| | + | 48.8 | |
| hCl1b | − | 19.7 | 29.1 |
| | + | 48.8 | |
| hCl1c | − | 19.4 | 31.2 |
| | + | 50.6 | |
| hCl1d | − | 18.2 | 32.2 |
| | + | 50.4 | |
| hCl1e | − | 21.4 | 28.1 |
| | + | 49.5 | |
| hCl1f | − | 18.7 | 28.9 |
| | + | 47.6 | |
| hCl1g | − | 18.8 | 28.6 |
| | + | 47.4 | |
| hCl1h | − | 17.5 | 31.6 |
| | + | 49.1 | |
| hCl1i | − | 20.5 | 30.0 |
| | + | 50.5 | |
| hCl1j | − | 20.2 | 30.0 |
| | + | 50.2 | |
| IMAB362 | − | 26.0 | 40.9 |
| | + | 66.9 | |

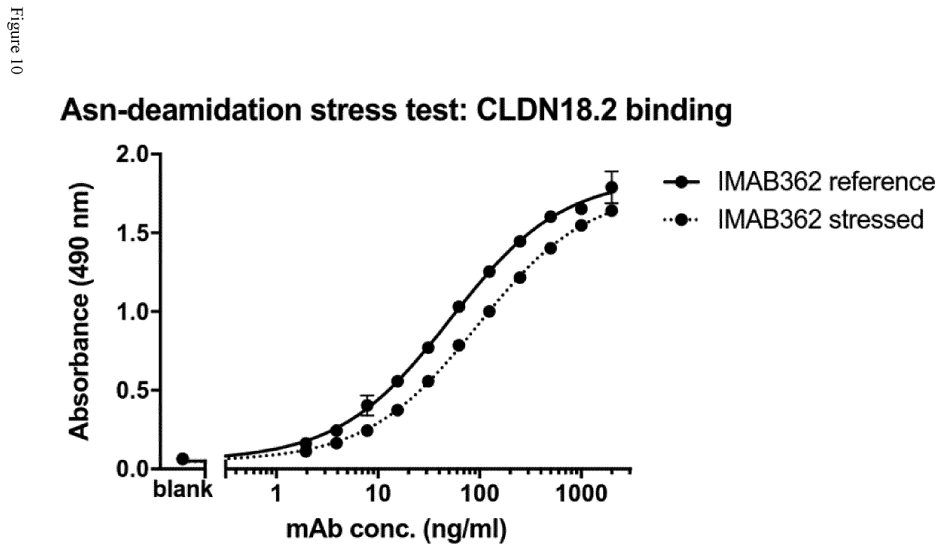

The impact of the Asn-deamidation stress test on binding affinity to CLDN18.2 of hCl1a, hCl1i and IMAB362 was tested in an ELISA assay with lipoparticles bearing CLDN18.2 as source of antigen. CLDN18.2-lipoparticles and Null-lipoparticles (without antigens) were used to coat 96-well plates at a final concentration of 10 U/ml in 100 mM sodium carbonate, pH 9.6. Upon washing with PBS/0.05% Tween-20 (PBS-T) and blocking with PBS-T/3% BSA for at least 1 h at 37° C., 1:3 serial dilutions of hCl antibodies with a starting concentration of 2 μg/ml were added and incubated for at least 1 h at 37° C. The presence of bound antibodies was revealed through binding an HRP-goat anti-human secondary antibody, developed with Sigma-Fast OPD as peroxidase substrate, the reaction was stopped by adding 2 M H₂SO₄ and reading was performed at OD-490 on an ELISA plate reader. The IMAB362 EC50 value was 1.8 times higher after the deamidation stress test (non-stressed reference: EC50 of 51.5 ng/ml, stressed: EC50 of 95.09 ng/ml) (see FIG. 10). This might be related to the increase of bM of 40.9% in SCX after deamidation stress test (see Table 6). Confirming the SCX Asn-deamidation results, no significant difference in antigen binding was observed after deamidation stress test for hCl1a and hCl1i (see Table 6). The deamidation stress test thus shows that the hCl antibodies are less prone to deamidation and potential decreased target binging than IMAB362 and predictably are more stable during manufacturing, storage and clinical application (in vivo) resulting in a more uniform and active antibody/product.

Although all hCl antibodies had a potential DG Asp-isomerization motif in the 2 nd CDR of the VL and in the CH2 and CH3 domain of the HC (VL-CDR2 (at position 62), CH2 (at position 282), CH3 (at position 403)), the Asp-isomerization stress test did not reveal Asp-isomerization (see Table 7) contrary to what could have been predicted from Du et al (Du et al. 2012). The aM values of the non-stressed samples (except for IMAB362) were already noticeably high. This may be due to lysine clipping variants of the heavy chain. IMAB362 was the only antibody without a high aM in the non-stressed sample. IMAB362 is the only tested anti-CLDN18.2 antibody without C-terminal Lys, implying that for the hCl antibodies the C-terminal Lys clipping is the most probable reason for increased aM in non-stressed and stressed samples.

TABLE 7
Asp-isomerization stress test of mAbs, strong
cation exchange (SCX) chromatography

| | stressed | Proportion of | Increase proportion of aM |
|---|---|---|---|
| mAb | yes (+)/no (−) | aM (%) | after stress test (%) |
| hCl1a | − | 45.1 | −6.5 |
| | + | 38.6 | |
| hCl1b | − | 45.2 | −5.7 |
| | + | 39.5 | |
| hCl1c | − | 40.3 | −2.3 |
| | + | 38.1 | |
| hCl1d | − | 41.3 | −4.6 |
| | + | 36.7 | |
| hCl1e | − | 44.4 | −4.2 |
| | + | 40.2 | |
| hCl1f | − | 43.5 | −1.8 |
| | + | 41.7 | |
| hCl1g | − | 44.5 | −6.4 |
| | + | 38.0 | |
| hCl1h | − | 43.2 | −4.7 |
| | + | 38.5 | |
| hCl1i | − | 44.1 | −4.6 |
| | + | 39.5 | |
| hCl1j | − | 43.7 | −7.7 |
| | + | 36.0 | |
| IMAB362 | − | 1.5 | 4.1 |
| | + | 5.6 | |

The invention is also described by the following embodiments:
1. An antibody or fragment thereof binding to CLDN18.2, wherein the antibody or fragment thereof exhibits increased binding to tumor tissue expressing CLDN18.2 over healthy tissue expressing CLDN18.2.
2. An antibody or fragment thereof binding to CLDN18.2 comprising the HCDR1, HCDR2 and HCDR3 sequences of SEQ ID NO: 21, SEQ ID NO: 22, and SEQ ID NO: 23, respectively, and the LCDR1, LCDR2 and LCDR3 sequences of SEQ ID NO: 24, SEQ ID NO: 25, and SEQ ID NO: 26, respectively.
3. The antibody or fragment thereof of embodiment 1 or 2, comprising:
   a. the HCDR1, HCDR2 and HCDR3 sequences of SEQ ID NO: 1, SEQ ID NO: 15 and SEQ ID NO: 3, respectively, and the LCDR1, LCDR2 and LCDR3 sequences of SEQ ID NO: 4, SEQ ID NO: 5 and SEQ ID NO: 6, respectively;
   b. the HCDR1, HCDR2 and HCDR3 sequences of SEQ ID NO: 1, SEQ ID NO: 16 and SEQ ID NO: 3, respectively, and the LCDR1, LCDR2 and LCDR3 sequences of SEQ ID NO: 4, SEQ ID NO: 5 and SEQ ID NO: 6, respectively;
   c. the HCDR1, HCDR2 and HCDR3 sequences of SEQ ID NO: 1, SEQ ID NO: 16 and SEQ ID NO: 3, respectively, and the LCDR1, LCDR2 and LCDR3 sequences of SEQ ID NO: 17, SEQ ID NO: 14 and SEQ ID NO: 11, respectively;
   d. the HCDR1, HCDR2 and HCDR3 sequences of SEQ ID NO: 1, SEQ ID NO: 16 and SEQ ID NO: 3, respectively, and the LCDR1, LCDR2 and LCDR3 sequences of SEQ ID NO: 18, SEQ ID NO: 19 and SEQ ID NO: 11, respectively;
   e. the HCDR1, HCDR2 and HCDR3 sequences of SEQ ID NO: 12, SEQ ID NO: 15 and SEQ ID NO: 3, respectively, and the LCDR1, LCDR2 and LCDR3 sequences of SEQ ID NO: 4, SEQ ID NO: 5 and SEQ ID NO: 6, respectively;
   f. the HCDR1, HCDR2 and HCDR3 sequences of SEQ ID NO: 1, SEQ ID NO: 20 and SEQ ID NO: 3, respectively, and the LCDR1, LCDR2 and LCDR3 sequences of SEQ ID NO: 4, SEQ ID NO: 5 and SEQ ID NO: 6, respectively;
   g. the HCDR1, HCDR2 and HCDR3 sequences of SEQ ID NO: 1, SEQ ID NO: 20 and SEQ ID NO: 3, respectively, and the LCDR1, LCDR2 and LCDR3 sequences of SEQ ID NO: 18, SEQ ID NO: 19 and SEQ ID NO: 11, respectively;
   h. the HCDR1, HCDR2 and HCDR3 sequences of SEQ ID NO: 12, SEQ ID NO: 20 and SEQ ID NO: 8, respectively, and the LCDR1, LCDR2 and LCDR3 sequences of SEQ ID NO: 4, SEQ ID NO: 5 and SEQ ID NO: 6, respectively; or
   i. the HCDR1, HCDR2 and HCDR3 sequences of SEQ ID NO: 12, SEQ ID NO: 20 and SEQ ID NO: 8, respectively, and the LCDR1, LCDR2 and LCDR3 sequences of SEQ ID NO: 17, SEQ ID NO: 14 and SEQ ID NO: 11, respectively.
4. The antibody or fragment thereof of embodiment 1 or 2, comprising:
   a. the HCDR1, HCDR2 and HCDR3 sequences of SEQ ID NO: 1, SEQ ID NO: 2 and SEQ ID NO: 3, respectively, and the LCDR1, LCDR2 and LCDR3 sequences of SEQ ID NO: 4, SEQ ID NO: 5 and SEQ ID NO: 6, respectively;
   b. the HCDR1, HCDR2 and HCDR3 sequences of SEQ ID NO: 1, SEQ ID NO: 7 and SEQ ID NO: 8, respectively, and the LCDR1, LCDR2 and LCDR3 sequences of SEQ ID NO: 9, SEQ ID NO: 10 and SEQ ID NO: 11, respectively; or
   c. the HCDR1, HCDR2 and HCDR3 sequences of SEQ ID NO: 12, SEQ ID NO: 2 and SEQ ID NO: 3, respectively, and the LCDR1, LCDR2 and LCDR3 sequences of SEQ ID NO: 13, SEQ ID NO: 14 and SEQ ID NO: 11, respectively.
5. The antibody of fragment thereof of embodiment 1 or 2, comprising:
   a. a VH sequence of SEQ ID NO: 27 and a VL sequence of SEQ ID NO: 28;
   b. a VH sequence of SEQ ID NO: 29 and a VL sequence of SEQ ID NO: 30; or
   c. a VH sequence of SEQ ID NO: 31 and a VL sequence of SEQ ID NO: 32.
6. The antibody or fragment thereof of any one of embodiments 1-3, comprising:
   a. a VH sequence having at least 80%, at least 85%, at least 90%, at least 95% or at least 98% sequence identity with the amino acid sequence of SEQ ID NO: 33;
   b. a VH sequence having at least 80%, at least 85%, at least 90%, at least 95% or at least 98% sequence identity with the amino acid sequence of SEQ ID NO: 34;
   c. a VH sequence having at least 80%, at least 85%, at least 90%, at least 95% or at least 98% sequence identity with the amino acid sequence of SEQ ID NO: 35;
   d. a VH sequence having at least 80%, at least 85%, at least 90%, at least 95% or at least 98% sequence identity with the amino acid sequence of SEQ ID NO: 36; or
   e. a VH sequence having at least 80%, at least 85%, at least 90%, at least 95% or at least 98% sequence identity with the amino acid sequence of SEQ ID NO: 37; and
   f. a VL sequence having at least 80%, at least 85%, at least 90%, at least 95% or at least 98% sequence identity with the amino acid sequence of SEQ ID NO: 38;
   g. a VL sequence having at least 80%, at least 85%, at least 90%, at least 95% or at least 98% sequence identity with the amino acid sequence of SEQ ID NO: 39;
   h. a VL sequence having at least 80%, at least 85%, at least 90%, at least 95% or at least 98% sequence identity with the amino acid sequence of SEQ ID NO: 40; or
   i. a VL sequence having at least 80%, at least 85%, at least 90%, at least 95% or at least 98% sequence identity with the amino acid sequence of SEQ ID NO: 41.
7. The antibody or fragment thereof of embodiment 1 or 2, comprising:
   a. a VH sequence of: SEQ ID NO: 33;
   b. a VH sequence of SEQ ID NO: 34;
   c. a VH sequence of SEQ ID NO: 35;
   d. a VH sequence of SEQ ID NO: 36; or
   e. a VH sequence of SEQ ID NO: 37;
      and
   f. a VL sequence of SEQ ID NO: 38;
   g. a VL sequence of SEQ ID NO: 39;
   h. a VL sequence of SEQ ID NO: 40; or
   i. a VL sequence of SEQ ID NO: 41.
8. The antibody or fragment thereof of embodiment 1 or 2, comprising:
   a. a VH sequence of SEQ ID NO: 33 and a VL sequence of SEQ ID NO: 38;
   b. a VH sequence of SEQ ID NO: 34 and a VL sequence of SEQ ID NO: 38;
   c. a VH sequence of SEQ ID NO: 34 and a VL sequence of SEQ ID NO: 39;
   d. a VH sequence of SEQ ID NO: 34 and a VL sequence of SEQ ID NO: 40;
   e. a VH sequence of SEQ ID NO: 35 and a VL sequence of SEQ ID NO: 38;
   f. a VH sequence of SEQ ID NO: 36 and a VL sequence of SEQ ID NO: 41;
   g. a VH sequence of SEQ ID NO: 36 and a VL sequence of SEQ ID NO: 40;
   h. a VH sequence of SEQ ID NO: 37 and a VL sequence of SEQ ID NO: 41;
   i. a VH sequence of SEQ ID NO: 37 and a VL sequence of SEQ ID NO: 38; or
   j. a VH sequence of SEQ ID NO: 37 and a VL sequence of SEQ ID NO: 39.
9. The antibody of any one of embodiments 1-3, comprising:
   a. a heavy chain sequence having at least 80%, at least 85%, at least 90%, at least 95% or at least 98% sequence identity with the amino acid sequence of the heavy chain sequence of SEQ ID NO: 46 and a light chain sequence having at least 80%, at least 85%, at least 90%, at least 95% or at least 98% sequence identity with the amino acid sequence of the light chain sequence of SEQ ID NO: 51;
   b. a heavy chain sequence having at least 80%, at least 85%, at least 90%, at least 95% or at least 98% sequence identity with the amino acid sequence of the heavy chain sequence of SEQ ID NO: 47 and a light chain sequence having at least 80%, at least 85%, at least 90%, at least 95% or at least 98% sequence identity with the amino acid sequence of the light chain sequence of SEQ ID NO: 51;
   c. a heavy chain sequence having at least 80%, at least 85%, at least 90%, at least 95% or at least 98% sequence identity with the amino acid sequence of the heavy chain sequence of SEQ ID NO: 47 and a light chain sequence having at least 80%, at least 85%, at least 90%, at least 95% or at least 98% sequence identity with the amino acid sequence of the light chain sequence of SEQ ID NO: 52;
   d. a heavy chain sequence having at least 80%, at least 85%, at least 90%, at least 95% or at least 98% sequence identity with the amino acid sequence of the heavy chain sequence of SEQ ID NO: 47 and a light chain sequence having at least 80%, at least 85%, at least 90%, at least 95% or at least 98% sequence identity with the amino acid sequence of the light chain sequence of SEQ ID NO: 53;
   e. a heavy chain sequence having at least 80%, at least 85%, at least 90%, at least 95% or at least 98% sequence identity with the amino acid sequence of the heavy chain sequence of SEQ ID NO: 48 and a light chain sequence having at least 80%, at least 85%, at least 90%, at least 95% or at least 98% sequence identity with the amino acid sequence of the light chain sequence of SEQ ID NO: 51;
   f. a heavy chain sequence having at least 80%, at least 85%, at least 90%, at least 95% or at least 98% sequence identity with the amino acid sequence of the heavy chain sequence of SEQ ID NO: 47 and a light chain sequence having at least 80%, at least 85%, at least 90%, at least 95% or at least 98% sequence identity with the amino acid sequence of the light chain sequence of SEQ ID NO: 54;
   g. a heavy chain sequence having at least 80%, at least 85%, at least 90%, at least 95% or at least 98% sequence identity with the amino acid sequence of the heavy chain sequence of SEQ ID NO: 49 and a light chain sequence having at least 80%, at least 85%, at least 90%, at least 95% or at least 98% sequence identity with the amino acid sequence of the light chain sequence of SEQ ID NO: 53;
   h. a heavy chain sequence having at least 80%, at least 85%, at least 90%, at least 95% or at least 98% sequence identity with the amino acid sequence of the heavy chain sequence of SEQ ID NO: 50 and a light chain sequence having at least 80%, at least 85%, at least 90%, at least 95% or at least 98% sequence identity with the amino acid sequence of the light chain sequence of SEQ ID NO: 54;
   i. a heavy chain sequence having at least 80%, at least 85%, at least 90%, at least 95% or at least 98% sequence identity with the amino acid sequence of the heavy chain sequence of SEQ ID NO: 50 and a light chain sequence having at least 80%, at least 85%, at least 90%, at least 95% or at least 98% sequence identity with the amino acid sequence of the light chain sequence of SEQ ID NO: 51;
   j. a heavy chain sequence having at least 80%, at least 85%, at least 90%, at least 95% or at least 98% sequence identity with the amino acid sequence of the heavy chain sequence of SEQ ID NO: 50 and a light chain sequence having at least 80%, at least 85%, at least 90%, at least 95% or at least 98% sequence identity with the amino acid sequence of the light chain sequence of SEQ ID NO: 52, or versions thereof with an engineered Fc domain.
The antibody of embodiment 1 or 2, comprising:
   a. the heavy chain sequence of SEQ ID NO: 46 and light chain sequence of SEQ ID NO: 51;
   b. the heavy chain sequence of SEQ ID NO: 47 and light chain sequence of SEQ ID NO: 51;
   c. the heavy chain sequence of SEQ ID NO: 47 and light chain sequence of SEQ ID NO: 52;
   d. the heavy chain sequence of SEQ ID NO: 47 and light chain sequence of SEQ ID NO: 53;
   e. the heavy chain sequence of SEQ ID NO: 48 and light chain sequence of SEQ ID NO: 51;
   f. the heavy chain sequence of SEQ ID NO: 47 and light chain sequence of SEQ ID NO: 54;
   g. the heavy chain sequence of SEQ ID NO: 49 and light chain sequence of SEQ ID NO: 53;
   h. the heavy chain sequence of SEQ ID NO: 50 and light chain sequence of SEQ ID NO: 54;
   i. the heavy chain sequence of SEQ ID NO: 50 and light chain sequence of SEQ ID NO: 51;
   j. the heavy chain sequence of SEQ ID NO: 50 and light chain sequence of SEQ ID NO: 52,
      or versions thereof with an engineered Fc domain.
11. The antibody or fragment thereof of any one of embodiments 1 to 10, wherein the antibody or fragment thereof is IgA1, IgA2, IgD, IgE, IgG1, IgG2, IgG3, IgG4, synthetic IgG, IgM, F(ab)₂, Fv, scFv, IgGACH2, F(ab′)₂, scFvCH3, Fab, VL, VH, scFv4, scFv3, scFv2, dsFv, Fv, scFv-Fc, (scFv)₂, a non-depleting IgG, a diabody, a bivalent antibody or Fc-engineered versions thereof.
12. The antibody or fragment thereof of any one of embodiments 1 to 11, wherein the antibody or fragment thereof is humanized.
13. The antibody or fragment thereof of any one of embodiments 1 to 12, wherein the antibody or fragment thereof does not bind to CLDN18.1.
14. The antibody or fragment thereof of any one of embodiments 1 to 13, wherein the antibody or fragment thereof is less susceptible to posttranslational deamidation than IMAB362.
15. The antibody or fragment thereof of any one of embodiments 1 to 14, wherein the antibody or fragment thereof labels at least 2 times more, at least 5 times more, at least 10 times more, or at least 20 times more tumor cells expressing CLDN18.2 over healthy tissue cells expressing CLDN18.2 during flow cytometry measurement.
16. The antibody or fragment thereof of any one of embodiments 1 to 14, wherein the increased binding to tumor tissue expressing CLDN18.2 over healthy tissue expressing CLDN18.2 is measured by flow cytometry or by immunohistochemistry.
17. The antibody or fragment thereof of any one of embodiments 1 to 16, wherein the antibody or fragment thereof binds to CLDN18.2 expressed in HEK293T cells or PA-TU-8988-High cells with an EC50 value that is at least 1.1 times higher, at least 1.2 times higher, at least 1.5 times higher, at least 2 times higher or at least 2.5 times higher but not more than 3 times higher than the EC50 value of IMAB362 binding to CLDN18.2 expressed in HEK293T cells or PA-TU-8988-High cells.
18. The antibody or fragment thereof of embodiment 17, wherein binding is measured by flow cytometry (FC) titration.
19. The antibody or fragment thereof of any one of embodiments 1 to 18, wherein the antibody or fragment thereof is isolated.
20. A nucleic acid encoding the antibody or fragment thereof of any of embodiments 1 to 19.
21. A vector comprising the nucleic acid of embodiment 20.
22. A host cell comprising the nucleic acid of embodiment 20 or the vector of embodiment 21.
23. The antibody or fragment thereof of any one of embodiments 1 to 19, the nucleic acid of embodiment 20, the vector of embodiment 21 or the host cell of embodiment 22 for use in the treatment of a subject
   a. suffering from,
   b. at risk of developing, and/or
   c. being diagnosed for
   a neoplastic disease.
24. The antibody or fragment thereof for the use of embodiment 23, wherein the neoplastic disease is selected from the group consisting of pancreatic, gastric, esophageal, ovarian and lung cancer.
25. An antibody or fragment thereof binding to CLDN18.2, wherein the antibody or fragment thereof
   (i) binds to the same epitope as an antibody comprising a heavy chain sequence of SEQ ID NO: 46 and a light chain sequence of SEQ ID NO: 51;
   (ii) competes for binding with an antibody comprising a heavy chain sequence of SEQ ID NO: 46 and a light chain sequence of SEQ ID NO: 51; and/or
   (iii) competitively inhibits binding of an antibody comprising a heavy chain sequence of SEQ ID NO: 46 and a light chain sequence of SEQ ID NO: 51 to CLDN18.2.

Sequences

| | |
|---|---|
| SEQ ID NO: 1 | DYAMH |
| SEQ ID NO: 2 | WINTYTGKPTYADDFKG |
| SEQ ID NO: 3 | AVFYGYTMDA |
| SEQ ID NO: 4 | RASEDIYSNLA |
| SEQ ID NO: 5 | SVKRLQD |
| SEQ ID NO: 6 | LQGSNFPLT |
| SEQ ID NO: 7 | WINAYTGKPTYADDFKG |
| SEQ ID NO: 8 | AVYYGYTMDA |
| SEQ ID NO: 9 | RTSEDIYSNFA |
| SEQ ID NO: 10 | SVNRLQD |
| SEQ ID NO: 11 | LQGSKFPLT |
| SEQ ID NO: 12 | DYAMY |
| SEQ ID NO: 13 | RTSEDIYSNLA |
| SEQ ID NO: 14 | AIKRLQD |
| SEQ ID NO: 15 | WINTYTGKPTYAQKFQG |
| SEQ ID NO: 16 | WINTYTGKPTYSQKFQG |
| SEQ ID NO: 17 | RTSEDIYSNLA |
| SEQ ID NO: 18 | RTSEDIYSNFA |
| SEQ ID NO: 19 | SVNRLQD |
| SEQ ID NO: 20 | WINAYTGKPTYAQKFQG |
| SEQ ID NO: 21 | DYAMX |
| | X in 5^{th} position is H or Y |
| SEQ ID NO: 22 | WINXYTGKPTYXXXFXG |
| | X in 4^{th} position is T or A; |
| | X in 12^{th} position is A or S; |
| | X in 13^{th} position is D or Q; |
| | X in 14^{th} position is D or K; |
| | X in 16^{th} position is K or Q |
| SEQ ID NO: 23 | AVXYGYTMDA |
| | X in 3^{rd} position is F or Y |
| SEQ ID NO: 24 | RXSEDIYSNXA |
| | X in 2^{nd} position is A or T; |
| | X in 10^{th} position is L or F |
| SEQ ID NO: 25 | XXXRLQD |
| | X in 1^{st} position is S or A; |
| | X in 2^{nd} position is V or I; |
| | X in 3^{rd} position is K or N |
| SEQ ID NO: 26 | LQGSXFPLT |
| | X in 5^{th} position is K or N |
| SEQ ID NO: 27 | cCl1-1 HC variable region |
| | QIQLVQSGPELKKPGESVKISCKASGYTFTDYAMHWVKQAPGK |
| | GLKWMGWINTYTGKPTYADDFKGRFVFSLEASASTANLQISNL |
| | KNEDTATYFCARAVFYGYTMDAWGQGTSVTVSS |
| SEQ ID NO: 28 | cCl1-1 LC variable region |
| | DIQMTQSPASLSASLGETISIACRASEDIYSNLAWYQQKSGKSPQ |
| | LLIFSVKRLQDGVPSRFSGSGSGTQYSLKISGMQPEDEGDYFCLQ |
| | GSNFPLTFGSGTKLEIK |
| SEQ ID NO: 29 | cCl1-2 HC variable region |
| | QIQLVQSGPELKKPGESVKISCKTSGYTFTDYAMHWVKQGPGK |
| | GMKWMGWINAYTGKPTYADDFKGRFVLSLEASASTANLQISN |
| | LKNEDTATYFCARAVYYGYTMDAWGQGTSVIVSS |
| SEQ ID NO: 30 | cCl1-2 LC variable region |
| | DIQMTQSPASLSASLGETISIECRTSEDIYSNFAWFQQKSGKSPQL |
| | LIYSVNRLQDGVPSRFSGSGSGTQYSLKISGMQPEDEGDYFCLQ |
| | GSKFPLTFGSGTKLEIK |
| SEQ ID NO: 31 | cCl1-3 HC variable region |
| | QIQLVQSGPELKKPGESVKISCKASGYTFTDYAMYWVKQVPGK |
| | GLRWMGWINTYTGKPTYADDFKGRFVFSLEASASTANLQISNL |
| | KNEDTATYFCARAVFYGYTMDAWGQGTSVTVSS |
| SEQ ID NO: 32 | cCl1-3 LC variable region |
| | DIQMTQSPASLSASLGETISIACRTSEDIYSNLAWYQQKSGKSPQ |
| | LLIFAIKRLQDGVPSRFSGSGSGTQYSLKISGMQPEDEGDYFCLQ |
| | GSKFPLTFGSGTKLEIK |
| SEQ ID NO: 33 | hCLla HC variable region |
| | QVQLVQSGAEVKKPGASVKVSCKASGYTFTDYAMHWVRQAP |
| | GQRLEWMGWINTYTGKPTYAQKFQGRVTITRDTSASTAYMELS |
| | SLRSEDTAVYYCARAVFYGYTMDAWGQGTLVTVSS |
| SEQ ID NO: 34 | hCL1b, c and d HC variable region |
| | QVQLVQSGAEVKKPGASVKVSCKASGYTFTDYAMHWVRQAP |
| | GQRLEWMGWINTYTGKPTYSQKFQGRVTITRDTSASTAYMELS |
| | SLRSEDTAVYYCARAVFYGYTMDAWGQGTLVTVSS |
| SEQ ID NO: 35 | hCLle HC variable region |
| | QVQLVQSGAEVKKPGASVKVSCKASGYTFTDYAMYWVRQAP |
| | GQRLEWMGWINTYTGKPTYAQKFQGRVTITRDTSASTAYMELS |
| | SLRSEDTAVYYCARAVFYGYTMDAWGQGTLVTVSS |
| SEQ ID NO: 36 | hCLIf and g HC variable region |
| | QVQLVQSGAEVKKPGASVKVSCKASGYTFTDYAMHWVRQAP |
| | GQRLEWMGWINAYTGKPTYAQKFQGRVTITRDTSASTAYMEL |
| | SSLRSEDTAVYYCARAVFYGYTMDAWGQGTLVTVSS |
| SEQ ID NO: 37 | hCL1h, i and j HC variable region |
| | QVQLVQSGAEVKKPGASVKVSCKASGYTFTDYAMYWVRQAP |
| | GQRLEWMGWINAYTGKPTYAQKFQGRVTITRDTSASTAYMEL |
| | SSLRSEDTAVYYCARAVYYGYTMDAWGQGTL VTVSS |
| SEQ ID NO: 38 | hCLla, b, e and i LC variable region |
| | DIQMTQSPSSLSASVGDRVTITCRASEDIYSNLAWYQQKPGKAP |
| | KLLIFSVKRLQDGVPSRFSGSGSGTDFTLTISSLQPEDFATYYCLQ |
| | GSNFPLTFGQGTKVEIK |
| SEQ ID NO: 39 | hCL1c and j LC variable region |
| | DIQMTQSPSSLSASVGDRVTITCRTSEDIYSNLAWYQQKPGKAP |
| | KLLIFAIKRLQDGVPSRFSGSGSGTDFTLTISSLQPEDFATYYCLQ |
| | GSKFPLTFGQGTKVEIK |
| SEQ ID NO: 40 | hCL1d and g LC variable region |
| | DIQMTQSPSSLSASVGDRVTITCRTSEDIYSNFAWYQQKPGKAP |
| | KLLIYSVNRLQDGVPSRFSGSGSGTDFTLTISSLQPEDFATYYCL |
| | QGSKFPLTFGQGTKVEIK |
| SEQ ID NO: 41 | hCLIf and h LC variable region |
| | DIQMTQSPSSLSASVGDRVTITCRASEDIYSNLAWYQQKPGKAP |
| | KLLIYSVKRLQDGVPSRFSGSGSGTDFTLTISSLQPEDFATYYCL |
| | QGSNFPLTFGQGTKVEIK |
| SEQ ID NO: 42 | hCL3a, b and c HC variable region |
| | QVQLQESGPGLVKPSETLSLTCAVSGYSVSSNYRWHWIRQPPG |
| | KGLEWIGYINIAGSTNYNPSLKSRVTISVDTSKNQFSLKLSSVTA |
| | ADTAVYYCARNPSITRAMDAWGQGTLVTVSS |
| SEQ ID NO: 43 | hCL3a LC variable region |
| | DIQMTQSPSSLSASVGDRVTITCKSSQNIFKNLEWYQQKPGKAP |
| | KLLIYYTNNLQTGVPSRFSGSGSGTDFTLTISSLQPEDFATYYCY |
| | QYNSGPFTFGQGTKVEIK |
| SEQ ID NO: 44 | hCL3b LC variable region |
| | DIQMTQSPSSLSASVGDRVTITCRSSQNIFKNLEWYQQKPGKAP |
| | KLLIYYTNNLQTGVPSRFSGSGSGTDFTLTISSLQPEDFATYYCY |
| | QYNSGPFTFGQGTKVEIK |
| SEQ ID NO: 45 | hCL3c LC variable region |
| | DIQMTQSPSSLSASVGDRVTITCRSSQNIFKNLEWYQQKPGKAP |
| | KLLIYYTNNLQSGVPSRFSGSGSGTDFTLTISSLQPEDFATYYCY |
| | QYNSGPFTFGQGTKVEIK |
| SEQ ID NO: 46 | hCLla HC full |
| | QVQLVQSGAEVKKPGASVKVSCKASGYTFTDYAMHWVRQAP |
| | GQRLEWMGWINTYTGKPTYAQKFQGRVTITRDTSASTAYMELS |
| | SLRSEDTAVYYCARAVFYGYTMDAWGQGTLVTVSSASTKGPS |
| | VFPLAPSSKSTSGGTAALGCLVKDYFPEPVTVSWNSGALTSGVH |
| | TFPAVLQSSGLYSLSSVVTVPSSSLGTQTYICNVNHKPSNTKVDK |
| | KVEPKSCDKTHTCPPCPAPELLGGPSVFLFPPKPKDTLMISRTPE |
| | VTCVVVDVSHEDPEVKFNWYVDGVEVHNAKTKPREEQYNSTY |
| | RVVSVLTVLHQDWLNGKEYKCKVSNKALPAPIEKTISKAKGQP |
| | REPQVYTLPPSRDELTKNQVSLTCLVKGFYPSDIAVEWESNGQP |
| | ENNYKTTPPVLDSDGSFFLYSKLTVDKSRWQQGNVFSCSVMHE |
| | ALHNHYTQKSLSLSPGK |
| SEQ ID NO: 47 | hCL1b, c and d HC full |
| | QVQLVQSGAEVKKPGASVKVSCKASGYTFTDYAMHWVRQAP |
| | GQRLEWMGWINTYTGKPTYSQKFQGRVTITRDTSASTAYMELS |
| | SLRSEDTAVYYCARAVFYGYTMDAWGQGTLVTVSSASTKGPS |
| | VFPLAPSSKSTSGGTAALGCLVKDYFPEPVTVSWNSGALTSGVH |
| | TFPAVLQSSGLYSLSSVVTVPSSSLGTQTYICNVNHKPSNTKVDK |
| | KVEPKSCDKTHTCPPCPAPELLGGPSVFLFPPKPKDTLMISRTPE |
| | VTCVVVDVSHEDPEVKFNWYVDGVEVHNAKTKPREEQYNSTY |
| | RVVSVLTVLHQDWLNGKEYKCKVSNKALPAPIEKTISKAKGQP |
| | REPQVYTLPPSRDELTKNQVSLTCLVKGFYPSDIAVEWESNGQP |
| | ENNYKTTPPVLDSDGSFFLYSKLTVDKSRWQQGNVFSCSVMHE |
| | ALHNHYTQKSLSLSPGK |
| SEQ ID NO: 48 | hCLle HC full |
| | QVQLVQSGAEVKKPGASVKVSCKASGYTFTDYAMYWVRQAP |
| | GQRLEWMGWINTYTGKPTYAQKFQGRVTITRDTSASTAYMELS |
| | SLRSEDTAVYYCARAVFYGYTMDAWGQGTLVTVSSASTKGPS |
| | VFPLAPSSKSTSGGTAALGCLVKDYFPEPVTVSWNSGALTSGVH |
| | TFPAVLQSSGLYSLSSVVTVPSSSLGTQTYICNVNHKPSNTKVDK |
| | KVEPKSCDKTHTCPPCPAPELLGGPSVFLFPPKPKDTLMISRTPE |
| | VTCVVVDVSHEDPEVKFNWYVDGVEVHNAKTKPREEQYNSTY |
| | RVVSVLTVLHQDWLNGKEYKCKVSNKALPAPIEKTISKAKGQP |
| | REPQVYTLPPSRDELTKNQVSLTCLVKGFYPSDIAVEWESNGQP |
| | ENNYKTTPPVLDSDGSFFLYSKLTVDKSRWQQGNVFSCSVMHE |
| | ALHNHYTQKSLSLSPGK |
| SEQ ID NO: 49 | hCLIf and g HC full |
| | QVQLVQSGAEVKKPGASVKVSCKASGYTFTDYAMHWVRQAP |
| | GQRLEWMGWINAYTGKPTYAQKFQGRVTITRDTSASTAYMEL |
| | SSLRSEDTAVYYCARAVFYGYTMDAWGQGTLVTVSSASTKGPS |
| | VFPLAPSSKSTSGGTAALGCLVKDYFPEPVTVSWNSGALTSGVH |
| | TFPAVLQSSGLYSLSSVVTVPSSSLGTQTYICNVNHKPSNTKVDK |
| | KVEPKSCDKTHTCPPCPAPELLGGPSVFLFPPKPKDTLMISRTPE |
| | VTCVVVDVSHEDPEVKFNWYVDGVEVHNAKTKPREEQYNSTY |
| | RVVSVLTVLHQDWLNGKEYKCKVSNKALPAPIEKTISKAKGQP |
| | REPQVYTLPPSRDELTKNQVSLTCLVKGFYPSDIAVEWESNGQP |
| | ENNYKTTPPVLDSDGSFFLYSKLTVDKSRWQQGNVFSCSVMHE |
| | ALHNHYTQKSLSLSPGK |
| SEQ ID NO: 50 | hCL1h, i and j HC full |
| | QVQLVQSGAEVKKPGASVKVSCKASGYTFTDYAMYWVRQAP |
| | GQRLEWMGWINAYTGKPTYAQKFQGRVTITRDTSASTAYMEL |
| | SSLRSEDTAVYYCARAVYYGYTMDAWGQGTLVTVSSASTKGP |
| | SVFPLAPSSKSTSGGTAALGCLVKDYFPEPVTVSWNSGALTSGV |
| | HTFPAVLQSSGLYSLSSVVTVPSSSLGTQTYICNVNHKPSNTKVD |
| | KKVEPKSCDKTHTCPPCPAPELLGGPSVFLFPPKPKDTLMISRTP |
| | EVTCVVVDVSHEDPEVKFNWYVDGVEVHNAKTKPREEQYNST |
| | YRVVSVLTVLHQDWLNGKEYKCKVSNKALPAPIEKTISKAKGQ |
| | PREPQVYTLPPSRDELTKNQVSLTCLVKGFYPSDIAVEWESNGQ |
| | PENNYKTTPPVLDSDGSFFLYSKLTVDKSRWQQGNVFSCSVMH |
| | EALHNHYTQKSLSLSPGK |
| SEQ ID NO: 51 | hCLla, b, e and i LC full |
| | DIQMTQSPSSLSASVGDRVTITCRASEDIYSNLAWYQQKPGKAP |
| | KLLIFSVKRLQDGVPSRFSGSGSGTDFTLTISSLQPEDFATYYCLQ |
| | GSNFPLTFGQGTKVEIKRTVAAPSVFIFPPSDEQLKSGTASVVCL |
| | LNNFYPREAKVQWKVDNALQSGNSQESVTEQDSKDSTYSLSST |
| | LTLSKADYEKHKVYACEVTHQGLSSPVTKSFNRGEC |
| SEQ ID NO: 52 | hCL1c and j LC full |
| | DIQMTQSPSSLSASVGDRVTITCRTSEDIYSNLAWYQQKPGKAP |
| | KLLIFAIKRLQDGVPSRFSGSGSGTDFTLTISSLQPEDFATYYCLQ |
| | GSKFPLTFGQGTKVEIKRTVAAPSVFIFPPSDEQLKSGTASVVCL |
| | LNNFYPREAKVQWKVDNALQSGNSQESVTEQDSKDSTYSLSST |
| | LTLSKADYEKHKVYACEVTHQGLSSPVTKSFNRGEC |
| SEQ ID NO: 53 | hCL1d and g LC full |
| | DIQMTQSPSSLSASVGDRVTITCRTSEDIYSNFAWYQQKPGKAP |
| | KLLIYSVNRLQDGVPSRFSGSGSGTDFTLTISSLQPEDFATYYCL |
| | QGSKFPLTFGQGTKVEIKRTVAAPSVFIFPPSDEQLKSGTASVVC |
| | LLNNFYPREAKVQWKVDNALQSGNSQESVTEQDSKDSTYSLSS |
| | TLTLSKADYEKHKVYACEVTHQGLSSPVTKSFNRGEC |
| SEQ ID NO: 54 | hCLIf and h LC full |
| | DIQMTQSPSSLSASVGDRVTITCRASEDIYSNLAWYQQKPGKAP |
| | KLLIYSVKRLQDGVPSRFSGSGSGTDFTLTISSLQPEDFATYYCL |
| | QGSNFPLTFGQGTKVEIKRTVAAPSVFIFPPSDEQLKSGTASVVC |
| | LLNNFYPREAKVQWKVDNALQSGNSQESVTEQDSKDSTYSLSS |
| | TLTLSKADYEKHKVYACEVTHQGLSSPVTKSFNRGEC |
| SEQ ID NO: 55 | IMAB362 HC full |
| | QVQLQQPGAELVRPGASVKLSCKASGYTFTSYWINWVKQRPG |
| | QGLEWIGNIYPSDSYTNYNQKFKDKATLTVDKSSSTAYMQLSSP |
| | TSEDSAVYYCTRSWRGNSFDYWGQGTTLTVSSASTKGPSVFPL |
| | APSSKSTSGGTAALGCLVKDYFPEPVTVSWNSGALTSGVHTFPA |
| | VLQSSGLYSLSSVVTVPSSSLGTQTYICNVNHKPSNTKVDKKVE |
| | PKSCDKTHTCPPCPAPELLGGPSVFLFPPKPKDTLMISRTPEVTC |
| | VVVDVSHEDPEVKFNWYVDGVEVHNAKTKPREEQYNSTYRVV |
| | SVLTVLHQDWLNGKEYKCKVSNKALPAPIEKTISKAKGQPREP |
| | QVYTLPPSRDELTKNQVSLTCLVKGFYPSDIAVEWESNGQPENN |
| | YKTTPPVLDSDGSFFLYSKLTVDKSRWQQGNVFSCSVMHEALH |
| | NHYTQKSLSLSPGK |
| SEQ ID NO: 56 | IMAB362 LC full |
| | DIVMTQSPSSLTVTAGEKVTMSCKSSQSLLNSGNQKNYLTWYQ |
| | QKPGQPPKLLIYWASTRESGVPDRFTGSGSGTDFTLTISSVQAED |
| | LAVYYCONDYSYPFTFGSGTKLEIKRTVAAPSVFIFPPSDEQLKS |
| | GTASVVCLLNNFYPREAKVQWKVDNALQSGNSQESVTEQDSK |
| | DSTYSLSSTLTLSKADYEKHKVYACEVTHQGLSSPVTKSFNRGE |
| | C |
| SEQ ID NO: 57 | DQWSTQDLYN |
| SEQ ID NO: 58 | NNPVTAVFNYQ |
| SEQ ID NO: 59 | STQDLYNNPVTAVF |
| SEQ ID NO: 60 | TNFWMSTANMYTG |
| SEQ ID NO: 61 | ALMIVGIVLGAIGLLV |
| SEQ ID NO: 62 | RIGSMEDSAKANMTLTSGIMFIVS |
| SEQ ID NO: 63 | METDTLLLWVLLLWVPGSTGDAAQPARRARRTKLGTELGSTPV |
| | WWNSADGRMDQWSTQDLYNNPVTAVFNYQGLWRSCVRESSG |
| | FTECRGYFTLLGLPAMLQAVRAAIQHSGGRSRRARTKTHLRRG |
| | SE |
| SEQ ID NO: 64 | MDQWSTQDLYNNPVT |
| SEQ ID NO: 65 | LYNNPVTAVFNYQGL |
| SEQ ID NO: 66 | VFNYQGLWRSCVRES |
| SEQ ID NO: 67 | QGLWRSCVRESSGFT |
| SEQ ID NO: 68 | RSCVRESSGFTECRG |
| SEQ ID NO: 69 | TEDEVQSYPSKHDYV |
| SEQ ID NO: 70 | EVQSYPSKHDYV |
| SEQ ID NO: 71 | gactacgcgatgcac |
| SEQ ID NO: 72 | tggatcaacacgtacacggggaagccgacatacgcggacgacttcaagggg |
| SEQ ID NO: 73 | gccgtcttctacggatatacgatggacgcg |
| SEQ ID NO: 74 | cagatccagctcgtccagagcgggccggagctgaagaagccgggggagagcgtgaagatctcgt |
| | gcaaggcgagcggatatacgttcacggactacgcgatgcactgggtcaagcaagcgccggggaaa |
| | gggctgaagtggatggggtggatcaacacgtacacggggaagccgacatacgcggacgacttcaa |
| | ggggcgattcgtgttctcgctggaggcgagcgcgagcacggcgaacctgcaaatctcgaacctgaa |
| | gaacgaggacacggcgacgtacttctgcgcgcgggccgtcttctacggatatacgatggacgcgtg |
| | ggggcagggtaccagcgtgacggtctcgagc |
| SEQ ID NO: 75 | cgggcgagcgaggacatctactcgaacctggcg |
| SEQ ID NO: 76 | tccgtcaagcggctgcaagac |
| SEQ ID NO: 77 | ctgcaagggagcaacttcccgctgacg |
| SEQ ID NO: 78 | gacatccagatgacgcagagcccggcgtcgctgagcgcgagcctgggggagacgatctcgatcgc |
| | gtgccgggcgagcgaggacatctactcgaacctggcgtggtatcaacagaagagcgggaagagcc |
| | cgcagctgctgatcttctccgtcaagcggctgcaagacggcgtcccgagccgattctcggggagcg |
| | ggagcgggacgcagtactcgctgaagatctcggggatgcagccggaggacgagggggactacttc |
| | tgcctgcaagggagcaacttcccgctgacgttcgggtcgggtaccaaactcgagatcaaa |
| SEQ ID NO: 79 | tggatcaacgcgtacacggggaagccgacctacgcggacgacttcaagggg |
| SEQ ID NO: 80 | gccgtctactacggatatacgatggac |
| SEQ ID NO: 81 | cagatccagctcgtccagagcgggccggagctgaagaagccgggggagagcgtgaagatctcgt |
| | gcaagacgagcggatatacgttcacggactacgcgatgcactgggtcaagcaggggccagggaaa |
| | gggatgaagtggatggggtggatcaacgcgtacacggggaagccgacctacgcggacgacttcaa |
| | ggggcgattcgtgctgagcctggaggcgagcgcctcgacggcgaacctgcaaatctcgaacctgaa |
| | gaacgaggacacggcgacgtacttctgcgcgcgggccgtctactacggatatacgatggacgcgtg |
| | ggggcagggtaccagcgtgatcgtctcgagc |
| SEQ ID NO: 82 | cggacgagcgaggacatctactcgaacttcgcg |
| SEQ ID NO: 83 | tcagtcaaccggctgcaagac |
| SEQ ID NO: 84 | ctgcaagggagcaagttcccgctgacg |
| SEQ ID NO: 85 | gacatccagatgacgcagagcccggcgagcctgagcgcgagcctgggggagacgatctcgatcg |
| | agtgccggacgagcgaggacatctactcgaacttcgcgtggttccagcagaagagcgggaagagc |
| | ccgcagctgctgatctactcagtcaaccggctgcaagacggcgtcccgagccgattctcggggagc |
| | gggagcgggacgcagtactcgctgaagatctcggggatgcagccggaggacgagggggactactt |
| | ctgcctgcaagggagcaagttcccgctgacgttcgggagcggtaccaaactcgagatcaaa |
| SEQ ID NO: 86 | gactacgcgatgtac |
| SEQ ID NO: 87 | tggatcaacacgtacacggggaagccgacctacgcggacgacttcaagggg |
| SEQ ID NO: 88 | cagatccagctcgtccagagcgggccggagctgaagaagccgggggagagcgtgaagatctcgt |
| | gcaaggcgagcggatatacgttcacggactacgcgatgtactgggtcaagcaagtgccggggaaa |
| | gggctgcgatggatggggtggatcaacacgtacacggggaagccgacctacgcggacgacttcaa |
| | ggggcgattcgtgttctcgctggaggcgagcgcgagcacggcgaacctgcaaatctcgaacctgaa |
| | gaacgaggacacggcgacgtacttctgcgcgcgggccgtcttctacggatatacgatggacgcgtg |
| | ggggcagggtaccagcgtgacggtctcgagc |
| SEQ ID NO: 89 | cggacgagcgaggacatctactcgaacctggcg |
| SEQ ID NO: 90 | gcgatcaagcggctgcaagac |
| SEQ ID NO: 91 | gacatccagatgacgcagagcccggcgagcctgagcgcgagcctgggggagacgatctcgatcg |
| | cgtgccggacgagcgaggacatctactcgaacctggcgtggtatcaacagaagagcgggaagagc |
| | ccgcagctgctgatcttcgcgatcaagcggctgcaagacggcgtcccgagccgattctcggggagc |
| | gggagcgggacgcagtactcgctgaagatctcggggatgcagccggaggacgagggggactactt |
| | ctgcctgcaagggagcaagttcccgctgacgttcgggtcgggtaccaaactcgagatcaaa |
| SEQ ID NO: 92 | tggatcaatacatacacggggaagccgacttatgcgcaaaaattccaagga |
| SEQ ID NO: 93 | gcggtcttctacggatatacgatggatgcc |
| SEQ ID NO: 94 | caggtccaactagtccaaagcggggcggaagtcaagaagcccggagcatccgtcaaagtcagctg |
| | caaggcgagcggatatacatttacggactacgcgatgcactgggtcaggcaagcccctgggcaaag |
| | gctcgaatggatgggatggatcaatacatacacggggaagccgacttatgcgcaaaaattccaagga |
| | agagtcacaattacgcgggatacatccgcatctaccgcctacatggagctaagctcgctgcggagcg |
| | aggatacggcggtctactattgcgcccgagcggtcttctacggatatacgatggatgcctgggggca |
| | gggtaccctggtcacggtctcgagc |
| SEQ ID NO: 95 | agggcctccgaagacatctactccaacctggca |
| SEQ ID NO: 96 | agcgtcaaaagactacaagat |
| SEQ ID NO: 97 | ttgcaaggaagcaatttccccttgact |
| SEQ ID NO: 98 | gacattcaaatgacgcaaagcccatcatcgctgagcgcatcggtcggggatagagtcaccataacat |
| | gcagggcctccgaagacatctactccaacctggcatggtatcaacaaaaaccggggaaggctccga |
| | agctgctgatatttagcgtcaaaagactacaagatggagtaccgagccgattttcgggaagcgggag |
| | cgggacggatttcacgctgaccatatcaagtttgcaaccggaggattttgcgacatactattgcttg |
| caa | ggaagcaatttccccttgactttcgggcaaggtaccaaggtcgagatcaaa |
| SEQ ID NO: 99 | gattatgcaatgcac |
| SEQ ID NO: 100 | tggattaacacctacacgggcaagcccacatactcccaaaaattccaagga |
| SEQ ID NO: 101 | gctgtattctatggatatacaatggatgcc |
| SEQ ID NO: 102 | caggtccaattagtccaaagcggggcggaagtcaagaagccgggggcgagcgtcaaagtctcatg |
| | caaagcgagcggatacacatttacggattatgcaatgcactgggtcaggcaagcacccggacaaag |
| | gctggaatggatgggatggattaacacctacacgggcaagcccacatactcccaaaaattccaagg |
| | aagggtcacgataacgagagacacgagcgcgagcaccggaatggatgggatggattaacacctaca |
| | cgggcaagcccacatactcccaaaaattccaaggaagggtcacgataacgagagacacgagcgcg |
| | agcaccgtaccctggtcaccgtctcgagc |
| SEQ ID NO: 103 | cgaacgagcgaggacatatactcaaaccttgca |
| SEQ ID NO: 104 | gcgataaagaggctgcaagac |
| SEQ ID NO: 105 | ttgcaaggctccaaatttcccctgaca |
| SEQ ID NO: 106 | gacatccaaatgactcaaagcccatcatcgctatcggcatcggtcggggatagagtcacgataacat |
| | gccgaacgagcgaggacatatactcaaaccttgcatggtatcaacaaaagccggggaaggccccgaa |
| | gctactgatattcgcgataaagaggctgcaagacggagttccatcacgattttcgggatctggctcg |
| | gggaccgattttacgctgactatatcatcgctgcaaccggaagattttgcaacatactactgcttgc |
| | aaggctccaaatttcccctgacattcggacaaggtaccaaggtcgagatcaaa |
| SEQ ID NO: 107 | cggacgagcgaggatatttattcgaactttgca |
| SEQ ID NO: 108 | cagtcaatcggctacaagat |
| SEQ ID NO: 109 | gacatccaaatgacgcaatcaccgagctcgctgagcgcatctgtcggggaccgtgtcacaatcacat |
| | gccggacgagcgaggatatttattcgaactttgcatggtatcaacaaaaaccgggcaaggctccgaa |
| | acttttgatttattcagtcaatcggctacaagatggcgtcccgagccgatttagcgggagcggatcggg |
| | aaccgactttacgctgacgatatcatcgctacaaccggaggacttcgcgacttattactgcctacaagg |
| | gagcaaattcccgctgacattcggacaaggtaccaaggtcgagatcaaa |
| SEQ ID NO: 110 | gattacgcaatgtac |
| SEQ ID NO: 111 | tggataaatacctatacgggaaagccaacatacgcccaaaaattccaaggc |
| SEQ ID NO: 112 | gccgtcttttatggatatacgatggacgca |
| SEQ ID NO: 113 | caggtccaactggtccaatcgggggctgaagtcaaaaagccgggggcgagcgtcaaagtcagctg |
| | caaagcatcgggatacacatttacggattacgcaatgtactgggtcaggcaagcacccggccaacga |
| | ctggaatggatgggctggataaatacctatacgggaaagccaacatacgcccaaaaattccaaggcc |
| | gcgtcacaataacgcgggacacgagcgcatcgacggcttatatggaactatcatcgctgcgatcgga |
| | agacacggcggtctattattgcgcacgcgccgtcttttatggatatacgatggacgcatgggggcagg |
| | gtaccctggtcacggtctcgagc |
| SEQ ID NO: 114 | gactacgcaatgcac |
| SEQ ID NO: 115 | tggattaatgcctacacggggaagccgacctacgcacaaaaattccaagga |
| SEQ ID NO: 116 | gccgtcttctatggatatacgatggatgct |
| SEQ ID NO: 117 | caggtccaattggtccaaagcggggcggaggtcaagaagccgggggcgagcgtcaaagtctcatg |
| | caaggcaagcggatatacatttacggactacgcaatgcactgggtccggcaagcccctgggcaacg |
| | gctggaatggatgggatggattaatgcctacacggggaagccgacctacgcacaaaaattccaagg |
| | acgagtcacgattacgcgggatactagcgcgagcaccgcatatatggagctaagctcgctgcgatct |
| | gaggataccgctgtatactactgcgcgagagccgtcttctatggatatacgatggatgcttgggggca |
| | gggtaccctggtcacggtctcgagc |
| SEQ ID NO: 118 | cgagcttcggaggacatctatagcaacttggct |
| SEQ ID NO: 119 | agcgtcaaaaggctccaagac |
| SEQ ID NO: 120 | ctacaaggctctaacttcccattgaca |
| SEQ ID NO: 121 | gatatccaaatgacgcaatcaccatctagcctatcggcctctgtgggggaccgagtcaccatcacatg |
| | ccgagcttcggaggacatctatagcaacttggcttggtatcaacaaaagccggggaaagcaccaaag |
| | ctgctgatatatagcgtcaaaaggctccaagacggagtcccaagccgattctcgggctccggctccg |
| | ggacggattttacgctgacaatttcgagcctgcaaccggaggactttgcaacctactattgcctacaag |
| | gctctaacttcccattgacatttgggcaaggtaccaaggtcgagatcaaa |
| SEQ ID NO: 122 | gactacgctatgtat |
| SEQ ID NO: 123 | tggattaatgcctacaccgggaagccgacttatgcgcaaaaatttcaagga |
| SEQ ID NO: 124 | gcggtctactatggatatacgatggacgca |
| SEQ ID NO: 125 | caggtccaactggttcaatctggagcggaagtcaagaagcccggagcatccgtcaaagtctcgtgca |
| | aggcatctggatacacattcaccgactacgctatgtattgggtccggcaagcccccggacaacggct |
| | ggaatggatgggatggattaatgcctacaccgggaagccgacttatgcgcaaaaatttcaaggaagg |
| | gtcacgattacgcgggacacgagcgcctcaaccgcatacatggagctatcgagcctgcgaagcgag |
| | gacaccgcggtctactactgcgcgcgggcggtctactatggatatacgatggacgcatgggggcag |
| | ggtaccctggtcacggtctcgagc |
| SEQ ID NO: 126 | WINXYTGKPTYXQKFQG |
| | X in 4^{th} position is T or A; |
| | X in 12^{th} position is A or S |
| | [HC CDR2 for hCl1x only, not chimeric clones cCl1-1,2,3] |
| SEQ ID NO: 127 | RTVAAPSVFIFPPSDEQLKSGTASVVCLLNNFYPREAKVQWKVD |
| | NALQSGNSQESVTEQDSKDSTYSLSSTLTLSKADYEKHKVYACE |
| | VTHQGLSSPVTKSFNRGEC [constant light chain-CL domain] |
| SEQ ID NO: 128 | ASTKGPSVFPLAPSSKSTSGGTAALGCLVKDYFPEPVTVSWNSG |
| | ALTSGVHTFPAVLQSSGLYSLSSVVTVPSSSLGTQTYICNVNHKP |
| | SNTKVDKKVEPKSCDKTHTCPPCPAPELLGGPSVFLFPPKPKDTL |
| | MISRTPEVTCVVVDVSHEDPEVKFNWYVDGVEVHNAKTKPREE |
| | QYNSTYRVVSVLTVLHQDWLNGKEYKCKVSNKALPAPIEKTIS |
| | KAKGQPREPQVYTLPPSRDELTKNQVSLTCLVKGFYPSDIAVEW |
| | ESNGQPENNYKTTPPVLDSDGSFFLYSKLTVDKSRWQQGNVFS |
| | CSVMHEALHNHYTQKSLSLSPGK [constant heavy chain-CH1 + |
| | Fc domain] |
| SEQ ID NO: 129 | ASTKGPSVFPLAPSSKSTSGGTAALGCLVKDYFPEPVTVSWNSG |
| | ALTSGVHTFPAVLQSSGLYSLSSVVTVPSSSLGTQTYICNVNHKP |
| | SNTKVDKKVEPKSCDKTHTCPPCPAPEAAGGPSVFLFPPKPKDT |
| | LMISRTPEVTCVVVDVSHEDPEVKFNWYVDGVEVHNAKTKPRE |
| | EQYNSTYRVVSVLTVLHQDWLNGKEYKCKVSNKALPAPIEKTI |
| | SKAKGQPREPQVYTLPPSRDELTKNQVSLTCLVKGFYPSDIAVE |
| | WESNGQPENNYKTTPPVLDSDGSFFLYSKLTVDKSRWQQGNVF |
| | SCSVMHEALHNHYTQKSLSLSPGK [L234A/L235A mutation in |
| | constant heavy chain-CH1 + Fc domain] |
| SEQ ID NO: 130 | ASTKGPSVFPLAPSSKSTSGGTAALGCLVKDYFPEPVTVSWNSG |
| | ALTSGVHTFPAVLQSSGLYSLSSVVTVPSSSLGTQTYICNVNHKP |
| | SNTKVDKKVEPKSCDKTHTCPPCPAPEAAGGPSVFLFPPKPKDT |
| | LMISRTPEVTCVVVDVSHEDPEVKFNWYVDGVEVHNAKTKPR |
| | EEQYNSTYRVVSVLTVLHQDWLNGKEYKCKVSNKALGAPIEK |
| | TISKAKGQPREPQVYTLPPSRDELTKNQVSLTCLVKGFYPSDIAV |
| | EWESNGQPENNYKTTPPVLDSDGSFFLYSKLTVDKSRWQQGNV |
| | FSCSVMHEALHNHYTQKSLSLSPGK [L236A/L236A/P329G |
| | mutation in constant heavy chain-CH1 + Fc domain] |
| SEQ ID NO: 131 | RLPQTGG [sortase tag] |
| SEQ ID NO: 132 | GGGGS-LPQTGG [sortase tag] |
| SEQ ID NO: 133 | CLDN18.2 |
| | MAVTACQGLGFVVSLIGIAGIIAATCMDQWSTQDLYNNPVTAV |
| | FNYQGLWRSCVRESSGFTECRGYFTLLGLPAMLQAVRALMIVG |
| | IVLGAIGLLVSIFALKCIRIGSMEDSAKANMTLTSGIMFIVSGLCA |
| | IAGVSVFANMLVTNFWMSTANMYTGMGGMVQTVQTRYTFGA |
| | ALFVGWVAGGLTLIGGVMMCIACRGLAPEETNYKAVSYHASG |
| | HSVAYKPGGFKASTGFGSNTKNKKIYDGGARTEDEVQSYPSKH |
| | DYV |

### REFERENCES

Abbott, W. M., M. M. Damschroder, and D. C. Lowe. 2014. ‘Current approaches to fine mapping of antigen-antibody interactions’, Immunology, 142: 526-35.
Abdiche, Y. N., D. S. Malashock, A. Pinkerton, and J. Pons. 2009. ‘Exploring blocking assays using Octet, ProteOn, and Biacore biosensors’, Anal Biochem, 386: 172-80.
Alegre, M. L., A. M. Collins, V. L. Pulito, R. A. Brosius, W. C. Olson, R. A. Zivin, R. Knowles, J. R. Thistlethwaite, L. K. Jolliffe, and J. A. Bluestone. 1992. ‘Effect of a single amino acid mutation on the activating and immunosuppressive properties of a “humanized” OKT3 monoclonal antibody’, J Immunol, 148: 3461-8.
An, Z., G. Forrest, R. Moore, M. Cukan, P. Haytko, L. Huang, S. Vitelli, J. Z. Zhao, P. Lu, J. Hua, C. R. Gibson, B. R. Harvey, D. Montgomery, D. Zaller, F. Wang, and W. Strohl. 2009. ‘IgG2m4, an engineered antibody isotype with reduced Fc function’, MAbs, 1: 572-9.
Bolt, S., E. Routledge, I. Lloyd, L. Chatenoud, H. Pope, S. D. Gorman, M. Clark, and H. Waldmann. 1993. ‘The generation of a humanized, non-mitogenic CD3 monoclonal antibody which retains in vitro immunosuppressive properties’, Eur J Immunol, 23: 403-11.
Chang, Z. L., and Y. Y. Chen. 2017. ‘CARs: Synthetic Immunoreceptors for Cancer Therapy and Beyond’, Trends Mol Med, 23: 430-50.
Chu, S. Y., I. Vostiar, S. Karki, G. L. Moore, G. A. Lazar, E. Pong, P. F. Joyce, D. E. Szymkowski, and J. R. Desjarlais. 2008. ‘Inhibition of B cell receptor-mediated activation of primary human B cells by coengagement of CD19 and FcgammaRIIb with Fc-engineered antibodies’, Mol Immunol, 45: 3926-33.
Dall'Acqua, W. F., R. M. Woods, E. S. Ward, S. R. Palaszynski, N. K. Patel, Y. A. Brewah, H. Wu, P. A. Kiener, and S. Langermann. 2002. ‘Increasing the affinity of a human IgG1 for the neonatal Fc receptor: biological consequences’, J Immunol, 169: 5171-80.
Diebolder, C. A., F. J. Beurskens, R. N. de Jong, R. I. Koning, K. Strumane, M. A. Lindorfer, M. Voorhorst, D. Ugurlar, S. Rosati, A. J. Heck, J. G. van de Winkel, I. A. Wilson, A. J. Koster, R. P. Taylor, E. O. Saphire, D. R. Burton, J. Schuurman, P. Gros, and P. W. Parren. 2014. ‘Complement is activated by IgG hexamers assembled at the cell surface’, Science, 343: 1260-3.
Du, Y., A. Walsh, R. Ehrick, W. Xu, K. May, and H. Liu. 2012. ‘Chromatographic analysis of the acidic and basic species of recombinant monoclonal antibodies’, MAbs, 4: 578-85.
Ellerman, D. 2019. ‘Bispecific T-cell engagers: Towards understanding variables influencing the in vitro potency and tumor selectivity and their modulation to enhance their efficacy and safety’, Methods, 154: 102-17.
Gervais, D. 2016. ‘Protein deamidation in biopharmaceutical manufacture: understanding, control and impact’, J Chem Technol Biotechnol, 91: 569-75.
Green, M. R., and J. Sambrook. 2012. Molecular Cloning: A Laboratory Manual (Fourth Edition) (Cold Spring Harbor Laboratory Press).
Hansel, T. T., H. Kropshofer, T. Singer, J. A. Mitchell, and A. J. George. 2010. ‘The safety and side effects of monoclonal antibodies’, Nat Rev Drug Discov, 9: 325-38.
Hashimoto, Y., W. Zhou, K. Hamauchi, K. Shirakura, T. Doi, K. Yagi, T. Sawasaki, Y. Okada, M. Kondoh, and H. Takeda. 2018. ‘Engineered membrane protein antigens successfully induce antibodies against extracellular regions of claudin-5’, Sci Rep, 8: 8383.
Hewitt, K. J., R. Agarwal, and P. J. Morin. 2006. ‘The claudin gene family: expression in normal and neoplastic tissues’, BMC Cancer, 6: 186.
Idusogie, E. E., P. Y. Wong, L. G. Presta, H. Gazzano-Santoro, K. Totpal, M. Ultsch, and M. G. Mulkerrin. 2001. ‘Engineered antibodies with increased activity to recruit complement’, J Immunol, 166: 2571-5.
Jacobi, A., B. Enenkel, P. Garidel, C. Eckermann, M. Knappenberger, I. Presser, and H. Kaufmann. 2014. ‘Process Development and Manufacturing of Therapeutic Antibodies.’ in S. Duebel and J. M. Reichert (eds.), Handbook of Therapeutic Antibodies, Second Edition (Wiley-VCH Verlag GmbH & Co. KGaA).
Jiang, H., Z. Shi, P. Wang, C. Wang, L. Yang, G. Du, H. Zhang, B. Shi, J. Jia, Q. Li, H. Wang, and Z. Li. 2018. ‘Claudin18.2-Specific Chimeric Antigen Receptor Engineered T Cells for the Treatment of Gastric Cancer’, J Natl Cancer Inst.
June, C. H., and M. Sadelain. 2018. ‘Chimeric Antigen Receptor Therapy’, N Engl J Med, 379: 64-73.
Klose, D., M. Woitok, J. Niesen, R. R. Beerli, U. Grawunder, R. Fischer, S. Barth, R. Fendel, and T. Nachreiner. 2017. ‘Generation of an artificial human B cell line test system using Transpo-mAb™ technology to evaluate the therapeutic efficacy of novel antigen-specific fusion proteins’, PLoS One, 12: e0180305.
Lazar, G. A., W. Dang, S. Karki, O. Vafa, J. S. Peng, L. Hyun, C. Chan, H. S. Chung, A. Eivazi, S. C. Yoder, J. Vielmetter, D. F. Carmichael, R. J. Hayes, and B. I. Dahiyat. 2006. ‘Engineered antibody Fc variants with enhanced effector function’, Proc Natl Acad Sci USA, 103: 4005-10.
Leabman, M. K., Y. G. Meng, R. F. Kelley, L. E. DeForge, K. J. Cowan, and S. Iyer. 2013. ‘Effects of altered FcgammaR binding on antibody pharmacokinetics in cynomolgus monkeys’, MAbs, 5: 896-903.
Lo, M., H. S. Kim, R. K. Tong, T. W. Bainbridge, J. M. Vernes, Y. Zhang, Y. L. Lin, S. Chung, M. S. Dennis, Y. J. Zuchero, R. J. Watts, J. A. Couch, Y. G. Meng, J. K. Atwal, R. J. Brerski, C. Spiess, and J. A. Ernst. 2017. ‘Effector-attenuating Substitutions That Maintain Antibody Stability and Reduce Toxicity in Mice’, J Biol Chem, 292: 3900-08.
Lu, X., R. P. Nobrega, H. Lynaugh, T. Jain, K. Barlow, T. Boland, A. Sivasubramanian, M. Vasquez, and Y. Xu. 2019. ‘Deamidation and isomerization liability analysis of 131 clinical-stage antibodies’, MAbs, 11: 45-57.
Martin, A. C. R., and J. Allemn. 2014. ‘Bioinformatics Tools for Analysis of Antibodies.’ in, Hanbook of Therapeutic Antibodies, Second Edition (Wiley-VCH Verlag GmbH & Co. KGaA).
Mimoto, F., T. Igawa, T. Kuramochi, H. Katada, S. Kadono, T. Kamikawa, M. Shida-Kawazoe, and K. Hattori. 2013. ‘Novel asymmetrically engineered antibody Fc variant with superior FcgammaR binding affinity and specificity compared with afucosylated Fc variant’, MAbs, 5: 229-36.
Moldenhauer, G. 2014. ‘Selection Strategies for Monoclonal Antibodies.’ in S. Duebel and J. M. Reichert (eds.), Handbook of Therapeutic Antibodies, Second Edition (Wiley-VCH Verlag GmbH & Co. KGaA).
Moore, G. L., H. Chen, S. Karki, and G. A. Lazar. 2010. ‘Engineered Fc variant antibodies with enhanced ability to recruit complement and mediate effector functions’, MAbs, 2: 181-9.
Natsume, A., M. In, H. Takamura, T. Nakagawa, Y. Shimizu, K. Kitajima, M. Wakitani, S. Ohta, M. Satoh, K. Shitara, and R. Niwa. 2008. ‘Engineered antibodies of IgG1/IgG3 mixed isotype with enhanced cytotoxic activities’, Cancer Res, 68: 3863-72.
Niimi, T., K. Nagashima, J. M. Ward, P. Minoo, D. B. Zimonjic, N. C. Popescu, and S. Kimura. 2001. ‘claudin-18, a novel downstream target gene for the T/EBP/NKX2.1 homeodomain transcription factor, encodes lung- and stomach-specific isoforms through alternative splicing’, Mol Cell Biol, 21: 7380-90.
Richards, J. O., S. Karki, G. A. Lazar, H. Chen, W. Dang, and J. R. Desjarlais. 2008. ‘Optimization of antibody binding to FcgammaRIIa enhances macrophage phagocytosis of tumor cells’, Mol Cancer Ther, 7: 2517-27.
Rother, R. P., S. A. Rollins, C. F. Mojcik, R. A. Brodsky, and L. Bell. 2007. ‘Discovery and development of the complement inhibitor eculizumab for the treatment of paroxysmal nocturnal hemoglobinuria’, Nat Biotechnol, 25: 1256-64.
Sahin, U., M. Koslowski, K. Dhaene, D. Usener, G. Brandenburg, G. Seitz, C. Huber, and O. Tureci. 2008. ‘Claudin-18 splice variant 2 is a pan-cancer target suitable for therapeutic antibody development’, Clin Cancer Res, 14: 7624-34.
Sahin, U., M. Schuler, H. Richly, S. Bauer, A. Krilova, T. Dechow, M. Jerling, M. Utsch, C. Rohde, K. Dhaene, C. Huber, and O. Tureci. 2018. ‘A phase I dose-escalation study of IMAB362 (Zolbetuximab) in patients with advanced gastric and gastro-oesophageal junction cancer’, Eur J Cancer, 100: 17-26.
Saldanha, J. W. 2014. ‘Humanization Strategies.’ in S. Duebel and J. M. Reichert (eds.), Handbook of Therapeutic Antibodies, Second Edition (Wiley-VCH Verlag GmbH & Co. KGaA).
Shang, L., B. Daubeuf, M. Triantafilou, R. Olden, F. Depis, A. C. Raby, S. Herren, A. Dos Santos, P. Malinge, I. Dunn-Siegrist, S. Benmkaddem, A. Geinoz, G. Magistrelli, F. Rousseau, V. Buatois, S. Salgado-Pires, W. Reith, R. Monteiro, J. Pugin, O. Leger, W. Ferlin, M. Kosco-Vilbois, K. Triantafilou, and G. Elson. 2014. ‘Selective antibody intervention of Toll-like receptor 4 activation through Fc gamma receptor tethering’, J Biol Chem, 289: 15309-18.
Shields, R. L., A. K. Namenuk, K. Hong, Y. G. Meng, J. Rae, J. Briggs, D. Xie, J. Lai, A. Stadlen, B. Li, J. A. Fox, and L. G. Presta. 2001. ‘High resolution mapping of the binding site on human IgG1 for Fc gamma RI, Fc gamma MI, Fc gamma MIT, and FcRn and design of IgG1 variants with improved binding to the Fc gamma R’, J Biol Chem, 276: 6591-604.
Stavenhagen, J. B., S. Gorlatov, N. Tuaillon, C. T. Rankin, H. Li, S. Burke, L. Huang, S. Vijh, S. Johnson, E. Bonvini, and S. Koenig. 2007. ‘Fc optimization of therapeutic antibodies enhances their ability to kill tumor cells in vitro and controls tumor expansion in vivo via low-affinity activating Fcgamma receptors’, Cancer Res, 67: 8882-90.
Tao, M. H., and S. L. Morrison. 1989. ‘Studies of aglycosylated chimeric mouse-human IgG. Role of carbohydrate in the structure and effector functions mediated by the human IgG constant region’, J Immunol, 143: 2595-601.
Tureci, O., U. Sahin, H. Schulze-Bergkamen, Z. Zvirbule, F. Lordick, D. Koeberle, P. Thuss-Patience, T. Ettrich, D. Arnold, F. Bassermann, S. E. Al-Batran, K. Wiechen, K. Dhaene, D. Maurus, M. Gold, C. Huber, A. Krivoshik, A. Arozullah, J. W. Park, and M. Schuler. 2019. ‘A multicentre, phase 2a study of zolbetuximab as a single agent in patients with recurrent or refractory advanced adenocarcinoma of the stomach or lower oesophagus: the MONO study’, Ann Oncol.
Vafa, O., G. L. Gilliland, R. J. Brerski, B. Strake, T. Wilkinson, E. R. Lacy, B. Scallon, A. Teplyakov, T. J. Malia, and W. R. Strohl. 2014. ‘An engineered Fc variant of an IgG eliminates all immune effector functions via structural perturbations’, Methods, 65: 114-26.
Waldmeier, L., I. Hellmann, C. K. Gutknecht, F. I. Wolter, S. C. Cook, S. T. Reddy, U. Grawunder, and R. R. Beerli. 2016. ‘Transpo-mAb display: Transposition-mediated B cell display and functional screening of full-length IgG antibody libraries’, MAbs, 8: 726-40.
Walker, M. R., J. Lund, K. M. Thompson, and R. Jefferis. 1989. ‘Aglycosylation of human IgG1 and IgG3 monoclonal antibodies can eliminate recognition by human cells expressing Fc gamma RI and/or Fc gamma RII receptors’, Biochem J, 259: 347-53.
Wang, X., M. Mathieu, and R. J. Brerski. 2018. ‘IgG Fc engineering to modulate antibody effector functions’, Protein Cell, 9: 63-73.
Xu, D., M. L. Alegre, S. S. Varga, A. L. Rothermel, A. M. Collins, V. L. Pulito, L. S. Hanna, K. P. Dolan, P. W. Parren, J. A. Bluestone, L. K. Jolliffe, and R. A. Zivin. 2000. ‘In vitro characterization of five humanized OKT3 effector function variant antibodies’, Cell Immunol, 200: 16-26.
Yu, D., and J. R. Turner. 2008. ‘Stimulus-induced reorganization of tight junction structure: the role of membrane traffic’, Biochim Biophys Acta, 1778: 709-16.
Zalevsky, J., A. K. Chamberlain, H. M. Horton, S. Karki, I. W. Leung, T. J. Sproule, G. A. Lazar, D. C. Roopenian, and J. R. Desjarlais. 2010. ‘Enhanced antibody half-life improves in vivo activity’, Nat Biotechnol, 28: 157-9.
CN109762067
WO2000/015659
WO2004/047863
WO2005/113587
WO2007/059997
WO2008/145338
WO2013/167259
WO2013/174509
WO2014/075788
WO2014/127906
WO2016/166122
WO2016/165762
WO2018/006882
WO2019/173420
WO2019/175617
WO2019/219089

1. An antibody or fragment thereof binding to CLDN18.2, wherein the antibody or fragment thereof exhibits increased binding to tumor tissue expressing CLDN18.2 over healthy tissue expressing CLDN18.2.

2. An antibody or fragment thereof binding to CLDN18.2 comprising the HCDR1, HCDR2 and HCDR3 sequences of SEQ ID NO: 21, SEQ ID NO: 22, and SEQ ID NO: 23, respectively, and the LCDR1, LCDR2 and LCDR3 sequences of SEQ ID NO: 24, SEQ ID NO: 25, and SEQ ID NO: 26, respectively.

3. The antibody or fragment thereof of claim 1 or 2, comprising:
a. the HCDR1, HCDR2 and HCDR3 sequences of SEQ ID NO: 1, SEQ ID NO: 15 and SEQ ID NO: 3, respectively, and the LCDR1, LCDR2 and LCDR3 sequences of SEQ ID NO: 4, SEQ ID NO: 5 and SEQ ID NO: 6, respectively;
b. the HCDR1, HCDR2 and HCDR3 sequences of SEQ ID NO: 1, SEQ ID NO: 16 and SEQ ID NO: 3, respectively, and the LCDR1, LCDR2 and LCDR3 sequences of SEQ ID NO: 4, SEQ ID NO: 5 and SEQ ID NO: 6, respectively;
c. the HCDR1, HCDR2 and HCDR3 sequences of SEQ ID NO: 1, SEQ ID NO: 16 and SEQ ID NO: 3, respectively, and the LCDR1, LCDR2 and LCDR3 sequences of SEQ ID NO: 17, SEQ ID NO: 14 and SEQ ID NO: 11, respectively;
d. the HCDR1, HCDR2 and HCDR3 sequences of SEQ ID NO: 1, SEQ ID NO: 16 and SEQ ID NO: 3, respectively, and the LCDR1, LCDR2 and LCDR3 sequences of SEQ ID NO: 18, SEQ ID NO: 19 and SEQ ID NO: 11, respectively;
e. the HCDR1, HCDR2 and HCDR3 sequences of SEQ ID NO: 12, SEQ ID NO: 15 and SEQ ID NO: 3, respectively, and the LCDR1, LCDR2 and LCDR3 sequences of SEQ ID NO: 4, SEQ ID NO: 5 and SEQ ID NO: 6, respectively;
f. the HCDR1, HCDR2 and HCDR3 sequences of SEQ ID NO: 1, SEQ ID NO: 20 and SEQ ID NO: 3, respectively, and the LCDR1, LCDR2 and LCDR3 sequences of SEQ ID NO: 4, SEQ ID NO: 5 and SEQ ID NO: 6, respectively;
g. the HCDR1, HCDR2 and HCDR3 sequences of SEQ ID NO: 1, SEQ ID NO: 20 and SEQ ID NO: 3, respectively, and the LCDR1, LCDR2 and LCDR3 sequences of SEQ ID NO: 18, SEQ ID NO: 19 and SEQ ID NO: 11, respectively;
h. the HCDR1, HCDR2 and HCDR3 sequences of SEQ ID NO: 12, SEQ ID NO: 20 and SEQ ID NO: 8, respectively, and the LCDR1, LCDR2 and LCDR3 sequences of SEQ ID NO: 4, SEQ ID NO: 5 and SEQ ID NO: 6, respectively; or
i. the HCDR1, HCDR2 and HCDR3 sequences of SEQ ID NO: 12, SEQ ID NO: 20 and SEQ ID NO: 8, respectively, and the LCDR1, LCDR2 and LCDR3 sequences of SEQ ID NO: 17, SEQ ID NO: 14 and SEQ ID NO: 11, respectively.

4. The antibody or fragment thereof of claim 1 or 2, comprising:
a. the HCDR1, HCDR2 and HCDR3 sequences of SEQ ID NO: 1, SEQ ID NO: 2 and SEQ ID NO: 3, respectively, and the LCDR1, LCDR2 and LCDR3 sequences of SEQ ID NO: 4, SEQ ID NO: 5 and SEQ ID NO: 6, respectively;
b. the HCDR1, HCDR2 and HCDR3 sequences of SEQ ID NO: 1, SEQ ID NO: 7 and SEQ ID NO: 8, respectively, and the LCDR1, LCDR2 and LCDR3 sequences of SEQ ID NO: 9, SEQ ID NO: 10 and SEQ ID NO: 11, respectively; or
c. the HCDR1, HCDR2 and HCDR3 sequences of SEQ ID NO: 12, SEQ ID NO: 2 and SEQ ID NO: 3, respectively, and the LCDR1, LCDR2 and LCDR3 sequences of SEQ ID NO: 13, SEQ ID NO: 14 and SEQ ID NO: 11, respectively,
preferably comprising:
d. a VH sequence of SEQ ID NO: 27 and a VL sequence of SEQ ID NO: 28;
e. a VH sequence of SEQ ID NO: 29 and a VL sequence of SEQ ID NO: 30; or
f. a VH sequence of SEQ ID NO: 31 and a VL sequence of SEQ ID NO: 32.

5. The antibody or fragment thereof of any one of claims 1-3, comprising:
a. a VH sequence having at least 80%, at least 85%, at least 90%, at least 95% or at least 98% sequence identity with the amino acid sequence of SEQ ID NO: 33;
b. a VH sequence having at least 80%, at least 85%, at least 90%, at least 95% or at least 98% sequence identity with the amino acid sequence of SEQ ID NO: 34;
c. a VH sequence having at least 80%, at least 85%, at least 90%, at least 95% or at least 98% sequence identity with the amino acid sequence of SEQ ID NO: 35;
d. a VH sequence having at least 80%, at least 85%, at least 90%, at least 95% or at least 98% sequence identity with the amino acid sequence of SEQ ID NO: 36; or
e. a VH sequence having at least 80%, at least 85%, at least 90%, at least 95% or at least 98% sequence identity with the amino acid sequence of SEQ ID NO: 37; and
f. a VL sequence having at least 80%, at least 85%, at least 90%, at least 95% or at least 98% sequence identity with the amino acid sequence of SEQ ID NO: 38;
g. a VL sequence having at least 80%, at least 85%, at least 90%, at least 95% or at least 98% sequence identity with the amino acid sequence of SEQ ID NO: 39;
h. a VL sequence having at least 80%, at least 85%, at least 90%, at least 95% or at least 98% sequence identity with the amino acid sequence of SEQ ID NO: 40; or
i. a VL sequence having at least 80%, at least 85%, at least 90%, at least 95% or at least 98% sequence identity with the amino acid sequence of SEQ ID NO: 41,
preferably comprising:
a. a VH sequence of: SEQ ID NO: 33;
b. a VH sequence of SEQ ID NO: 34;
c. a VH sequence of SEQ ID NO: 35;
d. a VH sequence of SEQ ID NO: 36; or
e. a VH sequence of SEQ ID NO: 37;
and
f. a VL sequence of SEQ ID NO: 38;
g. a VL sequence of SEQ ID NO: 39;
h. a VL sequence of SEQ ID NO: 40; or
i. a VL sequence of SEQ ID NO: 41.

6. The antibody or fragment thereof of claim 1 or 2, comprising:
a. a VH sequence of SEQ ID NO: 33 and a VL sequence of SEQ ID NO: 38;
b. a VH sequence of SEQ ID NO: 34 and a VL sequence of SEQ ID NO: 38;
c. a VH sequence of SEQ ID NO: 34 and a VL sequence of SEQ ID NO: 39;
d. a VH sequence of SEQ ID NO: 34 and a VL sequence of SEQ ID NO: 40;
e. a VH sequence of SEQ ID NO: 35 and a VL sequence of SEQ ID NO: 38;
f. a VH sequence of SEQ ID NO: 36 and a VL sequence of SEQ ID NO: 41;
g. a VH sequence of SEQ ID NO: 36 and a VL sequence of SEQ ID NO: 40;
h. a VH sequence of SEQ ID NO: 37 and a VL sequence of SEQ ID NO: 41;
i. a VH sequence of SEQ ID NO: 37 and a VL sequence of SEQ ID NO: 38; or
j. a VH sequence of SEQ ID NO: 37 and a VL sequence of SEQ ID NO: 39.

7. The antibody of any one of claims 1-3, comprising:
a. a heavy chain sequence having at least 80%, at least 85%, at least 90%, at least 95% or at least 98% sequence identity with the amino acid sequence of the heavy chain sequence of SEQ ID NO: 46 and a light chain sequence having at least 80%, at least 85%, at least 90%, at least 95% or at least 98% sequence identity with the amino acid sequence of the light chain sequence of SEQ ID NO: 51;
b. a heavy chain sequence having at least 80%, at least 85%, at least 90%, at least 95% or at least 98% sequence identity with the amino acid sequence of the heavy chain sequence of SEQ ID NO: 47 and a light chain sequence having at least 80%, at least 85%, at least 90%, at least 95% or at least 98% sequence identity with the amino acid sequence of the light chain sequence of SEQ ID NO: 51;
c. a heavy chain sequence having at least 80%, at least 85%, at least 90%, at least 95% or at least 98% sequence identity with the amino acid sequence of the heavy chain sequence of SEQ ID NO: 47 and a light chain sequence having at least 80%, at least 85%, at least 90%, at least 95% or at least 98% sequence identity with the amino acid sequence of the light chain sequence of SEQ ID NO: 52;
d. a heavy chain sequence having at least 80%, at least 85%, at least 90%, at least 95% or at least 98% sequence identity with the amino acid sequence of the heavy chain sequence of SEQ ID NO: 47 and a light chain sequence having at least 80%, at least 85%, at least 90%, at least 95% or at least 98% sequence identity with the amino acid sequence of the light chain sequence of SEQ ID NO: 53;
e. a heavy chain sequence having at least 80%, at least 85%, at least 90%, at least 95% or at least 98% sequence identity with the amino acid sequence of the heavy chain sequence of SEQ ID NO: 48 and a light chain sequence having at least 80%, at least 85%, at least 90%, at least 95% or at least 98% sequence identity with the amino acid sequence of the light chain sequence of SEQ ID NO: 51;
f. a heavy chain sequence having at least 80%, at least 85%, at least 90%, at least 95% or at least 98% sequence identity with the amino acid sequence of the heavy chain sequence of SEQ ID NO: 47 and a light chain sequence having at least 80%, at least 85%, at least 90%, at least 95% or at least 98% sequence identity with the amino acid sequence of the light chain sequence of SEQ ID NO: 54;
g. a heavy chain sequence having at least 80%, at least 85%, at least 90%, at least 95% or at least 98% sequence identity with the amino acid sequence of the heavy chain sequence of SEQ ID NO: 49 and a light chain sequence having at least 80%, at least 85%, at least 90%, at least 95% or at least 98% sequence identity with the amino acid sequence of the light chain sequence of SEQ ID NO: 53;
h. a heavy chain sequence having at least 80%, at least 85%, at least 90%, at least 95% or at least 98% sequence identity with the amino acid sequence of the heavy chain sequence of SEQ ID NO: 50 and a light chain sequence having at least 80%, at least 85%, at least 90%, at least 95% or at least 98% sequence identity with the amino acid sequence of the light chain sequence of SEQ ID NO: 54;
i. a heavy chain sequence having at least 80%, at least 85%, at least 90%, at least 95% or at least 98% sequence identity with the amino acid sequence of the heavy chain sequence of SEQ ID NO: 50 and a light chain sequence having at least 80%, at least 85%, at least 90%, at least 95% or at least 98% sequence identity with the amino acid sequence of the light chain sequence of SEQ ID NO: 51;
j. a heavy chain sequence having at least 80%, at least 85%, at least 90%, at least 95% or at least 98% sequence identity with the amino acid sequence of the heavy chain sequence of SEQ ID NO: 50 and a light chain sequence having at least 80%, at least 85%, at least 90%, at least 95% or at least 98% sequence identity with the amino acid sequence of the light chain sequence of SEQ ID NO: 52
preferably comprising:
k. the heavy chain sequence of SEQ ID NO: 46 and light chain sequence of SEQ ID NO: 51;
l. the heavy chain sequence of SEQ ID NO: 47 and light chain sequence of SEQ ID NO: 51;
m. the heavy chain sequence of SEQ ID NO: 47 and light chain sequence of SEQ ID NO: 52;
n. the heavy chain sequence of SEQ ID NO: 47 and light chain sequence of SEQ ID NO: 53;
o. the heavy chain sequence of SEQ ID NO: 48 and light chain sequence of SEQ ID NO: 51;
p. the heavy chain sequence of SEQ ID NO: 47 and light chain sequence of SEQ ID NO: 54;
q. the heavy chain sequence of SEQ ID NO: 49 and light chain sequence of SEQ ID NO: 53;
r. the heavy chain sequence of SEQ ID NO: 50 and light chain sequence of SEQ ID NO: 54;
s. the heavy chain sequence of SEQ ID NO: 50 and light chain sequence of SEQ ID NO: 51;
t. the heavy chain sequence of SEQ ID NO: 50 and light chain sequence of SEQ ID NO: 52,
or versions thereof with an engineered Fc domain.

8. An antibody or fragment thereof binding to CLDN18.2, wherein the antibody or fragment thereof
(i) binds to the same epitope as an antibody comprising a heavy chain sequence of SEQ ID NO: 46 and a light chain sequence of SEQ ID NO: 51;
(ii) competes for binding with an antibody comprising a heavy chain sequence of SEQ ID NO: 46 and a light chain sequence of SEQ ID NO: 51; and/or
(iii) competitively inhibits binding of an antibody comprising a heavy chain sequence of SEQ ID NO: 46 and a light chain sequence of SEQ ID NO: 51 to CLDN18.2.

9. The antibody or fragment thereof of any one of claims 1 to 8, wherein the antibody or fragment thereof
a. is IgA1, IgA2, IgD, IgE, IgG1, IgG2, IgG3, IgG4, synthetic IgG, IgM, F(ab)₂, Fv, scFv, IgGACH2, F(ab′) 2, scFvCH3, Fab, VL, VH, scFv4, scFv3, scFv2, dsFv, Fv, scFv-Fc, (scFv)₂, a non-depleting IgG, a diabody, a bivalent antibody or Fc-engineered versions thereof;
b. is humanized;
c. does not bind to CLDN18.1;
d. is isolated; and/or
e. is less susceptible to posttranslational deamidation than IMAB362.

10. The antibody or fragment thereof of any one of claims 1 to 9, wherein the increased binding to tumor tissue expressing CLDN18.2 over healthy tissue expressing CLDN18.2 is measured by flow cytometry or by immunohistochemistry.

11. The antibody or fragment thereof of any one of claims 1 to 9 wherein the antibody or fragment thereof binds to CLDN18.2 expressed in HEK293T cells or PA-TU-8988-High cells with an EC50 value that is at least 1.1 times higher, at least 1.2 times higher, at least 1.5 times higher, at least 2 times higher or at least 2.5 times higher but not more than 3 times higher than the EC50 value of IMAB362 binding to CLDN18.2 expressed in HEK293T cells or PA-TU-8988-High cells, optionally wherein binding is measured by flow cytometry (FC) titration.

12. A nucleic acid encoding the antibody or fragment thereof of any of claims 1 to 11.

13. A vector comprising the nucleic acid of claim 12.

14. A host cell comprising the nucleic acid of claim 12 or the vector of claim 13.

15. The antibody or fragment thereof of any one of claims 1 to 11, the nucleic acid of claim 12, the vector of claim 13 or the host cell of claim 14 for use in the treatment of a subject
a. suffering from,
b. at risk of developing, and/or
c. being diagnosed for
a neoplastic disease; optionally wherein the neoplastic disease is selected from the group consisting of pancreatic, gastric, esophageal, ovarian and lung cancer.


SEQUENCE LISTING (133 sequences)

SEQ ID NO: 1 - protein, 5 aa, Artificial Sequence
  cCl1-1, cCl1-2, hCl1a, hCl1b, hCl1c, hCl1d, hCl1f, hCl1g HCDR1
DYAMH

SEQ ID NO: 2 - protein, 17 aa, Artificial Sequence
  cCl1-1, cCl1-3 HCDR2
WINTYTGKPTYADDFKG

SEQ ID NO: 3 - protein, 10 aa, Artificial Sequence
  cCl1-1, cCl1-3, hCl1a, hCl1b, hCl1c, hCl1d, hCl1e, hCl1f, hCl1g HCDR3
AVFYGYTMDA

SEQ ID NO: 4 - protein, 11 aa, Artificial Sequence
  cCl1-1, hCl1a, hCl1b, hCl1e, hCl1f, hCl1h, hCl1i LCDR1
RASEDIYSNLA

SEQ ID NO: 5 - protein, 7 aa, Artificial Sequence
  cCl1-1, hCl1a, hCl1b, hCl1e, hCl1f, hCl1h, hCl1i LCDR2
SVKRLQD

SEQ ID NO: 6 - protein, 9 aa, Artificial Sequence
  cCl1-1, hCl1a, hCl1b, hCl1e, hCl1f, hCl1h, hCl1i LCDR3
LQGSNFPLT

SEQ ID NO: 7 - protein, 17 aa, Artificial Sequence
  cCl1-2 HCDR2
WINAYTGKPTYADDFKG

SEQ ID NO: 8 - protein, 10 aa, Artificial Sequence
  cCl1-2, hCl1h, hCl1i, hCl1j HCDR3
AVYYGYTMDA

SEQ ID NO: 9 - protein, 11 aa, Artificial Sequence
  cCl1-2 LCDR1
RTSEDIYSNFA

SEQ ID NO: 10 - protein, 7 aa, Artificial Sequence
  cCl1-2 LCDR2
SVNRLQD

SEQ ID NO: 11 - protein, 9 aa, Artificial Sequence
  cCl1-2, cCl1-3, hCl1c, hCl1d, hCl1g, hCl1j LCDR3
LQGSKFPLT

SEQ ID NO: 12 - protein, 5 aa, Artificial Sequence
  cCl1-3, hCl1e, hCl1h, hCl1i, hCl1j HCDR1
DYAMY

SEQ ID NO: 13 - protein, 11 aa, Artificial Sequence
  cCl1-3 LCDR1
RTSEDIYSNLA

SEQ ID NO: 14 - protein, 7 aa, Artificial Sequence
  cCl1-3, hCl1c, hCl1j LCDR2
AIKRLQD

SEQ ID NO: 15 - protein, 17 aa, Artificial Sequence
  hCl1a, hCl1e HCDR2
WINTYTGKPTYAQKFQG

SEQ ID NO: 16 - protein, 17 aa, Artificial Sequence
  hCl1b, hCl1c, hCl1d HCDR2
WINTYTGKPTYSQKFQG

SEQ ID NO: 17 - protein, 11 aa, Artificial Sequence
  hCl1c, hCl1j LCDR1
RTSEDIYSNLA

SEQ ID NO: 18 - protein, 11 aa, Artificial Sequence
  hCl1d, hCl1g LCDR1
RTSEDIYSNFA

SEQ ID NO: 19 - protein, 7 aa, Artificial Sequence
  hCl1d, hCl1g LCDR2
SVNRLQD

SEQ ID NO: 20 - protein, 17 aa, Artificial Sequence
  hCl1f, hCl1g, hCl1h, hCl1i, hCl1j HCDR2
WINAYTGKPTYAQKFQG

SEQ ID NO: 21 - protein, 5 aa, Artificial Sequence
  HCDR1 consensus sequence
  MISC_FEATURE (5)..(5): H or Y
DYAMX

SEQ ID NO: 22 - protein, 17 aa, Artificial Sequence
  HCDR2 consensus sequence
  MISC_FEATURE (4)..(4): T or A
  MISC_FEATURE (12)..(12): A or S
  MISC_FEATURE (13)..(13): D or Q
  MISC_FEATURE (14)..(14): D or K
  MISC_FEATURE (16)..(16): K or Q
WINXYTGKPTYXXXFXG

SEQ ID NO: 23 - protein, 10 aa, Artificial Sequence
  HCDR3 consensus sequence
  MISC_FEATURE (3)..(3): F or Y
AVXYGYTMDA

SEQ ID NO: 24 - protein, 11 aa, Artificial Sequence
  LCDR1 consensus sequence
  MISC_FEATURE (2)..(2): A or T
  MISC_FEATURE (10)..(10): L or F
RXSEDIYSNXA

SEQ ID NO: 25 - protein, 7 aa, Artificial Sequence
  LCDR2 consensus sequence
  MISC_FEATURE (1)..(1): S or A
  MISC_FEATURE (2)..(2): V or I
  MISC_FEATURE (3)..(3): K or N
XXXRLQD

SEQ ID NO: 26 - protein, 9 aa, Artificial Sequence
  LCDR3 consensus sequence
  MISC_FEATURE (5)..(5): K or N
LQGSXFPLT

SEQ ID NO: 27 - protein, 119 aa, Artificial Sequence
  cCl1-1 HC variable region
QIQLVQSGPELKKPGESVKISCKASGYTFTDYAMHWVKQAPGKGLKWMGWINTYTGKPTYADDFKGRFVFSLEASASTANLQISNLKNEDTATYFCARAVFYGYTMDAWGQGTSVTVSS

SEQ ID NO: 28 - protein, 107 aa, Artificial Sequence
  cCl1-1 LC variable region
DIQMTQSPASLSASLGETISIACRASEDIYSNLAWYQQKSGKSPQLLIFSVKRLQDGVPSRFSGSGSGTQYSLKISGMQPEDEGDYFCLQGSNFPLTFGSGTKLEIK

SEQ ID NO: 29 - protein, 119 aa, Artificial Sequence
  cCl1-2 HC variable region
QIQLVQSGPELKKPGESVKISCKTSGYTFTDYAMHWVKQGPGKGMKWMGWINAYTGKPTYADDFKGRFVLSLEASASTANLQISNLKNEDTATYFCARAVYYGYTMDAWGQGTSVIVSS

SEQ ID NO: 30 - protein, 107 aa, Artificial Sequence
  cCl1-2 LC variable region
DIQMTQSPASLSASLGETISIECRTSEDIYSNFAWFQQKSGKSPQLLIYSVNRLQDGVPSRFSGSGSGTQYSLKISGMQPEDEGDYFCLQGSKFPLTFGSGTKLEIK

SEQ ID NO: 31 - protein, 119 aa, Artificial Sequence
  cCl1-3 HC variable region
QIQLVQSGPELKKPGESVKISCKASGYTFTDYAMYWVKQVPGKGLRWMGWINTYTGKPTYADDFKGRFVFSLEASASTANLQISNLKNEDTATYFCARAVFYGYTMDAWGQGTSVTVSS

SEQ ID NO: 32 - protein, 107 aa, Artificial Sequence
  cCl1-3 LC variable region
DIQMTQSPASLSASLGETISIACRTSEDIYSNLAWYQQKSGKSPQLLIFAIKRLQDGVPSRFSGSGSGTQYSLKISGMQPEDEGDYFCLQGSKFPLTFGSGTKLEIK

SEQ ID NO: 33 - protein, 119 aa, Artificial Sequence
  hCl1a HC variable region
QVQLVQSGAEVKKPGASVKVSCKASGYTFTDYAMHWVRQAPGQRLEWMGWINTYTGKPTYAQKFQGRVTITRDTSASTAYMELSSLRSEDTAVYYCARAVFYGYTMDAWGQGTLVTVSS

SEQ ID NO: 34 - protein, 119 aa, Artificial Sequence
  hCl1b, hCl1c and hCl1d HC variable region
QVQLVQSGAEVKKPGASVKVSCKASGYTFTDYAMHWVRQAPGQRLEWMGWINTYTGKPTYSQKFQGRVTITRDTSASTAYMELSSLRSEDTAVYYCARAVFYGYTMDAWGQGTLVTVSS

SEQ ID NO: 35 - protein, 119 aa, Artificial Sequence
  hCl1e HC variable region
QVQLVQSGAEVKKPGASVKVSCKASGYTFTDYAMYWVRQAPGQRLEWMGWINTYTGKPTYAQKFQGRVTITRDTSASTAYMELSSLRSEDTAVYYCARAVFYGYTMDAWGQGTLVTVSS

SEQ ID NO: 36 - protein, 119 aa, Artificial Sequence
  hCl1f and hCl1g HC variable region
QVQLVQSGAEVKKPGASVKVSCKASGYTFTDYAMHWVRQAPGQRLEWMGWINAYTGKPTYAQKFQGRVTITRDTSASTAYMELSSLRSEDTAVYYCARAVFYGYTMDAWGQGTLVTVSS

SEQ ID NO: 37 - protein, 119 aa, Artificial Sequence
  hCl1h, hCl1i and hCl1j HC variable region
QVQLVQSGAEVKKPGASVKVSCKASGYTFTDYAMYWVRQAPGQRLEWMGWINAYTGKPTYAQKFQGRVTITRDTSASTAYMELSSLRSEDTAVYYCARAVYYGYTMDAWGQGTLVTVSS

SEQ ID NO: 38 - protein, 107 aa, Artificial Sequence
  hCl1a, hCl1b, hCl1e and hCl1i LC variable region
DIQMTQSPSSLSASVGDRVTITCRASEDIYSNLAWYQQKPGKAPKLLIFSVKRLQDGVPSRFSGSGSGTDFTLTISSLQPEDFATYYCLQGSNFPLTFGQGTKVEIK

SEQ ID NO: 39 - protein, 107 aa, Artificial Sequence
  hCl1c and hCl1j LC variable region
DIQMTQSPSSLSASVGDRVTITCRTSEDIYSNLAWYQQKPGKAPKLLIFAIKRLQDGVPSRFSGSGSGTDFTLTISSLQPEDFATYYCLQGSKFPLTFGQGTKVEIK

SEQ ID NO: 40 - protein, 107 aa, Artificial Sequence
  hCl1d and hCl1g LC variable region
DIQMTQSPSSLSASVGDRVTITCRTSEDIYSNFAWYQQKPGKAPKLLIYSVNRLQDGVPSRFSGSGSGTDFTLTISSLQPEDFATYYCLQGSKFPLTFGQGTKVEIK

SEQ ID NO: 41 - protein, 107 aa, Artificial Sequence
  hCl1f and hCl1h LC variable region
DIQMTQSPSSLSASVGDRVTITCRASEDIYSNLAWYQQKPGKAPKLLIYSVKRLQDGVPSRFSGSGSGTDFTLTISSLQPEDFATYYCLQGSNFPLTFGQGTKVEIK

SEQ ID NO: 42 - protein, 119 aa, Artificial Sequence
  hCl3a, hCl1b and hCl1c HC variable region
QVQLQESGPGLVKPSETLSLTCAVSGYSVSSNYRWHWIRQPPGKGLEWIGYINIAGSTNYNPSLKSRVTISVDTSKNQFSLKLSSVTAADTAVYYCARNPSITRAMDAWGQGTLVTVSS

SEQ ID NO: 43 - protein, 107 aa, Artificial Sequence
  hCl3a LC variable region
DIQMTQSPSSLSASVGDRVTITCKSSQNIFKNLEWYQQKPGKAPKLLIYYTNNLQTGVPSRFSGSGSGTDFTLTISSLQPEDFATYYCYQYNSGPFTFGQGTKVEIK

SEQ ID NO: 44 - protein, 107 aa, Artificial Sequence
  hCl3b LC variable region
DIQMTQSPSSLSASVGDRVTITCRSSQNIFKNLEWYQQKPGKAPKLLIYYTNNLQTGVPSRFSGSGSGTDFTLTISSLQPEDFATYYCYQYNSGPFTFGQGTKVEIK

SEQ ID NO: 45 - protein, 107 aa, Artificial Sequence
  hCl3c LC variable region
DIQMTQSPSSLSASVGDRVTITCRSSQNIFKNLEWYQQKPGKAPKLLIYYTNNLQSGVPSRFSGSGSGTDFTLTISSLQPEDFATYYCYQYNSGPFTFGQGTKVEIK

SEQ ID NO: 46 - protein, 449 aa, Artificial Sequence
  hCl1a HC full
QVQLVQSGAEVKKPGASVKVSCKASGYTFTDYAMHWVRQAPGQRLEWMGWINTYTGKPTYAQKFQGRVTITRDTSASTAYMELSSLRSEDTAVYYCARAVFYGYTMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 449 aa, Artificial Sequence
  hCl1b, hCl1c and hCl1d HC full
QVQLVQSGAEVKKPGASVKVSCKASGYTFTDYAMHWVRQAPGQRLEWMGWINTYTGKPTYSQKFQGRVTITRDTSASTAYMELSSLRSEDTAVYYCARAVFYGYTMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 449 aa, Artificial Sequence
  hCl1e HC full
QVQLVQSGAEVKKPGASVKVSCKASGYTFTDYAMYWVRQAPGQRLEWMGWINTYTGKPTYAQKFQGRVTITRDTSASTAYMELSSLRSEDTAVYYCARAVFYGYTMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 449 aa, Artificial Sequence
  hCl1f and hCl1g HC full
QVQLVQSGAEVKKPGASVKVSCKASGYTFTDYAMHWVRQAPGQRLEWMGWINAYTGKPTYAQKFQGRVTITRDTSASTAYMELSSLRSEDTAVYYCARAVFYGYTMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 449 aa, Artificial Sequence
  hCl1h, hCl1i and hCl1j HC full
QVQLVQSGAEVKKPGASVKVSCKASGYTFTDYAMYWVRQAPGQRLEWMGWINAYTGKPTYAQKFQGRVTITRDTSASTAYMELSSLRSEDTAVYYCARAVYYGYTMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 214 aa, Artificial Sequence
  hCl1a, hCl1b, hCl1e and hCl1i LC full
DIQMTQSPSSLSASVGDRVTITCRASEDIYSNLAWYQQKPGKAPKLLIFSVKRLQDGVPSRFSGSGSGTDFTLTISSLQPEDFATYYCLQGSNFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 214 aa, Artificial Sequence
  hCl1c and hCl1j LC full
DIQMTQSPSSLSASVGDRVTITCRTSEDIYSNLAWYQQKPGKAPKLLIFAIKRLQDGVPSRFSGSGSGTDFTLTISSLQPEDFATYYCLQGSKFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 214 aa, Artificial Sequence
  hCl1d and hCl1g LC full
DIQMTQSPSSLSASVGDRVTITCRTSEDIYSNFAWYQQKPGKAPKLLIYSVNRLQDGVPSRFSGSGSGTDFTLTISSLQPEDFATYYCLQGSKFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 214 aa, Artificial Sequence
  hCl1f and hCl1h LC full
DIQMTQSPSSLSASVGDRVTITCRASEDIYSNLAWYQQKPGKAPKLLIYSVKRLQDGVPSRFSGSGSGTDFTLTISSLQPEDFATYYCLQGSNFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 448 aa, Artificial Sequence
  IMAB362 HC full
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 220 aa, Artificial Sequence
  IMAB362 LC full
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 10 aa, Artificial Sequence
  N-terminal extracellular of CLDN18.2, independent of glycosylation
DQWSTQDLYN

SEQ ID NO: 58 - protein, 11 aa, Artificial Sequence
  N-terminal extracellular of CLDN18.2, mainly unglycosylated
NNPVTAVFNYQ

SEQ ID NO: 59 - protein, 14 aa, Artificial Sequence
  N-terminal extracellular domain of CLDN18.2, unglycosylated
STQDLYNNPVTAVF

SEQ ID NO: 60 - protein, 13 aa, Artificial Sequence
  pan-CLDN18 peptide in the C-terminal extracellular domain common to both CLDN18.1 and CLDN18.2 isoforms
TNFWMSTANMYTG

SEQ ID NO: 61 - protein, 16 aa, Artificial Sequence
  Specific epitope of CLDN18.2 as disclosed by WO2005/113587
ALMIVGIVLGAIGLLV

SEQ ID NO: 62 - protein, 24 aa, Artificial Sequence
  Specific epitope of CLDN18.2 as disclosed by WO2005/113587
RIGSMEDSAKANMTLTSGIMFIVS

SEQ ID NO: 63 - protein, 129 aa, Artificial Sequence
  First extracellular domain of CLDN18.2 with N- and C-terminal extensions
METDTLLLWVLLLWVPGSTGDAAQPARRARRTKLGTELGSTPVWWNSADGRMDQWSTQDLYNNPVTAVFNYQGLWRSCVRESSGFTECRGYFTLLGLPAMLQAVRAAIQHSGGRSRRARTKTHLRRGSE

SEQ ID NO: 64 - protein, 15 aa, Artificial Sequence
  Overlapping peptide within the first extracellular domain as disclosed by WO2008/145338
MDQWSTQDLYNNPVT

SEQ ID NO: 65 - protein, 15 aa, Artificial Sequence
  Overlapping peptide within the first extracellular domain as disclosed by WO2008/145338
LYNNPVTAVFNYQGL

SEQ ID NO: 66 - protein, 15 aa, Artificial Sequence
  Overlapping peptide within the first extracellular domain as disclosed by WO2008/145338
VFNYQGLWRSCVRES

SEQ ID NO: 67 - protein, 15 aa, Artificial Sequence
  Overlapping peptide within the first extracellular domain as disclosed by WO2008/145338
QGLWRSCVRESSGFT

SEQ ID NO: 68 - protein, 15 aa, Artificial Sequence
  Overlapping peptide within the first extracellular domain as disclosed by WO2008/145338
RSCVRESSGFTECRG

SEQ ID NO: 69 - protein, 15 aa, Artificial Sequence
  C-terminal epitope of CLDN18.2 as disclosed by WO2013/167259
TEDEVQSYPSKHDYV

SEQ ID NO: 70 - protein, 12 aa, Artificial Sequence
  C-terminal epitope of CLDN18.2 as disclosed by WO2013/167259
EVQSYPSKHDYV

SEQ ID NO: 71 - DNA, 15 bp, Artificial Sequence
  cCl1-1, cCl1-2, hCl1a HCDR1
gactacgcgatgcac

SEQ ID NO: 72 - DNA, 51 bp, Artificial Sequence
  cCl1-1 HCDR2
tggatcaacacgtacacggggaagccgacatacgcggacgacttcaagggg

SEQ ID NO: 73 - DNA, 30 bp, Artificial Sequence
  cCl1-1, cCl1-3 HCDR3
gccgtcttctacggatatacgatggacgcg

SEQ ID NO: 74 - DNA, 357 bp, Artificial Sequence
  cCl1-1 VH
cagatccagctcgtccagagcgggccggagctgaagaagccgggggagagcgtgaagatctcgtgcaaggcgagcggatatacgttcacggactacgcgatgcactgggtcaagcaagcgccggggaaagggctgaagtggatggggtggatcaacacgtacacggggaagccgacatacgcggacgacttcaaggggcgattcgtgttctcgctggaggcgagcgcgagcacggcgaacctgcaaatctcgaacctgaagaacgaggacacggcgacgtacttctgcgcgcgggccgtcttctacggatatacgatggacgcgtgggggcagggtaccagcgtgacggtctcgagc

SEQ ID NO: 75 - DNA, 33 bp, Artificial Sequence
  cCl1-1 LCDR1
cgggcgagcgaggacatctactcgaacctggcg

SEQ ID NO: 76 - DNA, 21 bp, Artificial Sequence
  cCl1-1 LCDR2
tccgtcaagcggctgcaagac

SEQ ID NO: 77 - DNA, 27 bp, Artificial Sequence
  cCl1-1 LCDR3
ctgcaagggagcaacttcccgctgacg

SEQ ID NO: 78 - DNA, 321 bp, Artificial Sequence
  cCl1-1 VL
gacatccagatgacgcagagcccggcgtcgctgagcgcgagcctgggggagacgatctcgatcgcgtgccgggcgagcgaggacatctactcgaacctggcgtggtatcaacagaagagcgggaagagcccgcagctgctgatcttctccgtcaagcggctgcaagacggcgtcccgagccgattctcggggagcgggagcgggacgcagtactcgctgaagatctcggggatgcagccggaggacgagggggactacttctgcctgcaagggagcaacttcccgctgacgttcgggtcgggtaccaaactcgagatcaaa

SEQ ID NO: 79 - DNA, 51 bp, Artificial Sequence
  cCl1-2 HCDR2
tggatcaacgcgtacacggggaagccgacctacgcggacgacttcaagggg

SEQ ID NO: 80 - DNA, 27 bp, Artificial Sequence
  cCl1-2 HCDR3
gccgtctactacggatatacgatggac

SEQ ID NO: 81 - DNA, 357 bp, Artificial Sequence
  cCl1-2 VH
cagatccagctcgtccagagcgggccggagctgaagaagccgggggagagcgtgaagatctcgtgcaagacgagcggatatacgttcacggactacgcgatgcactgggtcaagcaggggccagggaaagggatgaagtggatggggtggatcaacgcgtacacggggaagccgacctacgcggacgacttcaaggggcgattcgtgctgagcctggaggcgagcgcctcgacggcgaacctgcaaatctcgaacctgaagaacgaggacacggcgacgtacttctgcgcgcgggccgtctactacggatatacgatggacgcgtgggggcagggtaccagcgtgatcgtctcgagc

SEQ ID NO: 82 - DNA, 33 bp, Artificial Sequence
  cCl1-2 LCDR1
cggacgagcgaggacatctactcgaacttcgcg

SEQ ID NO: 83 - DNA, 21 bp, Artificial Sequence
  cCl1-2 LCDR2
tcagtcaaccggctgcaagac

SEQ ID NO: 84 - DNA, 27 bp, Artificial Sequence
  cCl1-2, cCl1-3, hCl1d, hCl1g LCDR3
ctgcaagggagcaagttcccgctgacg

SEQ ID NO: 85 - DNA, 321 bp, Artificial Sequence
  cCl1-1 VL
gacatccagatgacgcagagcccggcgagcctgagcgcgagcctgggggagacgatctcgatcgagtgccggacgagcgaggacatctactcgaacttcgcgtggttccagcagaagagcgggaagagcccgcagctgctgatctactcagtcaaccggctgcaagacggcgtcccgagccgattctcggggagcgggagcgggacgcagtactcgctgaagatctcggggatgcagccggaggacgagggggactacttctgcctgcaagggagcaagttcccgctgacgttcgggagcggtaccaaactcgagatcaaa

SEQ ID NO: 86 - DNA, 15 bp, Artificial Sequence
  cCl1-3 HCDR1
gactacgcgatgtac

SEQ ID NO: 87 - DNA, 51 bp, Artificial Sequence
  cCl1-3 HCDR2
tggatcaacacgtacacggggaagccgacctacgcggacgacttcaagggg

SEQ ID NO: 88 - DNA, 357 bp, Artificial Sequence
  cCl1-3 VH
cagatccagctcgtccagagcgggccggagctgaagaagccgggggagagcgtgaagatctcgtgcaaggcgagcggatatacgttcacggactacgcgatgtactgggtcaagcaagtgccggggaaagggctgcgatggatggggtggatcaacacgtacacggggaagccgacctacgcggacgacttcaaggggcgattcgtgttctcgctggaggcgagcgcgagcacggcgaacctgcaaatctcgaacctgaagaacgaggacacggcgacgtacttctgcgcgcgggccgtcttctacggatatacgatggacgcgtgggggcagggtaccagcgtgacggtctcgagc

SEQ ID NO: 89 - DNA, 33 bp, Artificial Sequence
  cCl1-3 LCDR1
cggacgagcgaggacatctactcgaacctggcg

SEQ ID NO: 90 - DNA, 21 bp, Artificial Sequence
  cCl1-3 LCDR2
gcgatcaagcggctgcaagac

SEQ ID NO: 91 - DNA, 321 bp, Artificial Sequence
  cCl1-3 VL
gacatccagatgacgcagagcccggcgagcctgagcgcgagcctgggggagacgatctcgatcgcgtgccggacgagcgaggacatctactcgaacctggcgtggtatcaacagaagagcgggaagagcccgcagctgctgatcttcgcgatcaagcggctgcaagacggcgtcccgagccgattctcggggagcgggagcgggacgcagtactcgctgaagatctcggggatgcagccggaggacgagggggactacttctgcctgcaagggagcaagttcccgctgacgttcgggtcgggtaccaaactcgagatcaaa

SEQ ID NO: 92 - DNA, 51 bp, Artificial Sequence
  hCl1a HCDR2
tggatcaatacatacacggggaagccgacttatgcgcaaaaattccaagga

SEQ ID NO: 93 - DNA, 30 bp, Artificial Sequence
  hCl1a HCDR3
gcggtcttctacggatatacgatggatgcc

SEQ ID NO: 94 - DNA, 357 bp, Artificial Sequence
  cCl1a VH
caggtccaactagtccaaagcggggcggaagtcaagaagcccggagcatccgtcaaagtcagctgcaaggcgagcggatatacatttacggactacgcgatgcactgggtcaggcaagcccctgggcaaaggctcgaatggatgggatggatcaatacatacacggggaagccgacttatgcgcaaaaattccaaggaagagtcacaattacgcgggatacatccgcatctaccgcctacatggagctaagctcgctgcggagcgaggatacggcggtctactattgcgcccgagcggtcttctacggatatacgatggatgcctgggggcagggtaccctggtcacggtctcgagc

SEQ ID NO: 95 - DNA, 33 bp, Artificial Sequence
  hCl1a, hCl1b, hCl1e, hCl1i LCDR1
agggcctccgaagacatctactccaacctggca

SEQ ID NO: 96 - DNA, 21 bp, Artificial Sequence
  hCl1a, hCl1b, hCl1e, hCl1i LCDR2
agcgtcaaaagactacaagat

SEQ ID NO: 97 - DNA, 27 bp, Artificial Sequence
  hCl1a, hCl1b, hCl1e, hCl1i LCDR3
ttgcaaggaagcaatttccccttgact

SEQ ID NO: 98 - DNA, 321 bp, Artificial Sequence
  cCl1a, hCl1b, hCl1e, hCl1i VL
gacattcaaatgacgcaaagcccatcatcgctgagcgcatcggtcggggatagagtcaccataacatgcagggcctccgaagacatctactccaacctggcatggtatcaacaaaaaccggggaaggctccgaagctgctgatatttagcgtcaaaagactacaagatggagtaccgagccgattttcgggaagcgggagcgggacggatttcacgctgaccatatcaagtttgcaaccggaggattttgcgacatactattgcttgcaaggaagcaatttccccttgactttcgggcaaggtaccaaggtcgagatcaaa

SEQ ID NO: 99 - DNA, 15 bp, Artificial Sequence
  hCl1b, hCl1c, hCl1d HCDR1
gattatgcaatgcac

SEQ ID NO: 100 - DNA, 51 bp, Artificial Sequence
  hCl1b, hCl1c, hCl1d HCDR2
tggattaacacctacacgggcaagcccacatactcccaaaaattccaagga

SEQ ID NO: 101 - DNA, 30 bp, Artificial Sequence
  hCl1b, hCl1c, hCl1d HCDR3
gctgtattctatggatatacaatggatgcc

SEQ ID NO: 102 - DNA, 357 bp, Artificial Sequence
  hCl1b, hCl1c, hCl1d VH
caggtccaattagtccaaagcggggcggaagtcaagaagccgggggcgagcgtcaaagtctcatgcaaagcgagcggatacacatttacggattatgcaatgcactgggtcaggcaagcacccggacaaaggctggaatggatgggatggattaacacctacacgggcaagcccacatactcccaaaaattccaaggaagggtcacgataacgagagacacgagcgcgagcaccggaatggatgggatggattaacacctacacgggcaagcccacatactcccaaaaattccaaggaagggtcacgataacgagagacacgagcgcgagcaccgtaccctggtcaccgtctcgagc

SEQ ID NO: 103 - DNA, 33 bp, Artificial Sequence
  hCl1c, hCl1j LCDR1
cgaacgagcgaggacatatactcaaaccttgca

SEQ ID NO: 104 - DNA, 21 bp, Artificial Sequence
  hCl1c, hCl1j LCDR2
gcgataaagaggctgcaagac

SEQ ID NO: 105 - DNA, 27 bp, Artificial Sequence
  hCl1c, hCl1j LCDR3
ttgcaaggctccaaatttcccctgaca

SEQ ID NO: 106 - DNA, 321 bp, Artificial Sequence
  hCl1c, hCl1j VL
gacatccaaatgactcaaagcccatcatcgctatcggcatcggtcggggatagagtcacgataacatgccgaacgagcgaggacatatactcaaaccttgcatggtatcaacaaaagccggggaaggccccgaagctactgatattcgcgataaagaggctgcaagacggagttccatcacgattttcgggatctggctcggggaccgattttacgctgactatatcatcgctgcaaccggaagattttgcaacatactactgcttgcaaggctccaaatttcccctgacattcggacaaggtaccaaggtcgagatcaaa

SEQ ID NO: 107 - DNA, 33 bp, Artificial Sequence
  hCl1d, hCl1g LCDR1
cggacgagcgaggatatttattcgaactttgca

SEQ ID NO: 108 - DNA, 20 bp, Artificial Sequence
  hCl1d, hCl1g LCDR2
cagtcaatcggctacaagat

SEQ ID NO: 109 - DNA, 321 bp, Artificial Sequence
  hCl1d, hCl1g VL
gacatccaaatgacgcaatcaccgagctcgctgagcgcatctgtcggggaccgtgtcacaatcacatgccggacgagcgaggatatttattcgaactttgcatggtatcaacaaaaaccgggcaaggctccgaaacttttgatttattcagtcaatcggctacaagatggcgtcccgagccgatttagcgggagcggatcgggaaccgactttacgctgacgatatcatcgctacaaccggaggacttcgcgacttattactgcctacaagggagcaaattcccgctgacattcggacaaggtaccaaggtcgagatcaaa

SEQ ID NO: 110 - DNA, 15 bp, Artificial Sequence
  hCl1e HCDR1
gattacgcaatgtac

SEQ ID NO: 111 - DNA, 51 bp, Artificial Sequence
  hCl1e HCDR2
tggataaatacctatacgggaaagccaacatacgcccaaaaattccaaggc

SEQ ID NO: 112 - DNA, 30 bp, Artificial Sequence
  hCl1e HCDR3
gccgtcttttatggatatacgatggacgca

SEQ ID NO: 113 - DNA, 357 bp, Artificial Sequence
  hCl1e VH
caggtccaactggtccaatcgggggctgaagtcaaaaagccgggggcgagcgtcaaagtcagctgcaaagcatcgggatacacatttacggattacgcaatgtactgggtcaggcaagcacccggccaacgactggaatggatgggctggataaatacctatacgggaaagccaacatacgcccaaaaattccaaggccgcgtcacaataacgcgggacacgagcgcatcgacggcttatatggaactatcatcgctgcgatcggaagacacggcggtctattattgcgcacgcgccgtcttttatggatatacgatggacgcatgggggcagggtaccctggtcacggtctcgagc

SEQ ID NO: 114 - DNA, 15 bp, Artificial Sequence
  hCl1f, hCl1g HCDR1
gactacgcaatgcac

SEQ ID NO: 115 - DNA, 51 bp, Artificial Sequence
  hCl1f, hCl1g HCDR2
tggattaatgcctacacggggaagccgacctacgcacaaaaattccaagga

SEQ ID NO: 116 - DNA, 30 bp, Artificial Sequence
  hCl1f, hCl1g HCDR3
gccgtcttctatggatatacgatggatgct

SEQ ID NO: 117 - DNA, 357 bp, Artificial Sequence
  hCl1f, hCl1g VH
caggtccaattggtccaaagcggggcggaggtcaagaagccgggggcgagcgtcaaagtctcatgcaaggcaagcggatatacatttacggactacgcaatgcactgggtccggcaagcccctgggcaacggctggaatggatgggatggattaatgcctacacggggaagccgacctacgcacaaaaattccaaggacgagtcacgattacgcgggatactagcgcgagcaccgcatatatggagctaagctcgctgcgatctgaggataccgctgtatactactgcgcgagagccgtcttctatggatatacgatggatgcttgggggcagggtaccctggtcacggtctcgagc

SEQ ID NO: 118 - DNA, 33 bp, Artificial Sequence
  hCl1f, hCl1h LCDR1
cgagcttcggaggacatctatagcaacttggct

SEQ ID NO: 119 - DNA, 21 bp, Artificial Sequence
  hCl1f, hCl1h LCDR2
agcgtcaaaaggctccaagac

SEQ ID NO: 120 - DNA, 27 bp, Artificial Sequence
  hCl1f, hCl1h LCDR3
ctacaaggctctaacttcccattgaca

SEQ ID NO: 121 - DNA, 321 bp, Artificial Sequence
  hCl1f, hCl1h VL
gatatccaaatgacgcaatcaccatctagcctatcggcctctgtgggggaccgagtcaccatcacatgccgagcttcggaggacatctatagcaacttggcttggtatcaacaaaagccggggaaagcaccaaagctgctgatatatagcgtcaaaaggctccaagacggagtcccaagccgattctcgggctccggctccgggacggattttacgctgacaatttcgagcctgcaaccggaggactttgcaacctactattgcctacaaggctctaacttcccattgacatttgggcaaggtaccaaggtcgagatcaaa

SEQ ID NO: 122 - DNA, 15 bp, Artificial Sequence
  hCl1h, hCl1i, hCl1j HCDR1
gactacgctatgtat

SEQ ID NO: 123 - DNA, 51 bp, Artificial Sequence
  hCl1h, hCl1i, hCl1j HCDR2
tggattaatgcctacaccgggaagccgacttatgcgcaaaaatttcaagga

SEQ ID NO: 124 - DNA, 30 bp, Artificial Sequence
  hCl1h, hCl1i, hCl1j HCDR3
gcggtctactatggatatacgatggacgca

SEQ ID NO: 125 - DNA, 357 bp, Artificial Sequence
  hCl1h, hCl1i, hCl1j VH
caggtccaactggttcaatctggagcggaagtcaagaagcccggagcatccgtcaaagtctcgtgcaaggcatctggatacacattcaccgactacgctatgtattgggtccggcaagcccccggacaacggctggaatggatgggatggattaatgcctacaccgggaagccgacttatgcgcaaaaatttcaaggaagggtcacgattacgcgggacacgagcgcctcaaccgcatacatggagctatcgagcctgcgaagcgaggacaccgcggtctactactgcgcgcgggcggtctactatggatatacgatggacgcatgggggcagggtaccctggtcacggtctcgagc

SEQ ID NO: 126 - protein, 17 aa, Artificial Sequence
  HC CDR2 for hCl1x only, not chimeric clones cCl1-1,2,3
  MISC_FEATURE (4)..(4): T or A
  MISC_FEATURE (12)..(12): A or S
WINXYTGKPTYXQKFQG

SEQ ID NO: 127 - protein, 107 aa, Artificial Sequence [sequence omitted]

SEQ ID NO: 128 - protein, 330 aa, Artificial Sequence [sequence omitted]

SEQ ID NO: 129 - protein, 330 aa, Artificial Sequence
  L234A/L235A mutation in constant heavy chain - CH1 + Fc domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 330 aa, Artificial Sequence
  L236A/L236A/P329G mutation in constant heavy chain - CH1 + Fc domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 7 aa, Artificial Sequence
  Sortase tag
RLPQTGG

SEQ ID NO: 132 - protein, 11 aa, Artificial Sequence
  Sortase tag
GGGGSLPQTGG

SEQ ID NO: 133 - protein, 261 aa, Homo sapiens
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV